SEQUENCE LISTING (62 sequences)

SEQ ID NO: 1 - protein, 1944 aa, Homo sapiens
MSNFYEERTTMIAARDLQEFVPFGRDHCKHHPNALNLQLRQLQPASELWSSDGAAGLVGSLQEVTIHEKQKESWQLRKGVSEIGEDVDYDEELYVAGNMVIWSKGSKSQALAVYKAFTVDSPVQQALWCDFIISQDKSEKAYSSNEVEKCICILQSSCINMHSIEGKDYIASLPFQVANVWPTKYGLLFERSASSHEVPPGSPREPLPTMFSMLHPLDEITPLVCKSGSLFGSSRVQYVVDHAMKIVFLNTDPSIVMTYDAVQNVHSVWTLRRVKSEEENVVLKFSEQGGTPQNVATSSSLTAHLRSLSKGDSPVTSPFQNYSSIHSQSRSTSSPSLHSRSPSISNMAALSRAHSPALGVHSFSGVQRFNISSHNQSPKRHSISHSPNSNSNGSFLAPETEPIVPELCIDHLWTETITNIREKNSQASKVFITSDLCGQKFLCFLVESQLQLRCVKFQESNDKTQLIFGSVTNIPAKDAAPVEKIDTMLVLEGSGNLVLYTGVVRVGKVFIPGLPAPSLTMSNTMPRPSTPLDGVSTPKPLSKLLGSLDEVVLLSPVPELRDSSKLHDSLYNEDCTFQQLGTYIHSIRDPVHNRVTLELSNGSMVRITIPEIATSELVQTCLQAIKFILPKEIAVQMLVKWYNVHSAPGGPSYHSEWNLFVTCLMNMMGYNTDRLAWTRNFDFEGSLSPVIAPKKARPSETGSDDDWEYLLNSDYHQNVESHLLNRSLCLSPSEASQMKDEDFSQNLSLDSSTLLFTHIPAIFFVLHLVYEELKLNTLMGEGICSLVELLVQLARDLKLGPYVDHYYRDYPTLVRTTGQVCTIDPGQTGFMHHPSFFTSEPPSIYQWVSSCLKGEGMPPYPYLPGICERSRLVVLSIALYILGDESLVSDESSQYLTRITIAPQKLQVEQEENRFSFRHSTSVSSLAERLVVWMTNVGFTLRDLETLPFGIALPIRDAIYHCREQPASDWPEAVCLLIGRQDLSKQACEGNLPKGKSVLSSDVPSGTETEEEDDGMNDMNHEVMSLIWSEDLRVQDVRRLLQSAHPVRVNVVQYPELSDHEFIEEKENRLLQLCQRTMALPVGRGMFTLFSYHPVPTEPLPIPKLNLTGRAPPRNTTVDLNSGNIDVPPNMTSWASFHNGVAAGLKIAPASQIDSAWIVYNKPKHAELANEYAGFLMALGLNGHLTKLATLNIHDYLTKGHEMTSIGLLLGVSAAKLGTMDMSITRLLSIHIPALLPPTSTELDVPHNVQVAAVVGIGLVYQGTAHRHTAEVLLAEIGRPPGPEMEYCTDRESYSLAAGLALGMVCLGHGSNLIGMSDLNVPEQLYQYMVGGHRRFQTGMHREKHKSPSYQIKEGDTINVDVTCPGATLALAMIYLKTNNRSIADWLRAPDTMYLLDFVKPEFLLLRTLARCLILWDDILPNSKWVDSNVPQIIRENSISLSEIELPCSEDLNLETLSQAHVYIIAGACLSLGFRFAGSENLSAFNCLHKFAKDFMTYLSAPNASVTGPHNLETCLSVVLLSLAMVMAGSGNLKVLQLCRFLHMKTGGEMNYGFHLAHHMALGLLFLGGGRYSLSTSNSSIAALLCALYPHFPAHSTDNRYHLQALRHLYVLAAEPRLLVPVDVDTNTPCYALLEVTYKGTQWYEQTKEELMAPTLLPELHLLKQIKVKGPRYWELLIDLSKGTQHLKSILSKDGVLYVKLRAGQLSYKEDPMGWQSLLAQTVANRNSEARAFKPETISAFTSDPALLSFAEYFCKPTVNMGQKQEILDLFSSVLYECVTQETPEMLPAYIAMDQAIRRLGRREMSETSELWQIKLVLEFFSSRSHQERLQNHPKRGLFMNSEFLPVVKCTIDNTLDQWLQVGGDMCVHAYLSGQPLEESQLSMLACFLVYHSVPAPQHLPPIGLEGSTSFAELLFKFKQLKMPVRALLRLAPLLLGNPQPMVM

SEQ ID NO: 2 - protein, 185 aa, Homo sapiens
MTTPNKTPPGADPKQLERTGTVREIGSQAVWSLSSCKPGFGVDQLRDDNLETYWQSDGSQPHLVNIQFRRKTTVKTLCIYADYKSDESYTPSKISVRVGNNFHNLQEIRQLELVEPSGWIHVPLTDNHKKPTRTFMIQIAVLANHQNGRDTHMRQIKIYTPVEESSIGKFPRCTTIDFMMYRSIR

SEQ ID NO: 3 - protein, 84 aa, Homo sapiens
MKVKIKCWNGVATWLWVANDENCGICRMAFNGCCPDCKVPGDDCPLVWGQCSHCFHMHCILKWLHAQQVQQHCPMCRQEWKFKE

SEQ ID NO: 4 - protein, 74 aa, Homo sapiens
MDSEVQRDGRILDLIDDAWREDKLPYEDVAIPLNELPEPEQDNGGTTESVKEQEMKWTDLALQYLHENVPPIGN

SEQ ID NO: 5 - protein, 121 aa, Homo sapiens
MSTLFPSLFPRVTETLWFNLDRPCVEETELQQQEQQHQAWLQSIAEKDNNLVPIGKPASEHYDDEEEEDDEDDEDSEEDSEDDEDMQDMDEMNDYNESPDDGEVNEVDMEGNEQDQDQWMI

SEQ ID NO: 6 - protein, 110 aa, Homo sapiens
MAASSSSSSAGGVSGSSVTGSGFSVSDLAPPRKALFTYPKGAGEMLEDGSERFLCESVFSYQVASTLKQVKHDQQVARMEKLAGLVEELEADEWRFKPIEQLLGFTPSSG

SEQ ID NO: 7 - protein, 822 aa, Homo sapiens
MAAAVVVAEGDSDSRPGQELLVAWNTVSTGLVPPAALGLVSSRTSGAVPPKEEELRAAVEVLRGHGLHSVLEEWFVEVLQNDLQANISPEFWNAISQCENSADEPQCLLLLLDAFGLLESRLDPYLRSLELLEKWTRLGLLMGTGAQGLREEVHTMLRGVLFFSTPRTFQEMIQRLYGCFLRVYMQSKRKGEGGTDPELEGELDSRYARRRYYRLLQSPLCAGCSSDKQQCWCRQALEQFHQLSQVLHRLSLLERVSAEAVTTTLHQVTRERMEDRCRGEYERSFLREFHKWIERVVGWLGKVFLQDGPARPASPEAGNTLRRWRCHVQRFFYRIYASLRIEELFSIVRDFPDSRPAIEDLKYCLERTDQRQQLLVSLKAALETRLLHPGVNTCDIITLYISAIKALRVLDPSMVILEVACEPIRRYLRTREDTVRQIVAGLTGDSDGTGDLAVELSKTDPASLETGQDSEDDSGEPEDWVPDPVDADPGKSSSKRRSSDIISLLVSIYGSKDLFINEYRSLLADRLLHQFSFSPEREIRNVELLKLRFGEAPMHFCEVMLKDMADSRRINANIREEDEKRPAEEQPPFGVYAVILSSEFWPPFKDEKLEVPEDIRAALEAYCKKYEQLKAMRTLSWKHTLGLVTMDVELADRTLSVAVTPVQAVILLYFQDQASWTLEELSKAVKMPVALLRRRMSVWLQQGVLREEPPGTFSVIEEERPQDRDNMVLIDSDDESDSGMASQADQKEEELLLFWTYIQAMLTNLESLSLDRIYNMLRMFVVTGPALAEIDLQELQGYLQKKVRDQQLVYSAGVYRLPKNCS

SEQ ID NO: 8 - protein, 808 aa, Homo sapiens
MLRFPTCFPSFRVVGEKQLPQEIIFLVWSPKRDLIALANTAGEVLLHRLASFHRVWSFPPNENTGKEVTCLAWRPDGKLLAFALADTKKIVLCDVEKPESLHSFSVEAPVSCMHWMEVTVESSVLTSFYNAEDESNLLLPKLPTLPKNYSNTSKIFSEENSDEIIKLLGDVRLNILVLGGSSGFIELYAYGMFKIARVTGIAGTCLALCLSSDLKSLSVVTEVSTNGASEVSYFQLETNLLYSFLPEVTRMARKFTHISALLQYINLSLTCMCEAWEEILMQMDSRLTKFVQEKNTTTSVQDEFMHLLLWGKASAELQTLLMNQLTVKGLKKLGQSIESSYSSIQKLVISHLQSGSESLLYHLSELKGMASWKQKYEPLGLDAAGIEEAITAVGSFILKANELLQVIDSSMKNFKAFFRWLYVAMLRMTEDHVLPELNKMTQKDITFVAEFLTEHFNEAPDLYNRKGKYFNVERVGQYLKDEDDDLVSPPNTEGNQWYDFLQNSSHLKESPLLFPYYPRKSLHFVKRRMENIIDQCLQKPADVIGKSMNQAICIPLYRDTRSEDSTRRLFKFPFLWNNKTSNLHYLLFTILEDSLYKMCILRRHTDISQSVSNGLIAIKFGSFTYATTEKVRRSIYSCLDAQFYDDETVTVVLKDTVGREGRDRLLVQLPLSLVYNSEDSAEYQFTGTYSTRLDEQCSAIPTRTMHFEKHWRLLESMKAQYVAGNGFRKVSCVLSSNLRHVRVFEMDIDDEWELDESSDEEEEASNKPVKIKEEVLSESEAENQQAGAAALAPEIVIKVEKLDPELDS

SEQ ID NO: 9 - protein, 755 aa, Homo sapiens
MASVHESLYFNPMMTNGVVHANVFGIKDWVTPYKIAVLVLLNEMSRTGEGAVSLMERRRLNQLLLPLLQGPDITLSKLYKLIEESCPQLANSVQIRIKLMAEGELKDMEQFFDDLSDSFSGTEPEVHKTSVVGLFLRHMILAYSKLSFSQVFKLYTALQQYFQNGEKKTVEDADMELTSRDEGERKMEKEELDVSVREEEVSCSGPLSQKQAEFFLSQQASLLKNDETKALTPASLQKELNNLLKFNPDFAEAHYLSYLNNLRVQDVFSSTHSLLHYFDRLILTGAESKSNGEEGYGRSLRYAALNLAALHCRFGHYQQAELALQEAIRIAQESNDHVCLQHCLSWLYVLGQKRSDSYVLLEHSVKKAVHFGLPYLASLGIQSLVQQRAFAGKTANKLMDALKDSDLLHWKHSLSELIDISIAQKTAIWRLYGRSTMALQQAQMLLSMNSLEAVNAGVQQNNTESFAVALCHLAELHAEQGCFAAASEVLKHLKERFPPNSQHAQLWMLCDQKIQFDRAMNDGKYHLADSLVTGITALNSIEGVYRKAVVLQAQNQMSEAHKLLQKLLVHCQKLKNTEMVISVLLSVAELYWRSSSPTIALPMLLQALALSKEYRLQYLASETVLNLAFAQLILGIPEQALSLLHMAIEPILADGAILDKGRAMFLVAKCQVASAASYDQPKKAEALEAAIENLNEAKNYFAKVDCKERIRDVVYFQARLYHTLGKTQERNRCAMLFRQLHQELPSHGVPLINHL

SEQ ID NO: 10 - protein, 565 aa, Homo sapiens
MNVIDHVRDMAAAGLHSNVRLLSSLLLTMSNNNPELFSPPQKYQLLVYHADSLFHDKEYRNAVSKYTMALQQKKALSKTSKVRPSTGNSASTPQSQCLPSEIEVKYKMAECYTMLKQDKDAIAILDGIPSRQRTPKINMMLANLYKKAGQERPSVTSYKEVLRQCPLALDAILGLLSLSVKGAEVASMTMNVIQTVPNLDWLSVWIKAYAFVHTGDNSRAISTICSLEKKSLLRDNVDLLGSLADLYFRAGDNKNSVLKFEQAQMLDPYLIKGMDVYGYLLAREGRLEDVENLGCRLFNISDQHAEPWVVSGCHSFYSKRYSRALYLGAKAIQLNSNSVQALLLKGAALRNMGRVQEAIIHFREAIRLAPCRLDCYEGLIECYLASNSIREAMVMANNVYKTLGANAQTLTLLATVCLEDPVTQEKAKTLLDKALTQRPDYIKAVVKKAELLSREQKYEDGIALLRNALANQSDCVLHRILGDFLVAVNEYQEAMDQYSIALSLDPNDQKSLEGMQKMEKEESPTDATQEEDVDDMEGSGEEGDLEGSDSEAAQWADQEQWFGMQ

SEQ ID NO: 11 - protein, 620 aa, Homo sapiens
MNLERLRKRVRQYLDQQQYQSALFWADKVASLSREEPQDIYWLAQCLYLTAQYHRAAHALRSRKLDKLYEACRYLAARCHYAAKEHQQALDVLDMEEPINKRLFEKYLKDESGFKDPSSDWEMSQSSIKSSICLLRGKIYDALDNRTLATYSYKEALKLDVYCFEAFDLLTSHHMLTAQEEKELLESLPLSKLCNEEQELLRFLFENKLKKYNKPSETVIPESVDGLQENLDVVVSLAERHYYNCDFKMCYKLTSVVMEKDPFHASCLPVHIGTLVELNKANELFYLSHKLVDLYPSNPVSWFAVGCYYLMVGHKNEHARRYLSKATTLEKTYGPAWIAYGHSFAVESEHDQAMAAYFTAAQLMKGCHLPMLYIGLEYGLTNNSKLAERFFSQALSIAPEDPFVMHEVGVVAFQNGEWKTAEKWFLDALEKIKAIGNEVTVDKWEPLLNNLGHVCRKLKKYAEALDYHRQALVLIPQNASTYSAIGYIHSLMGNFENAVDYFHTALGLRRDDTFSVTMLGHCIEMYIGDSEAYIGADIKDKLKCYDFDVHTMKTLKNIISPPWDFREFEVEKQTAEETGLTPLETSRKTPDSRPSLEETFEIEMNESDMMLETSMSDHST

SEQ ID NO: 12 - protein, 597 aa, Homo sapiens
MAASTSMVPVAVTAAVAPVLSINSDFSDLREIKKQLLLIAGLTRERGLLHSSKWSAELAFSLPALPLAELQPPPPITEEDAQDMDAYTLAKAYFDVKEYDRAAHFLHGCNSKKAYFLYMYSRYLSGEKKKDDETVDSLGPLEKGQVKNEALRELRVELSKKHQARELDGFGLYLYGVVLRKLDLVKEAIDVFVEATHVLPLHWGAWLELCNLITDKEMLKFLSLPDTWMKEFFLAHIYTELQLIEEALQKYQNLIDVGFSKSSYIVSQIAVAYHNIRDIDKALSIFNELRKQDPYRIENMDTFSNLLYVRSMKSELSYLAHNLCEIDKYRVETCCVIGNYYSLRSQHEKAALYFQRALKLNPRYLGAWTLMGHEYMEMKNTSAAIQAYRHAIEVNKRDYRAWYGLGQTYEILKMPFYCLYYYRRAHQLRPNDSRMLVALGECYEKLNQLVEAKKCYWRAYAVGDVEKMALVKLAKLHEQLTESEQAAQCYIKYIQDIYSCGEIVEHLEESTAFRYLAQYYFKCKLWDEASTCAQKCCAFNDTREEGKALLRQILQLRNQGETPTTEVPAPFFLPASLSANNTPTRRVSPLNLSSVTP

SEQ ID NO: 13 - protein, 85 aa, Homo sapiens
MLRRKPTRLELKLDDIEEFENIRKDLETRKKQKEDVEVVGGSDGEGAIGLSSDPKSREQMINDRIGYKPQPKPNNRSSQFGSLEF

SEQ ID NO: 14 - protein, 824 aa, Homo sapiens
MTVLQEPVQAAIWQALNHYAYRDAVFLAERLYAEVHSEEALFLLATCYYRSGKAYKAYRLLKGHSCTTPQCKYLLAKCCVDLSKLAEGEQILSGGVFNKQKSHDDIVTEFGDSACFTLSLLGHVYCKTDRLAKGSECYQKSLSLNPFLWSPFESLCEIGEKPDPDQTFKFTSLQNFSNCLPNSCTTQVPNHSLSHRQPETVLTETPQDTIELNRLNLESSNSKYSLNTDSSVSYIDSAVISPDTVPLGTGTSILSKQVQNKPKTGRSLLGGPAALSPLTPSFGILPLETPSPGDGSYLQNYTNTPPVIDVPSTGAPSKKSVARIGQTGTKSVFSQSGNSREVTPILAQTQSSGPQTSTTPQVLSPTITSPPNALPRRSSRLFTSDSSTTKENSKKLKMKFPPKIPNRKTKSKTNKGGITQPNINDSLEITKLDSSIISEGKISTITPQIQAFNLQKAAAEGLMSLLREMGKGYLALCSYNCKEAINILSHLPSHHYNTGWVLCQIGRAYFELSEYMQAERIFSEVRRIENYRVEGMEIYSTTLWHLQKDVALSVLSKDLTDMDKNSPEAWCAAGNCFSLQREHDIAIKFFQRAIQVDPNYAYAYTLLGHEFVLTEELDKALACFRNAIRVNPRHYNAWYGLGMIYYKQEKFSLAEMHFQKALDINPQSSVLLCHIGVVQHALKKSEKALDTLNKAIVIDPKNPLCKFHRASVLFANEKYKSALQELEELKQIVPKESLVYFLIGKVYKKLGQTHLALMNFSWAMDLDPKGANNQIKEAIDKRYLPDDEEPITQEEQIMGTDESQESSMTDADDTQLHAAESDEF

SEQ ID NO: 15 - protein, 179 aa, Homo sapiens
MASQNRDPAATSVAAARKGAEPSGGAARGPVGKRLQQELMTLMMSGDKGISAFPESDNLFKWVGTIHGAAGTVYEDLRYKLSLEFPSGYPYNAPTVKFLTPCYHPNVDTQGNICLDILKEKWSALYDVRTILLSIQSLLGEPNIDSPLNTHAAELWKNPTAFKKYLQETYSKQVTSQEP

SEQ ID NO: 16 - protein, 147 aa, Homo sapiens
MALKRIQKELSDLQRDPPAHCSAGPVGDDLFHWQATIMGPPDSAYQGGVFFLTVHFPTDYPFKPPKIAFTTKIYHPNINSNGSICLDILRSQWSPALTVSKVLLSICSLLCDPNPDDPLVPDIAQIYKSDKEKYNRHAREWTQKYAM

SEQ ID NO: 17 - protein, 222 aa, Homo sapiens
MNSNVENLPPHIIRLVYKEVTTLTADPPDGIKVFPNEEDLTDLQVTIEGPEGTPYAGGLFRMKLLLGKDFPASPPKGYFLTKIFHPNVGANGEICVNVLKRDWTAELGIRHVLLTIKCLLIHPNPESALNEEAGRLLLENYEEYAARARLLTEIHGGAGGPSGRAEAGRALASGTEASSTDPGAPGGPGGAEGPMAKKHAGERDKKLAAKKKTDKKRALRRL

SEQ ID NO: 18 - protein, 1085 aa, Homo sapiens
MDTPENVLQMLEAHMQSYKGNDPLGEWERYIQWVEENFPENKEYLITLLEHLMKEFLDKKKYHNDPRFISYCLKFAEYNSDLHQFFEFLYNHGIGTLSSPLYIAWAGHLEAQGELQHASAVLQRGIQNQAEPREFLQQQYRLFQTRLTETHLPAQARTSEPLHNVQVLNQMITSKSNPGNNMACISKNQGSELSGVISSACDKESNMERRVITISKSEYSVHSSLASKVDVEQVVMYCKEKLIRGESEFSFEELRAQKYNQRRKHEQWVNEDRHYMKRKEANAFEEQLLKQKMDELHKKLHQVVETSHEDLPASQERSEVNPARMGPSVGSQQELRAPCLPVTYQQTPVNMEKNPREAPPVVPPLANAISAALVSPATSQSIAPPVPLKAQTVTDSMFAVASKDAGCVNKSTHEFKPQSGAEIKEGCETHKVANTSSFHTTPNTSLGMVQATPSKVQPSPTVHTKEALGFIMNMFQAPTLPDISDDKDEWQSLDQNEDAFEAQFQKNVRSSGAWGVNKIISSLSSAFHVFEDGNKENYGLPQPKNKPTGARTFGERSVSRLPSKPKEEVPHAEEFLDDSTVWGIRCNKTLAPSPKSPGDFTSAAQLASTPFHKLPVESVHILEDKENVVAKQCTQATLDSCEENMVVPSRDGKFSPIQEKSPKQALSSHMYSASLLRLSQPAAGGVLTCEAELGVEACRLTDTDAAIAEDPPDAIAGLQAEWMQMSSLGTVDAPNFIVGNPWDDKLIFKLLSGLSKPVSSYPNTFEWQCKLPAIKPKTEFQLGSKLVYVHHLLGEGAFAQVYEATQGDLNDAKNKQKFVLKVQKPANPWEFYIGTQLMERLKPSMQHMFMKFYSAHLFQNGSVLVGELYSYGTLLNAINLYKNTPEKVMPQGLVISFAMRMLYMIEQVHDCEIIHGDIKPDNFILGNGFLEQDDEDDLSAGLALIDLGQSIDMKLFPKGTIFTAKCETSGFQCVEMLSNKPWNYQIDYFGVAATVYCMLFGTYMKVKNEGGECKPEGLFRRLPHLDMWNEFFHVMLNIPDCHHLPSLDLLRQKLKKVFQQHYTNKIRALRNRLIVLLLECKRSRK

SEQ ID NO: 19 - protein, 1050 aa, Homo sapiens
MAAVKKEGGALSEAMSLEGDEWELSKENVQPLRQGRIMSTLQGALAQESACNNTLQQQKRAFEYEIRFYTGNDPLDVWDRYISWTEQNYPQGGKESNMSTLLERAVEALQGEKRYYSDPRFLNLWLKLGRLCNEPLDMYSYLHNQGIGVSLAQFYISWAEEYEARENFRKADAIFQEGIQQKAEPLERLQSQHRQFQARVSRQTLLALEKEEEEEVFESSVPQRSTLAELKSKGKKTARAPIIRVGGALKAPSQNRGLQNPFPQQMQNNSRITVFDENADEASTAELSKPTVQPWIAPPMPRAKENELQAGPWNTGRSLEHRPRGNTASLIAVPAVLPSFTPYVEETARQPVMTPCKIEPSINHILSTRKPGKEEGDPLQRVQSHQQASEEKKEKMMYCKEKIYAGVGEFSFEEIRAEVFRKKLKEQREAELLTSAEKRAEMQKQIEEMEKKLKEIQTTQQERTGDQQEETMPTKETTKLQIASESQKIPGMTLSSSVCQVNCCARETSLAENIWQEQPHSKGPSVPFSIFDEFLLSEKKNKSPPADPPRVLAQRRPLAVLKTSESITSNEDVSPDVCDEFTGIEPLSEDAIITGFRNVTICPNPEDTCDFARAARFVSTPFHEIMSLKDLPSDPERLLPEEDLDVKTSEDQQTACGTIYSQTLSIKKLSPIIEDSREATHSSGFSGSSASVASTSSIKCLQIPEKLELTNETSENPTQSPWCSQYRRQLLKSLPELSASAELCIEDRPMPKLEIEKEIELGNEDYCIKREYLICEDYKLFWVAPRNSAELTVIKVSSQPVPWDFYINLKLKERLNEDFDHFCSCYQYQDGCIVWHQYINCFTLQDLLQHSEYITHEITVLIIYNLLTIVEMLHKAEIVHGDLSPRCLILRNRIHDPYDCNKNNQALKIVDFSYSVDLRVQLDVFTLSGFRTVQILEGQKILANCSSPYQVDLFGIADLAHLLLFKEHLQVFWDGSFWKLSQNISELKDGELWNKFFVRILNANDEATVSVLGELAAEMNGVFDTTFQSHLNKALWKVGKLTSPGALLFQ

SEQ ID NO: 20 - protein, 328 aa, Homo sapiens
MTGSNEFKLNQPPEDGISSVKFSPNTSQFLLVSSWDTSVRLYDVPANSMRLKYQHTGAVLDCAFYDPTHAWSGGLDHQLKMHDLNTDQENLVGTHDAPIRCVEYCPEVNVMVTGSWDQTVKLWDPRTPCNAGTFSQPEKVYTLSVSGDRLIVGTAGRRVLVWDLRNMGYVQQRRESSLKYQTRCIRAFPNKQGYVLSSIEGRVAVEYLDPSPEVQKKKYAFKCHRLKENNIEQIYPVNAISFHNIHNTFATGGSDGFVNIWDPFNKKRLCQFHRYPTSIASLAFSNDGTTLAIASSYMYEMDDTEHPEDGIFIRQVTDAETKPKSPCT

SEQ ID NO: 21 - protein, 344 aa, Homo sapiens
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPMGAQDLISKLLRHNPSERLPLAQVSAHPWVRANSRRVLPPSALQSVA

SEQ ID NO: 22 - protein, 433 aa, Homo sapiens
MALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKV

SEQ ID NO: 23 - protein, 718 aa, Homo sapiens
MEDLGENTMVLSTLRSLNNFISQRVEGGSGLDISTSAPGSLQMQYQQSMQLEERAEQIRSKSHLIQVEREKMQMELSHKRARVELERAASTSARNYEREVDRNQELLTRIRQLQEREAGAEEKMQEQLERNRQCQQNLDAASKRLREKEDSLAQAGETINALKGRISELQWSVMDQEMRVKRLESEKQELQEQLDLQHKKCQEANQKIQELQASQEARADHEQQIKDLEQKLSLQEQDAAIVKNMKSELVRLPRLERELKQLREESAHLREMRETNGLLQEELEGLQRKLGRQEKMQETLVGLELENERLLAKLQSWERLDQTMGLSIRTPEDLSRFVVELQQRELALKDKNSAVTSSARGLEKARQQLQEELRQVSGQLLEERKKRETHEALARRLQKRVLLLTKERDGMRAILGSYDSELTPAEYSPQLTRRMREAEDMVQKVHSHSAEMEAQLSQALEELGGQKQRADMLEMELKMLKSQSSSAEQSFLFSREEADTLRLKVEELEGERSRLEEEKRMLEAQLERRALQGDYDQSRTKVLHMSLNPTSVARQRLREDHSQLQAECERLRGLLRAMERGGTVPADLEAAAASLPSSKEVAELKKQVESAELKNQRLKEVFQTKIQEFRKACYTLTGYQIDITTENQYRLTSLYAEHPGDCLIFKATSPSGSKMQLLETEFSHTVGELIEVHLRRQDSIPAFLSSLTLELFSRQTVA

SEQ ID NO: 24 - protein, 205 aa, Homo sapiens
MALQLSREQGITLRGSAEIVAEFFSFGINSILYQRGIYPSETFTRVQKYGLTLLVTTDLELIKYLNNVVEQLKDWLYKCSVQKLVVVISNIESGEVLERWQFDIECDKTAKDDSAPREKSQKAIQDEIRSVIRQITATVTFLPLLEVSCSFDLLIYTDKDLVVPEKWEESGPQFITNSEEVRLRSFTTTIHKVNSMVAYKIPVND

SEQ ID NO: 25 - protein, 274 aa, Homo sapiens
MAAPEAEVLSSAAVPDLEWYEKSEETHASQIELLETSSTQEPLNASEAFCPRDCMVPVVFPGPVSQEGCCQFTCELLKHIMYQRQQLPLPYEQLKHFYRKPSPQAEEMLKKKPRATTEVSSRKCQQALAELESVLSHLEDFFARTLVPRVLILLGGNALSPKEFYELDLSLLAPYSVDQSLSTAACLRRLFRAIFMADAFSELQAPPLMGTVVMAQGHRNCGEDWFRPKLNYRVPSRGHKLTVTLSCGRPSIRTTAWEDYIWFQAPVTFKGFRE

SEQ ID NO: 26 - protein, 330 aa, Homo sapiens
MSDSEKLNLDSIIGRLLEVQGSRPGKNVQLTENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVQGWGENDRGVSFTFGAEVVAKFLHKHDLDLICRAHQVVEDGYEFFAKRQLVTLFSAPNYCGEFDNAGAMMSVDETLMCSFQILKPADKNKGKYGQFSGLNPGGRPITPPRNSAKAKK

SEQ ID NO: 27 - protein, 327 aa, Homo sapiens
MADGELNVDSLITRLLEVRGCRPGKIVQMTEAEVRGLCIKSREIFLSQPILLELEAPLKICGDIHGQYTDLLRLFEYGGFPPEANYLFLGDYVDRGKQSLETICLLLAYKIKYPENFFLLRGNHECASINRIYGFYDECKRRFNIKLWKTFTDCFNCLPIAAIVDEKIFCCHGGLSPDLQSMEQIRRIMRPTDVPDTGLLCDLLWSDPDKDVQGWGENDRGVSFTFGADVVSKFLNRHDLDLICRAHQVVEDGYEFFAKRQLVTLFSAPNYCGEFDNAGGMMSVDETLMCSFQILKPSEKKAKYQYGGLNSGRPVTPPRTANPPKKR

SEQ ID NO: 28 - protein, 323 aa, Homo sapiens
MADLDKLNIDSIIQRLLEVRGSKPGKNVQLQENEIRGLCLKSREIFLSQPILLELEAPLKICGDIHGQYYDLLRLFEYGGFPPESNYLFLGDYVDRGKQSLETICLLLAYKIKYPENFFLLRGNHECASINRIYGFYDECKRRYNIKLWKTFTDCFNCLPIAAIVDEKIFCCHGGLSPDLQSMEQIRRIMRPTDVPDQGLLCDLLWSDPDKDVLGWGENDRGVSFTFGAEVVAKFLHKHDLDLICRAHQVVEDGYEFFAKRQLVTLFSAPNYCGEFDNAGAMMSVDETLMCSFQILKPAEKKKPNATRPVTPPRGMITKQAKK

SEQ ID NO: 29 - protein, 432 aa, Homo sapiens
MDEAVGDLKQALPCVAESPTVHVEVHQRGSSTAKKEDINLSVRKLLNRHNIVFGDYTWTEFDEPFLTRNVQSVSIIDTELKVKDSQPIDLSACTVALHIFQLNEDGPSSENLEEETENIIAANHWVLPAAEFHGLWDSLVYDVEVKSHLLDYVMTTLLFSDKNVNSNLITWNRVVLLHGPPGTGKTSLCKALAQKLTIRLSSRYRYGQLIEINSHSLFSKWFSESGKLVTKMFQKIQDLIDDKDALVFVLIDEVESLTAARNACRAGTEPSDAIRVVNAVLTQIDQIKRHSNVVILTTSNITEKIDVAFVDRADIKQYIGPPSAAAIFKIYLSCLEELMKCQIIYPRQQLLTLRELEMIGFIENNVSKLSLLLNDISRKSEGLSGRVLRKLPFLAHALYVQAPTVTIEGFLQALSLAVDKQFEERKKLAAYI

SEQ ID NO: 30 - protein, 2363 aa, Homo sapiens
MAAVLQQVLERTELNKLPKSVQNKLEKFLADQQSEIDGLKGRHEKFKVESEQQYFEIEKRLSHSQERLVNETRECQSLRLELEKLNNQLKALTEKNKELEIAQDRNIAIQSQFTRTKEELEAEKRDLIRTNERLSQELEYLTEDVKRLNEKLKESNTTKGELQLKLDELQASDVSVKYREKRLEQEKELLHSQNTWLNTELKTKTDELLALGREKGNEILELKCNLENKKEEVSRLEEQMNGLKTSNEHLQKHVEDLLTKLKEAKEQQASMEEKFHNELNAHIKLSNLYKSAADDSEAKSNELTRAVEELHKLLKEAGEANKAIQDHLLEVEQSKDQMEKEMLEKIGRLEKELENANDLLSATKRKGAILSEEELAAMSPTAAAVAKIVKPGMKLTELYNAYVETQDQLLLEKLENKRINKYLDEIVKEVEAKAPILKRQREEYERAQKAVASLSVKLEQAMKEIQRLQEDTDKANKQSSVLERDNRRMEIQVKDLSQQIRVLLMELEEARGNHVIRDEEVSSADISSSSEVISQHLVSYRNIEELQQQNQRLLVALRELGETREREEQETTSSKITELQLKLESALTELEQLRKSRQHQMQLVDSIVRQRDMYRILLSQTTGVAIPLHASSLDDVSLASTPKRPSTSQTVSTPAPVPVIESTEAIEAKAALKQLQEIFENYKKEKAENEKIQNEQLEKLQEQVTDLRSQNTKISTQLDFASKRYEMLQDNVEGYRREITSLHERNQKLTATTQKQEQIINTMTQDLRGANEKLAVAEVRAENLKKEKEMLKLSEVRLSQQRESLLAEQRGQNLLLTNLQTIQGILERSETETKQRLSSQIEKLEHEISHLKKKLENEVEQRHTLTRNLDVQLLDTKRQLDTETNLHLNTKELLKNAQKEIATLKQHLSNMEVQVASQSSQRTGKGQPSNKEDVDDLVSQLRQTEEQVNDLKERLKTSTSNVEQYQAMVTSLEESLNKEKQVTEEVRKNIEVRLKESAEFQTQLEKKLMEVEKEKQELQDDKRRAIESMEQQLSELKKTLSSVQNEVQEALQRASTALSNEQQARRDCQEQAKIAVEAQNKYERELMLHAADVEALQAAKEQVSKMASVRQHLEETTQKAESQLLECKASWEERERMLKDEVSKCVCRCEDLEKQNRLLHDQIEKLSDKVVASVKEGVQGPLNVSLSEEGKSQEQILEILRFIRREKEIAETRFEVAQVESLRYRQRVELLERELQELQDSLNAEREKVQVTAKTMAQHEELMKKTETMNVVMETNKMLREEKERLEQDLQQMQAKVRKLELDILPLQEANAELSEKSGMLQAEKKLLEEDVKRWKARNQHLVSQQKDPDTEEYRKLLSEKEVHTKRIQQLTEEIGRLKAEIARSNASLTNNQNLIQSLKEDLNKVRTEKETIQKDLDAKIIDIQEKVKTITQVKKIGRRYKTQYEELKAQQDKVMETSAQSSGDHQEQHVSVQEMQELKETLNQAETKSKSLESQVENLQKTLSEKETEARNLQEQTVQLQSELSRLRQDLQDRTTQEEQLRQQITEKEEKTRKAIVAAKSKIAHLAGVKDQLTKENEELKQRNGALDQQKDELDVRITALKSQYEGRISRLERELREHQERHLEQRDEPQEPSNKVPEQQRQITLKTTPASGERGIASTSDPPTANIKPTPVVSTPSKVTAAAMAGNKSTPRASIRPMVTPATVTNPTTTPTATVMPTTQVESQEAMQSEGPVEHVPVFGSTSGSVRSTSPNVQPSISQPILTVQQQTQATAFVQPTQQSHPQIEPANQELSSNIVEVVQSSPVERPSTSTAVFGTVSATPSSSLPKRTREEEEDSTIEASDQVSDDTVEMPLPKKLKSVTPVGTEEEVMAEESTDGEVETQVYNQDSQDSIGEGVTQGDYTPMEDSEETSQSLQIDLGPLQSDQQTTTSSQDGQGKGDDVIVIDSDDEEEDDDENDGEHEDYEEDEEDDDDDEDDTGMGDEGEDSNEGTGSADGNDGYEADDAEGGDGTDPGTETEESMGGGEGNHRAADSQNSGEGNTGAAESSFSQEVSREQQPSSASERQAPRAPQSPRRPPHPLPPRLTIHAPPQELGPPVQRIQMTRRQSVGRGLQLTPGIGGMQQHFFDDEDRTVPSTPTLVVPHRTDGFAEAIHSPQVAGVPRFRFGPPEDMPQTSSSHSDLGQLASQGGLGMYETPLFLAHEEESGGRSVPTTPLQVAAPVTVFTESTTSDASEHASQSVPMVTTSTGTLSTTNETATGDDGDEVFVEAESEGISSEAGLEIDSQQEEEPVQASDESDLPSTSQDPPSSSSVDTSSSQPKPFRRVRLQTTLRQGVRGRQFNRQRGVSHAMGGRGGINRGNIN

SEQ ID NO: 31 - protein, 712 aa, Homo sapiens
MLAMDTCKHVGQLQLAQDHSSLNPQKWHCVDCNTTESIWACLSCSHVACGRYIEEHALKHFQESSHPVALEVNEMYVFCYLCDDYVLNDNTTGDLKLLRRTLSAIKSQNYHCTTRSGRFLRSMGTGDDSYFLHDGAQSLLQSEDQLYTALWHRRRILMGKIFRTWFEQSPIGRKKQEEPFQEKIVVKREVKKRRQELEYQVKAELESMPPRKSLRLQGLAQSTIIEIVSVQVPAQTPASPAKDKVLSTSENEISQKVSDSSVKRRPIVTPGVTGLRNLGNTCYMNSVLQVLSHLLIFRQCFLKLDLNQWLAMTASEKTRSCKHPPVTDTVVYQMNECQEKDTGFVCSRQSSLSSGLSGGASKGRKMELIQPKEPTSQYISLCHELHTLFQVMWSGKWALVSPFAMLHSVWRLIPAFRGYAQQDAQEFLCELLDKIQRELETTGTSLPALIPTSQRKLIKQVLNVVNNIFHGQLLSQVTCLACDNKSNTIEPFWDLSLEFPERYQCSGKDIASQPCLVTEMLAKFTETEALEGKIYVCDQCNSKRRRFSSKPVVLTEAQKQLMICHLPQVLRLHLKRFRWSGRNNREKIGVHVGFEEILNMEPYCCRETLKSLRPECFIYDLSAVVMHHGKGFGSGHYTAYCYNSEGGFWVHCNDSKLSMCTMDEVCKAQAYILFYTQRVTENGHSKLLPPELLLGSQHPNEDADTSSNEILS

SEQ ID NO: 32 - protein, 494 aa, Homo sapiens
MGRKKKKQLKPWCWYCNRDFDDEKILIQHQKAKHFKCHICHKKLYTGPGLAIHCMQVHKETIDAVPNAIPGRTDIELEIYGMEGIPEKDMDERRRLLEQKTQESQKKKQQDDSDEYDDDDSAASTSFQPQPVQPQQGYIPPMAQPGLPPVPGAPGMPPGIPPLMPGVPPLMPGMPPVMPGMPPGLHHQRKYTQSFCGENIMMPMGGMMPPGPGIPPLMPGMPPGMPPPVPRPGIPPMTQAQAVSAPGILNRPPAPTATVPAPQPPVTKPLFPSAGQMGTPVTSSSTASSNSESLSASSKALFPSTAQAQAAVQGPVGTDFKPLNSTPATTTEPPKPTFPAYTQSTASTTSTTNSTAAKPAASITSKPATLTTTSATSKLIHPDEDISLEERRAQLPKYQRNLPRPGQAPIGNPPVGPIGGMMPPQPGIPQQQGMRPPMPPHGQYGGHHQGMPGYLPGAMPPYGQGPPMVPPYQGGPPRPPMGMRPPVMSQGGRY

SEQ ID NO: 33 - protein, 779 aa, Homo sapiens
MASFVTEVLAHSGRLEKEDLGTRISRLTRRVEEIKGEVCNMISKKYSEFLPSMQSAQGLITQVDKLSEDIDLLKSRIESEVRRDLHVSTGEFTDLKQQLERDSVVLSLLKQLQEFSTAIEEYNCALTEKKYVTGAQRLEEAQKCLKLLKSRKCFDLKILKSLSMELTIQKQNILYHLGEEWQKLIVWKFPPSKDTSSLESYLQTELHLYTEQSHKEEKTPMPPISSVLLAFSVLGELHSKLKSFGQMLLKYILRPLASCPSLHAVIESQPNIVIIRFESIMTNLEYPSPSEVFTKIRLVLEVLQKQLLDLPLDTDLENEKTSTVPLAEMLGDMIWEDLSECLIKNCLVYSIPTNSSKLQQYEEIIQSTEEFENALKEMRFLKGDTTDLLKYARNINSHFANKKCQDVIVAARNLMTSEIHNTVKIIPDSKINVPELPTPDEDNKLEVQKVSNTQYHEVMNLEPENTLDQHSFSLPTCRISESVKKLMELAYQTLLEATTSSDQCAVQLFYSVRNIFHLFHDVVPTYHKENLQKLPQLAAIHHNNCMYIAHHLLTLGHQFRLRLAPILCDGTATFVDLVPGFRRLGTECFLAQMRAQKGELLERLSSARNFSNMDDEENYSAASKAVRQVLHQLKRLGIVWQDVLPVNIYCKAMGTLLNTAISEVIGKITALEDISTEDGDRLYSLCKTVMDEGPQVFAPLSEESKNKKYQEEVPVYVPKWMPFKELMMMLQASLQEIGDRWADGKGPLAAAFSSSEVKALIRALFQNTERRAAALAKIK

SEQ ID NO: 34 - protein, 591 aa, Homo sapiens
MWERLNCAAEDFYSRLLQKFNEEKKGIRKDPFLYEADVQVQLISKGQPNPLKNILNENDIVFIVEKVPLEKEETSHIEELQSEETAISDFSTGENVGPLALPVGKARQLIGLYTMAHNPNMTHLKINLPVTALPPLWVRCDSSDPEGTCWLGAELITTNNSITGIVLYVVSCKADKNYSVNLENLKNLHKKRHHLSTVTSKGFAQYELFKSSALDDTITASQTAIALDISWSPVDEILQIPPLSSTATLNIKVESGEPRGPLNHLYRELKFLLVLADGLRTGVTEWLEPLEAKSAVELVQEFLNDLNKLDGFGDSTKKDTEVETLKHDTAAVDRSVKRLFKVRSDLDFAEQLWCKMSSSVISYQDLVKCFTLIIQSLQRGDIQPWLHSGSNSLLSKLIHQSYHGTMDTVSLSGTIPVQMLLEIGLDKLKKDYISFFIGQELASLNHLEYFIAPSVDIQEQVYRVQKLHHILEILVSCMPFIKSQHELLFSLTQICIKYYKQNPLDEQHIFQLPVRPTAVKNLYQSEKPQKWRVEIYSGQKKIKTVWQLSDSSPIDHLNFHKPDFSELTLNGSLEERIFFTNMVTCSQVHFK

SEQ ID NO: 35 - RNA, 20 nt, synthetic construct
  source 1..20: mol_type = other RNA
aacatgtatgtgtgaagcat

SEQ ID NO: 36 - RNA, 20 nt, synthetic construct
  source 1..20: mol_type = other RNA
gtcacagaagtctctaccaa

SEQ ID NO: 37 - RNA, 20 nt, synthetic construct
  source 1..20: mol_type = other RNA
gtcagaccaaaatacctact

SEQ ID NO: 38 - RNA, 20 nt, synthetic construct
  source 1..20: mol_type = other RNA
gaggccaagaacagctcttg

SEQ ID NO: 39 - RNA, 20 nt, synthetic construct
  source 1..20: mol_type = other RNA
tggaaaagactatgaccctg

SEQ ID NO: 40 - RNA, 20 nt, synthetic construct
  source 1..20: mol_type = other RNA
ctcaacacagagaaccgctg

SEQ ID NO: 41 - RNA, 20 nt, synthetic construct
  source 1..20: mol_type = other RNA
actggaggggcatttagcca

SEQ ID NO: 42 - RNA, 20 nt, synthetic construct
  source 1..20: mol_type = other RNA
gcatactggatagccatcca

SEQ ID NO: 43 - RNA, 20 nt, synthetic construct
  source 1..20: mol_type = other RNA
gaagaagcgcgagacccacg

SEQ ID NO: 44 - RNA, 20 nt, synthetic construct
  source 1..20: mol_type = other RNA
gctggacctgcaacacaagt

SEQ ID NO: 45 - RNA, 20 nt, synthetic construct
  source 1..20: mol_type = other RNA
aagctaacgatgaaaatcgg

SEQ ID NO: 46 - RNA, 20 nt, synthetic construct
  source 1..20: mol_type = other RNA
aaacattcggaacactatgg

SEQ ID NO: 47 - RNA, 20 nt, synthetic construct
  source 1..20: mol_type = other RNA
cgagtcgccaacggtccacg

SEQ ID NO: 48 - RNA, 20 nt, synthetic construct
  source 1..20: mol_type = other RNA
ttgtgtttggtgattacaca

SEQ ID NO: 49 - RNA, 20 nt, synthetic construct
  source 1..20: mol_type = other RNA
aactcaccagcagtacgtgt

SEQ ID NO: 50 - RNA, 20 nt, synthetic construct
  source 1..20: mol_type = other RNA
catcaaggtctttcccaacg

SEQ ID NO: 51 - RNA, 19 nt, synthetic construct
  source 1..19: mol_type = other RNA
acaaatccaggtcccagtg

SEQ ID NO: 52 - RNA, 19 nt, synthetic construct
  source 1..19: mol_type = other RNA
tatctctggagctaaagcg

SEQ ID NO: 53 - RNA, 19 nt, synthetic construct
  source 1..19: mol_type = other RNA
tatgagtaaactttctggc

SEQ ID NO: 54 - RNA, 19 nt, synthetic construct
  source 1..19: mol_type = other RNA
agtccatctcctatgtcct

SEQ ID NO: 55 - RNA, 19 nt, synthetic construct
  source 1..19: mol_type = other RNA
aactgattcatcaagagag

SEQ ID NO: 56 - RNA, 19 nt, synthetic construct
  source 1..19: mol_type = other RNA
tacaatggaatacagattg

SEQ ID NO: 57 - RNA, 19 nt, synthetic construct
  source 1..19: mol_type = other RNA
tttcctgcacaaacttggt

SEQ ID NO: 58 - DNA, 41 bp, synthetic construct
  source 1..41: mol_type = other DNA
aatggactatcatatgcttaccgtaacttgaaagtatttcg

SEQ ID NO: 59 - DNA, 44 bp, synthetic construct
  source 1..44: mol_type = other DNA
ctttagtttgtatgtctgttgctattatgtctactattctttcc

SEQ ID NO: 60 - DNA, 58 bp, synthetic construct
  source 1..58: mol_type = other DNA
aatgatacggcgaccaccgagatctacactctttccctacacgacgctcttccgatct

SEQ ID NO: 61 - DNA, 24 bp, synthetic construct
  source 1..24: mol_type = other DNA
tcttgtggaaaggacgaaacaccg

SEQ ID NO: 62 - DNA, 83 bp, synthetic construct
  source 1..83: mol_type = other DNA
caagcagaagacggcatacgagatgtgactggagttcagacgtgtgctcttccgatcttctactattctttcccctgcactgt

(19) United States
(12) Patent Application Publication
(10) Pub. No.: US 2025/0325527 A1
(43) Pub. Date: Oct. 23, 2025

(54) KIF18A INHIBITION FOR TREATMENT OF CANCER

(71) Applicants: AMGEN INC., Thousand Oaks, CA (US); The Johns Hopkins University, Baltimore, MD (US)

(72) Inventors: Marc Noel Payton, Newbury Park, CA (US); Andrew Holland, Baltimore, MD (US); Colin Richard Gliech, Baltimore, MD (US); Peter Yeow, Baltimore, MD (US)

(21) Appl. No.: 18/849,825

(22) PCT Filed: Apr. 28, 2023
(86) PCT No.: PCT/US23/66387

§ 371 Date: Sep. 23, 2024

Related U.S. Application Data
(60) Provisional application No. 63/336,731, filed on Apr. 29, 2022.

(51) Int. Cl. A61K 31/438; A61K 31/506; A61P 35/00; C12Q 1/6886

(52) U.S. Cl. CPC A61K 31/438; A61K 31/506; A61P 35/00; C12Q 1/6886; C12Q 2600/106; C12Q 2600/156

(57) ABSTRACT

Provided herein are methods of determining a treatment for a subject having a neoplastic disease, said method comprising assaying a sample obtained from the subject for (a) SAC activity, (b) ploidy, (c) WGD, (d) APC/C activity, or (e) a combination thereof. In exemplary embodiments, the treatment determined for the subject comprises, consists essentially of, or consists of a KIF18A inhibitor, when the sample is positive for (a) increased SAC activity, (b) high ploidy, (c) WGD, (d) low APC/C activity, (d) or a combination thereof.

### CROSS REFERENCE TO RELATED APPLICATION

The benefit under 35 U.S.C. § 119 (e) of U.S. Provisional Application No. 63/336,731, filed Apr. 29, 2022, is hereby claimed, and the entire disclosure of this application is incorporated herein by reference.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 71.7 KB XML file named “10144-WP01-SEC.xml”; created on Apr. 24, 2023.

### BACKGROUND

Cancer is caused by dysregulated cellular proliferation, making the cell cycle an appealing target for therapeutics. Early successes with this approach include the mitotic inhibitor, Paclitaxel, which is highly efficacious and still widely used in the clinic today. However, broad inhibition of proliferation universally affects cycling tissues. As a result, nearly all other drugs targeting core cell cycle machinery have failed clinically due to high cytotoxicity. Identifying synthetic vulnerabilities in the cancer cell cycle may therefore prove important for the success of ongoing drug development. Recently, the mitotic kinesin KIF18A has been identified as essential for the division of a subset of whole genome doubled (WGD) cancers (Quinton et al., Nature 590:492-497 (2021); Marquis et al., Nature Communications 12, Article no. 1213 (2021)). KIF18A is dispensable for normal cellular division and KIF18A−/− mice are fully viable. It remains unclear however what causes KIF18A dependency and there are currently no known biomarkers for targeting patient populations. Thus, there is a need for identifying the determinants of KIF18A dependency to better identify targetable tumors.

### SUMMARY

Presented herein are data evidencing biomarkers of sensitivity to KIF18A inhibitor treatment. Accordingly, the present disclosure provides a method of determining a treatment for a subject having a neoplastic disease, said method comprising assaying a sample obtained from the subject for (a) Spindle Assembly Checkpoint (SAC) activity, (b) ploidy (c) whole genome doubling (WGD), (d) Anaphase Promoting Complex (APC/C) activity, or (e) a combination thereof. In exemplary embodiments, the treatment determined for the subject comprises, consists essentially of, or consists of a KIF18A inhibitor, when the sample is positive for (a) increased SAC signaling or SAC activity, (b) high ploidy, (c) WGD, (d) low APC/C activity, (d) or a combination thereof. The present disclosure also provides a method of treating a subject having a neoplastic disease. In exemplary embodiments, the method comprises (I) assaying a sample obtained from the subject for (a) SAC activity, (b) ploidy (c) whole genome doubling (WGD), (d) Anaphase Promoting Complex (APC/C) activity, or (e) a combination thereof and (II) administering a KIF18A inhibitor to the subject when the sample is positive for (a) increased SAC signaling or SAC activity, (b) high ploidy, (c) WGD, (d) low APC/C activity, (e) or a combination thereof as assayed in (I), optionally, wherein the method further comprises obtaining the sample from the subject. The present disclosure additionally provides methods of treating a subject having a neoplastic disease, wherein the subject comprises cells that are positive for (a) increased SAC signaling or SAC activity, (b) high ploidy, (c) WGD, (d) low APC/C activity, (e) or a combination thereof. In exemplary embodiments, the method comprises administering a KIF18A inhibitor to the subject. The present disclosure further provides a method of identifying a subject having a neoplastic disease as sensitive to treatment with a KIF18A inhibitor. In exemplary embodiments, the method comprises assaying a sample obtained from the subject for (a) SAC activity, (b) ploidy (c) WGD, (d) APC/C activity, or (e) a combination thereof, wherein the subject is identified as sensitive to treatment with a KIF18A inhibitor, when the sample is positive for (a) increased SAC signaling or SAC activity, (b) high ploidy, (c) WGD, (d) low APC/C activity, (e) or a combination thereof. The present disclosure provides a method of treating a subject with a cancer comprising one or more whole genome duplication or whole genome doubling (WGD) events, said method comprising: (a) assaying APC/C activity in a tumor cell obtained from the subject; and (b) administering to the subject a KIF18A inhibitor when the APC/C activity measured in (a) is low. Further provided is a method of treating a subject with a cancer comprising one or more whole genome duplication or whole genome doubling (WGD) events, said method comprising (a) lowering APC/C activity in the subject, optionally, by inhibiting expression of UBE2S; and (b) administering to the subject a KIF18A inhibitor. Provided herein is a method of treating a subject with a cancer comprising one or more whole genome duplication or whole genome doubling (WGD) events. In exemplary embodiments, the method comprises (a) administering to the subject an agent that lowers APC/C activity in the subject; and (b) administering to the subject a KIF18A inhibitor. In exemplary aspects, the assaying step comprises assaying the sample for expression levels of RNA or protein encoded by one or more of the following genes: ANAPC1, ANAPC2, ANAPC4, ANAPC5, ANAPC7, ANAPC10, ANAPC11, ANAPC13, ANAPC15, ANAPC16, CDC16, CDC23, CDC26, CDC27, UBE2C, UBE2D1, and UBE2S. In exemplary instances, the assaying step comprises assaying the sample for assaying expression levels of RNA or protein encoded by one or more of the following genes: BUB1, BUB1B, BUB3, AURKB, CCNB1, MAD1L1, MAD2L1, MAD2L1GP, PPP1CA, PPP1CB, PPP1CC, TRIP13, TPR, USP44, ZNF207, ZW10, and ZWILCH. Optionally, the assaying step comprises measuring ploidy and/or WGD via chromosome counting (via e.g., karyotyping, parallel sequencing, comparative genomic hybridization (CGH), microarrays) high throughput sequencing (HTS), or flow cytometry. In various aspects, the sample comprises cancer cells, tumor cells, non-tumor cells, blood, blood cells, or plasma, optionally, wherein the sample comprises germline cancer cells or somatic cancer cells. In various instances, the neoplastic disease is a cancer, optionally, breast cancer, ovarian cancer, endometrial cancer, lung cancer, or prostate cancer. In exemplary aspects, the neoplastic disease is triple-negative breast cancer (TNBC), non-luminal breast cancer, high-grade serous ovarian cancer (HGSOC), endometrial cancer, optionally, serous endometrial cancer, or non-small-cell lung cancer. The sample, in various aspects, is positive for one or more whole genome duplication or whole genome doubling (WGD) events. In various aspects, treatment with or administration of the KIF18A inhibitor induces at least 50% (e.g., at least 75%, at least 80% or 85%, at least 90% or 95%) tumor regression, compared to a control. In exemplary instances, the KIF18A inhibitor is Compound C9, which is 4-(N-(tert-butyl) sulfamoyl)-N-(3-(N-(tert-butyl) sulfamoyl)phenyl)-2-(6-azaspiro[2.5]octan-6-yl)benzamide and/or has the following structure:

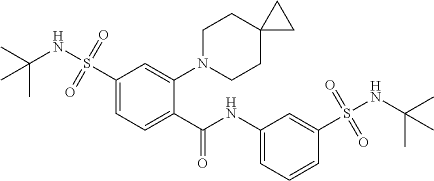 [SMILES: CC(C)(C)NS(=O)(=O)c1cccc(NC(=O)c2ccc(S(=O)(=O)NC(C)(C)C)cc2N2CCC3(CC2)CC3)c1]

or is N-(2-(4,4-Difluoropiperidin-1-yl)-6-methylpyrimidin-4-yl)-4-((2-hydroxyethyl) sulfonamido)-2-(6-azaspiro[2.5]octan-6-yl)benzamide and/or has the following structure:

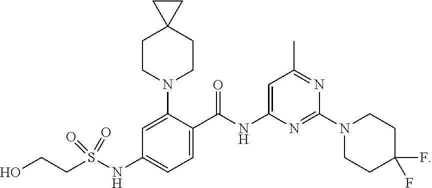 [SMILES: Cc1cc(NC(=O)c2ccc(NS(=O)(=O)CCO)cc2N2CCC3(CC2)CC3)nc(N2CCC(F)(F)CC2)n1]

In various aspects, the KIF18A inhibitor is administered for oral administration, optionally once a day.

### BRIEF DESCRIPTION OF THE DRAWINGS

FIG. 1 is a series of scatter plots of pooled barcoded cancer cell lines treated in a 5-day cell growth assay with DMSO or KIF18Ai C9 (8-point concentration range), relative abundance of unique cell line barcodes was measured to estimate cell viability. KIF18Ai C9 AUC values versus CIN features (whole genome doubling (WGD), ploidy, aneuploidy score (AS)) plus TP53 status in breast and ovarian cancer cell lines (n=58). Scatter plots show KIF18Ai C9 AUC versus CIN features plus TP53 status with mean AUC indicated for each group. Colored cell lines overlap with KIF18Ai C9 sensitivity in nonbarcoded screens. Dotted line indicates AUC value of 0.65. ** p=0.0037, *** p=0.0004 by unpaired t-test.

FIG. 2A is a graph of 5-day cell viability plotted as a function of KIF18Ai concentration. Example KIF18Ai drug titrations are shown. FIG. 2B is an image of the immunofluorescence of KIF18A localization in metaphase OVCAR-3 cells. FIG. 2C is a graph showing the top hits in KIF18Ai CRISPR-Cas9 whole genome knockout screens. FIG. 2D is a graph of mitotic duration and outcome of KIF18Ai. FIG. 2E is a graph of % rescue of KIF18Ai toxicity with partial inhibition of MSP1 across sensitive cell lines.

FIGS. 3A-3B show spindle assembly checkpoint activation with KIF18Ai. FIG. 3A is a series of immunofluorescence images of cells held in metaphase using proteasome inhibitor MG132. DMSO condition indicates normal SAC silencing, whereas microtubule poison Nocodazole leads to maximal SAC activation. FIG. 3B is a pair of graphs which represent quantification of the images in FIG. 3A.

FIG. 4 is a graph of viability and supports expression of a drug resistant transgene rescues cell viability independently of CDK1/PP1 recruitment to kinetochores.

FIG. 5A is a graph KIF18Ai toxicity vs. modal chromosome number and FIG. 5B is a pair of graphs showing the effect of WGD on KIF18Ai toxicity.

FIG. 6A is a graph showing an increase in metaphase to anaphase transition duration in untreated cells. FIG. 6B is a graph from 7-day growth assays of RPE1 diploid and tetraploid cells with APC4 knockdown and western blot confirmation (similar shUBE2S results not shown). FIG. 6C is a graph showing the rescue of viability in sensitive HCC-1806 by increasing APC/C activity using UBE2S overexpression.

FIG. 7 is a schematic of sensitivity to KIF18Ai.

FIGS. 8A-8H support that sensitivity to KIF18A inhibition is defined by long mitotic delays that drive catastrophic errors. FIG. 8A is a widefield immunofluorescence image of KIF18A (bottom inset) localization in DMSO or KIF18Ai treated OVCAR-3. Kinetochores (CENPA, lightest gray, top inset) and the mitotic spindle (α-Tubulin, medium gray). Scale bar=5 μm. FIG. 8B is a titration curve of KIF18Ai in 5-day MTT endpoint viability assay for DLD1 and OVCAR-3 cells. Data are represented as mean±SD. N=3 technical replicates from a single experiment. FIG. 8C is a summary table of KIF18Ai toxicity and IC50 values from MTT assays. Toxicity values are derived from plateau measurements of the IC50 dose-response curves. FIG. 8D is a titration curve of KIF18Ai in 5-day MTT endpoint viability assay for HCC1806 cells constitutively expressing a WT or drug-resistant (G289I) KIF18A-(3×) HA transgene. Data are represented as mean±SD. N=3 technical replicates from a single experiment. FIG. 8E is a quantification of live-cell widefield timelapse microscopy of H2B/α-Tubulin fluorescently tagged cell lines colored by mitotic outcome. Data are represented as mean±SD. Statistical significance was determined using an unpaired two-tailed Student's t-test. FIG. 8F is a quantification of micronucleus formation after 7 days of DMSO or KIF18Ai treatment in indicated cell lines. γH2A.X+ micronuclei denote DNA damage and likely micronucleus rupture. Data are represented as mean±SD. N=5 independent experiments, n>=100 cells per condition per experiment. Statistical significance was determined using an unpaired two-tailed Student's t-test. FIG. 8G shows scDNAseq of indicated cell lines following 7 days of DMSO or KIF18Ai treatment. Asterisk indicates common karyotypic alterations in each cell line. FIG. 8H is a series of graphs of heterogeneity score calculations for each sample in FIG. 8F.

FIGS. 9A-9J support that SAC Activation drives KIF18Ai Toxicity. FIG. 9A is a comparison of OVCAR-3 and HCC1806 whole genome CRISPR-Cas9 screens. Data is annotated with mean±1.5×SD. Hits with an average FDR <0.3 are highlighted in darker gray. FIG. 9B is a gene set enrichment analysis of genes from FIG. 9A with a β(KIF18Ai-DMSO)>1.5 SD and an average FDR <0.3. FIG. 9C is a 5-day MTT endpoint viability assay of HCC1806 polyclonal CRISPR-Cas9 KO cell lines in KIF18Ai. Data are represented as mean±SD. N=3 independent experiments, n=3 technical replicates per experiment. Statistical significance was determined using a one-way-ANOVA with post-hoc Dunnett's multiple comparisons test between each gene knockout pair and WT. FIG. 9D is a quantification of mitotic duration and fate of mitotic HCC1806 monoclonal cell lines arrested with DMN. Data are represented as mean±SD. Statistical significance was determined using a one-way-ANOVA with post-hoc Dunnett's multiple comparisons test between each sample and WT. Scale bar=10 μm. FIG. 9E, KIF18Ai viability rescue across sensitive cell line panel with sub-saturating Reversine treatment (30 nM) in a 5-day MTT endpoint assay. Data are represented as mean±SD. N=3 independent experiments, n=3 technical replicates per experiment. Statistical significance was determined using a one sample Student's t-test, HO: % rescue=0. FIG. 9F is a quantification of BUBR1 intensity at kinetochores across HCC1806 clonal rescue cell lines in DMSO, KIF18Ai, and Nocodazole. Violin plots summarize all individual kinetochores analyzed, points represent per-cell intensity averages, and error bars are mean±SD of per-cell averages. Statistical significance was determined using a one-way-ANOVA with post-hoc Dunnett's multiple comparisons test between KIF18Ai intensity in edited cell lines and KIF18Ai intensity in WT. FIG. 9G is a quantification of MAD1 intensity at kinetochores as in FIG. 9F. Statistical significance was determined using a one-way-ANOVA with post-hoc Dunnett's multiple comparisons test between KIF18Ai intensity in edited cell lines and KIF18Ai intensity in WT. FIG. 9H is a set of wide-field immunofluorescence images of SAC proteins BUBR1 and MAD1 at kinetochores in DMSO, KIF18Ai, and Nocodazole treatments in mitotic HCC1806 cells. Scale bar=5 μm. FIG. 9I, 5-day MTT endpoint viability assay of HCC1806 G289I KIF18A mutant overexpression cell lines in KIF18Ai. Data are represented as mean±SD. N=3 independent experiments, n=3 technical replicates per experiment. Statistical significance was determined using a one-way-ANOVA with post-hoc Dunnett's multiple comparisons test between each sample and G289I KIF18A (WT) overexpression. FIG. 9J is a titration of CDK1 inhibitor RO-3306 in a 5-day MTT endpoint viability assay against DMSO or KIF18Ai treated HCC1806 cells. N=3 technical replicates from single experiment. Error bars represent mean±SD

FIGS. 10A1-10I support that KIF18Ai toxicity is relieved by stabilizing kinetochore-microtubule attachments and rescuing metaphase plate congression. FIGS. 10A1 to 10A3 are quantifications of metaphase plate congression and mitotic outcome from live-cell timelapse widefield fluorescent microscopy of dividing HCC1806 monoclonal rescue. FIG. 10B is a graph showing the longest continuous congressed metaphase from movies. Statistical significance was determined using a one-way-ANOVA with post-hoc Dunnett's multiple comparisons test between WT and edited cell lines in KIF18Ai. FIG. 10C is a quantification of MAD1 intensity at kinetochores relative to kinetochore position along the mitotic spindle axis in DMSO (N=35 cells, n=3104 kinetochores) or KIF18Ai (N=36 cells, n=3369 kinetochores) treated HCC1806 cells from wide-field immunofluorescence images dataset from FIG. 4C. MAD1+ is defined as >99th percentile signal intensity of DMSO condition. FIG. 10D is a series of graphs showing Kinetochore distribution probability along the spindle axis from FIG. 10C (FIG. 10D top) and Probability of a kinetochore being MAD1+ relative to its position on the spindle axis in DMSO or KIF18Ai conditions (FIG. 10D bottom). Polar kinetochores are defined as >5 μm from the spindle midline. FIG. 10E is a graph of percent of cells containing at least one MAD1+ kinetochore in either the central (<=5 μm from midline) or polar (>5 μm from midline) regions in DMSO or KIF18Ai conditions from FIG. 10C. FIG. 10F is a series of representative still images from live-cell confocal timelapse movies of dividing H2B/α-Tubulin fluorescently tagged HCC1806 and Hela cells in KIF18Ai. Scale bar=5 μm. FIG. 10G is a set of representative still images from live-cell confocal timelapse movies of HCC1806 PA-GFP-α-Tubulin cells. Scale bar=5 μm. Intensity distribution over time of photo-activated PA-GFP-α-Tubulin band. FIG. 10H is a second-order exponential decay fit of integrated intensity measurements for RPE1 PA-GFP-α-Tubulin cells in DMSO or KIF18Ai. Solid shaded region represents mean±SEM. Dots represent individual measurements. N>35 cells/condition. ANOVA analysis of fit between conditions was not significant. FIG. 10I is a graph of K-MT half-life measurements from 3 h and FIG. 21B. Error bar represents SE.

FIGS. 11A-11D supports that KIF18Ai driven SAC signaling occurs in both sensitive and insensitive cell lines. FIG. 11A is a graph showing intensity of BUBR1 at kinetochores in DMSO, KIF18Ai, and Nocodazole treatments across the full panel of cell lines from wide-field immunofluorescence images. Violin plots summarize all individual kinetochores analyzed, points represent per-cell intensity averages, and error bars are mean±SD of per cell averages. Statistical significance was determined using a one-way-ANOVA with post-hoc Dunnett's multiple comparisons test between drug and DMSO conditions for each cell line. FIG. 11B is a graph showing linear correlation between 5-day KIF18Ai toxicity and BuBR1 defect across cell line panel. squares=sensitive, circles=insensitive cell lines. BUBR1 defect=(Signal [KIF18Ai]−Signal [DMSO])/(Signal [Nocodazole]−Signal [DMSO])×100. FIG. 11C is a graph showing average number of MAD1+kinetochores per cell in DMSO, KIF18Ai, and Nocodazole treatments across the full panel of cell lines from wide-field immunofluorescence images. MAD1+kinetochores have MAD1 signal >nth percentile of signal in DMSO condition as indicated. Statistical significance was determined from >95% MAD1+kinetochores using a one-way-ANOVA with post-hoc Dunnett's multiple comparisons test between drug and DMSO conditions for each cell line. FIG. 11D is a Linear correlation between 5-day KIF18Ai toxicity and number of >95% MAD1+kinetochores in KIF18Ai across cell line panel.

FIGS. 12A-121 supports that whole Genome Doubling synergizes with APC/C defects to induce KIF18A dependency. FIG. 12A is a graph of average number of MAD1+kinetochores per cell in DMSO, KIF18Ai, and Nocodazole treatments between diploid and WGD RPE1 cell lines from wide-field immunofluorescence images. MAD1+kinetochores have MAD1 signal >nth percentile of signal in DMSO condition as indicated. Statistical significance was determined from >95% MAD1+kinetochores using an unpaired two-tailed Student's t-test for each condition between 2N and 4N cells. FIG. 12B is series of graphs from 5-day MTT endpoint viability assay of diploid and WGD cell lines in KIF18Ai. Data are represented as mean±SD. N=5 independent experiments, n=3 technical replicates per experiment. Statistical significance was determined using an unpaired two-tailed Student's t-test. FIG. 12C is a linear correlation between 5-day KIF18Ai toxicity and modal chromosome number for each cell line taken from the ATCC. FIG. 12D is a comparison of OVCAR-8 whole genome CRISPR-Cas9 screens at IC90 and IC50 [KIF18Ai]. Data is annotated with mean±1.5×SD. Hits with average FDR <0.1 are outlined in the central plot and dark gray in the exterior rank plots. FIG. 12E is a gene set enrichment analysis of genes from D with B (KIF18Ai-DMSO)<1.5×SD and average FDR <0.1. FIG. 12F is a series of graphs of longitudinal confluency measurements of OVCAR-8 WT, ΔAPC4 (polyclonal, sgRNA A), and ΔUBE2S (polyclonal, sgRNA A) in DMSO, KIF18Ai, and Nocodazole conditions. N=3 technical replicates from a single experiment. Data are represented as mean±SD. FIG. 12G is a graph from a 5-day MTT endpoint viability assay of OVCAR-8 polyclonal CRISPR-Cas9 KO cell lines in KIF18Ai. Data are represented as mean±SD. N=3 independent experiments, n=3 technical replicates per experiment. Statistical significance was determined using a one-way-ANOVA with post-hoc Dunnett's multiple comparisons test between each gene knockout pair and WT. FIG. 12H is a set of graphs showing longitudinal confluency measurements of shAPC4 and shUBE2S diploid and WGD RPE1 cells in DMSO, KIF18Ai, and Nocodazole conditions. N=3 technical replicates from a single experiment. Data are represented as mean±SD. FIG. 12I is a pair of graphs from a 5-day MTT endpoint viability assay of shAPC4 and shUBE2S diploid and WGD RPE1 cell lines in KIF18Ai. Data are represented as mean±SD. N=3 technical replicates from a single experiment. Statistical significance was determined using a one-way-ANOVA with post-hoc Dunnett's multiple comparisons test between each 2N and 4N knockdown and 2N non-targeting sgRNA cell lines.

FIGS. 13A-13K support that low basal APC/C activity is a hallmark of KIF18A-dependent cell lines. FIG. 13A is a graph showing linear correlation between 5-day KIF18Ai toxicity and mitotic duration in cells without errors from live-cell widefield timelapse microscopy of H2B/α-Tubulin fluorescently tagged cell lines from FIG. 8d. FIG. 13B is a graph showing metaphase to anaphase duration from live-cell widefield timelapse microscopy of H2B/α-Tubulin fluorescently tagged cell lines from FIG. 8D. Error bars represent mean±SD. FIG. 13C is a graph showing linear correlation between metaphase to anaphase duration in FIG. 13B and mitotic duration in KIF18Ai from live-cell widefield timelapse microscopy of H2B/α-Tubulin fluorescently tagged cell lines from FIG. 8d. FIG. 13D (Top) shows KIF18A co-dependency relationships from the DepMap RNAi dataset. N=600 cell lines. FIG. 13E is a live-cell widefield timelapse microscopy image of Cyclin B1-eYFP at the metaphase to anaphase transition from in endogenously tagged HeLa cell lines in response to DMSO or KIF18Ai. Scale bar=10 μm. FIG. 13F is a series of graphs that show quantification of Cyclin B1 degradation rates as in FIG. 13E for endogenously tagged RPE1 and HeLa cell lines in response to DMSO, KIF18Ai, Nocodazole, and Reversine treatments. Data is partitioned between fast (<30 min t1/2) and slow (>30 min t1/2) traces then represented as mean±SD for each population. Average t1/2 values are listed. FIG. 13G is a quantification of mitotic outcomes in DMSO or KIF18Ai from (d). N>=20 cells per condition. FIG. 13H are longitudinal confluency measurements of H2B/α-Tubulin fluorescently tagged HCC1806 WT, or UBE2S-overexpressing cells in DMSO, KIF18Ai, or Nocodazole conditions. N=3 technical replicates from a single experiment. Data are represented as mean±SD. FIG. 13I is a graph from a 5-day MTT endpoint viability assay of H2B/α-Tubulin fluorescently tagged HCC1806 UBE2S-overexpressing cell lines in KIF18Ai. Data are represented as mean±SD. N=3 independent experiments, n=3 technical replicates per experiment. Statistical significance was determined using an unpaired two-tailed Student's t-test between WT and UBE2S-overexpressing cells. FIG. 13J is a quantification of live-cell widefield timelapse microscopy of H2B/α-Tubulin fluorescently tagged HCC1806 WT or UBE2S-overexpressing cell lines in DMSO and KIF18Ai conditions colored by mitotic outcome. Error bars represent mean±SD. Statistical significance was determined using an unpaired two-tailed Student's t-test between WT and UBE2S-overexpressing cells for each condition. FIG. 13K is a titration of the MPS1 inhibitor Reversine in a 5-day MTT endpoint viability assay in DMSO or KIF18Ai treated HCC1806 WT or UBE2S overexpressing cells. Open squares are omitted from the curve fit. N=3 technical replicates from a single experiment. Data are represented as mean±SD.

FIG. 14 is an illustration of a model for KIF18A Dependency. (A) Toxicity in KIF18Ai is the result of mitotic delays and errors. Whether a cell arrests in mitosis depends on three factors (i) the number of kinetochores multiplied by (ii) the amount of elevated SAC activity at each kinetochore and mitigated by (iii) the basal activity of the APC/C. Cells with mitotic delays resulting from these factors are generally sensitive to KIF18Ai. However, toxicity can be rescued by hyperstability of the mitotic spindle apparatus as in with MCF7 cells.

FIGS. 15A-15C2 show an extended analysis of cellular response to KIF18Ai. FIG. 15A is a widefield immunofluorescence of KIF18A (top inset) localization relative to spindle poles (CEP192, lightest gray) and the mitotic spindle (α-Tubulin, dark gray, bottom inset) in response to KIF18Ai treatment across full cell line panel. Scale bar=5 μm. Each of FIGS. 15B1-15B2 is a titration of KIF18Ai in a 5-day MTT endpoint viability assay for the panel of sensitive (FIG. 15B2) and insensitive (FIG. 15B1) cell lines. N=3 technical replicates from a single experiment. Data are represented as mean±SD. FIG. 15C1 is a western blot of KIF18A expression levels across the full panel of sensitive and insensitive cell lines. FIG. 15C2 is a linear correlation between 5-day KIF18Ai toxicity and normalized KIF18A expression (KIF18A/α-Tubulin) from FIG. 15C1. Expression is plotted on a log 2 axis.

FIGS. 16A-16C are validation of drug-resistant KIF18A transgenic HCC1806 cells. FIG. 16A is a schematic of KIF18Ai drug binding site from EM structure (PDB: 5OAM). Medium gray stucture at top: KIF18Ai motor domain with G289 highlighted in darker gray. Light gray structure at bottom: alpha-tubulin/beta-tubulin. Dotted circle represents drug binding pocket. Protein sequence alignment of drug binding pocket between KIF18A, KIF19, and KIF18B. FIG. 16B is a schematic of KIF18A protein domains and mutations. FIG. 16C is a Western blot validation of HCC1806 cells constitutively expressing a WT, drug resistant (G289I), drug resistant and motor dead (G289I , R308A, K311A), or drug resistant and PP1 binding deficient (G289I , V614A, W617A) KIF18A-(3×) HA transgene.

FIGS. 17A and 17B are Live-cell analysis of mitotic outcomes from KIF18Ai Treatment. FIG. 17A is a proportion of mitotic fates across the panel of H2B/α-Tubulin fluorescently tagged cell lines in response to DMSO or KIF18Ai treatment from live-cell widefield timelapse microscopy in FIG. 8e. FIG. 17B is a representation of mitotic error threshold across a panel of H2B/α-Tubulin fluorescently tagged cell lines in response to DMSO or KIF18Ai treatment from live-cell widefield timelapse microscopy in FIG. 8e. Bars represent individual mitotic events. Dotted line represents error threshold.

FIGS. 18A-18F is a validation of OVCAR-3 and HCC1806 CRISPR Cas9 screen hits. FIG. 18A is a schematic of whole-genome CRISPR-Cas9 knockout screen protocol. FIG. 18B is a Western blot validation of polyclonal HCC1806 sgRNA-targeted cell lines for Cyclin B1, HSET, and MAD1.

FIG. 18C is a Western blot validation of clonal HCC1806 sgRNA-targeted cell lines for Cyclin B1, HSET, and MAD1. Arrow denotes the clone used in further experiments. FIG. 18D is a graph from a 5-day MTT endpoint viability assay of HCC1806 clonal CRISPR-Cas9 KO cell lines in KIF18Ai. Data are represented as mean±SD. N=3 technical replicates. Boxed bar denotes the clone used in further experiments. Statistical significance was determined using a one-way-ANOVA with post-hoc Dunnett's multiple comparisons test between each set of clonal edited cells and WT. FIG. 18E is a quantification of live-cell widefield timelapse microscopy of H2B/α-Tubulin fluorescently tagged edited cell lines colored by mitotic outcome. Error bars represent mean±SD. Statistical significance was determined using an unpaired two-tailed Student's t-test. FIG. 18F shows the proportion of mitotic fates across the panel of H2B/α-Tubulin fluorescently tagged HCC1806 rescue cell lines in response to DMSO or KIF18Ai treatment from live-cell widefield timelapse microscopy.

FIGS. 19A-19B are extended SAC KIF18Ai rescue data. FIG. 19A is a titration of MPS1 inhibitor Reversine in a 5-day MTT endpoint viability assay against DMSO or KIF18Ai treated HCC1806 cells. Open squares are omitted from the curve fit. N=3 technical replicates from a single experiment. Data are represented as mean±SD. FIG. 19B is a titration of KIF18Ai in 5-day MTT endpoint viability assay for HeLa MAD1 knockout FRT TetON VSV-MAD1 and Cyclin B1-binding deficient HeLa MAD1 knockout FRT TetON VSV-MAD1 (E52K, E53K, E56K) cell lines. N=3 technical replicates from single experiment. Data are represented as mean±SD.

FIG. 20 shows SAC activation in transgenic HeLa BUBR1-EGFP cell lines. FIG. 20 (Left) is a quantification of BUBR1 foci from live-cell confocal timelapse microscopy of HeLa EGFP-BUBR1 BAC H2B-iRFP cell lines in DMSO, KIF18Ai, and Nocodazole conditions. Error bars represent mean±SD. Statistical significance was determined using an unpaired two-tailed Student's t-test between DMSO and KIF18Ai conditions at 0, 10, 20, 30, and 40 min. FIG. 20 (Right): Representative still images from mitotic movies.

FIGS. 21A-21D provide extended data for photoactivatable GFP-α-Tubulin cell lines. FIG. 21A is a quantification of spindle flux rates for RPE1 and HCC1806 PA-GFP-α-Tubulin cells in DMSO, KIF18Ai, and Taxol conditions. A linear fit is on data between 0-150 s. N>35 cells/condition for DMSO and KIF18Ai, N=5 cells for Taxol. FIG. 21B is a second-order exponential decay fit of integrated intensity measurements of the mitotic spindle after photoactivation from live-cell confocal timelapse movies of HCC1806 PA-GFP-α-Tubulin WT or AHSET cells in DMSO or KIF18Ai treatment. The solid shaded region represents mean±SEM. Dots represent individual measurements. N>50 cells/condition. ANOVA analysis of fit between conditions for each cell line were: HCC1806 p<0.001, HCC1806 AHSET p<0.001. FIG. 21C is a Western blot validation of clonal HCC1806 H2B-iRFP PA-GFP-α-Tubulin HSET knockout cell lines. Arrow denotes preferred clone. FIG. 21D is a graph from a 5-day MTT endpoint viability assay of clonal HCC1806 H2B-iRFP PA-GFP-α-Tubulin HSET knockout cell lines in KIF18Ai. Data are represented as mean±SD. N=3 technical replicates from single experiment. Boxed bar denotes preferred clone. Statistical significance was determined using a one-way-ANOVA with post-hoc Dunnett's multiple comparisons test between each set of clonal edited cells and WT.

FIGS. 22A-22C provide extended data for WGD cell lines. FIG. 22A is a propidium iodide flow cytometry ploidy analysis of diploid and WGD HCT116, MCF10A, and RPE1 cell lines. N>=5000 cells per sample. FIG. 22B is shows intensity of BUBR1 at kinetochores in DMSO, KIF18Ai, and Nocodazole treatments between diploid and WGD RPE1 cells from wide-field immunofluorescence images. FIG. 22B (Left) shows the intensity at individual kinetochores. FIG. 22B (Right) shows the summed kinetochore intensity per cell. Violin plots summarize all individual kinetochores analyzed, points represent per-cell intensity measurements, and error bars are mean±SD of per-cell measurements. Statistical significance was determined using an unpaired two-tailed Student's t-test between 2N and 4N KIF18Ai conditions. FIG. 22C shows the intensity of MAD1 at kinetochores in DMSO, KIF18Ai, and Nocodazole treatments between diploid and WGD RPE1 cells from wide-field immunofluorescence images. FIG. 22C (Left) shows the intensity at individual kinetochores. FIG. 22C (Right) shows the summed intensity per cell. Violin plots summarize all individual kinetochores analyzed, points represent per-cell intensity measurements, and error bars are mean±SD of per cell measurements. Statistical significance was determined using an unpaired two-tailed Student's t-test between 2N and 4N KIF18Ai conditions.

FIGS. 23A-23F provide validation of OVCAR-8 CRISPR Cas9 screen hits. FIG. 23A is a schematic of whole-genome CRISPR-Cas9 knockout screen protocol. FIG. 23B is a comparison of OVCAR-8 whole genome CRISPR-Cas9 screens with KIF18Ai at IC50 and IC90 concentrations. Data is annotated with mean±1.5×SD. Genes whose loss is selected against in KIF18Ai with an average FDR <0.1 are highlighted in red. Genes whose loss grants a growth advantage in KIF18Ai with an average FDR <0.1 are highlighted in yellow. FIG. 23C is a Western blot validation of polyclonal OVCAR-8 sgRNA-targeted cell lines for APC4 and UBE2S. FIG. 23D is a Western blot validation of polyclonal OVCAR-8 sgRNA-targeted cell lines for HSET, MAD1, Cyclin B1, and TRIP13. FIG. 23E is a graph from a 5-day MTT endpoint viability assay of polyclonal OVCAR-8 edited cell lines in KIF18Ai. Data are represented as mean±SD. N=3 independent experiments, n=3 technical replicates per experiment. Statistical significance was determined using a one-way-ANOVA with post-hoc Dunnett's multiple comparisons test between edited lines and and WT. FIG. 23F is a graph from a media normalized 5-day MTT endpoint viability assay of polyclonal OVCAR-8 TRIP13 knockout cell lines in KIF18Ai. DMSO normalization was used since TRIP13 knockout cells showed growth rescue in DMN that was not present in other cell lines. Data are represented as mean±SD. N=3 independent experiments, n=3 technical replicates per experiment. Statistical significance was determined using a one-way-ANOVA with post-hoc Dunnett's multiple comparisons test between edited lines and WT.

FIGS. 24A-24E is a validation of APC/C-modulated cell lines. FIG. 24A is a Western blot validation of APC4 and UBE2S knockdown in polyclonal RPE1 diploid and tetraploid cell lines. Arrows indicate the cell line used for unique shRNA experiments. FIG. 24B lists top 10 RNAi co-dependency relationships from DepMap dataset for KIF18A. Outlined dark and light gray points represent sensitive and insensitive cell lines respectively from the panel in FIG. 8C. Bolded table entries are APC/C or SAC genes. FIG. 24C is a Western blot validation of UBE2S overexpression in H2B/α-Tubulin fluorescently tagged HCC1806 cells. FIG. 24D is a quantification of mitotic outcomes from live-cell widefield timelapse microscopy of H2B/α-Tubulin fluorescently tagged HCC1806 WT and UBE2S overexpressing cells colored by mitotic outcome. N=30 cells/condition. FIG. 24E shows metaphase to anaphase duration from live-cell widefield timelapse microscopy of untreated H2B/α-Tubulin fluorescently tagged HCC1806 WT and UBE2S overexpressing cells. Error bars represent mean±SD. Statistical significance was determined using an unpaired two-tailed Student's t-test between WT and UBE2S overexpressing cells.

### DETAILED DESCRIPTION

### Methods of Determining Treatment, Methods of Identifying Responders to Treatment, and Related Methods

The present disclosure provides methods of determining a treatment for a subject with a neoplastic disease (e.g., cancer). In exemplary embodiments, the method comprises assaying a sample obtained from the subject for (a) Spindle Assembly Checkpoint (SAC) activity, (b) ploidy (c) whole genome doubling (WGD), (d) Anaphase Promoting Complex (APC/C) activity, or (e) a combination thereof. In exemplary embodiments, the treatment determined for the subject comprises, consists essentially of, or consists of a KIF18A inhibitor, when the sample is positive for (a) increased SAC signaling or SAC activity, (b) high ploidy, (c) WGD, (d) low APC/C activity, (d) or a combination thereof.

Methods of identifying a subject with a neoplastic disease as sensitive to treatment with a KIF18A inhibitor are provided herein. In exemplary embodiments, the method comprises assaying a sample obtained from the subject for (a) SAC activity, (b) ploidy (c) WGD, (d) APC/C activity, or (e) a combination thereof. In various instances, the subject is identified as sensitive to treatment with a KIF18A inhibitor, when the subject is identified as sensitive to treatment with a KIF18A inhibitor, when the sample is positive for (a) increased SAC signaling or SAC activity, (b) high ploidy, (c) WGD, (d) low APC/C activity, (e) or a combination thereof.

The present disclosure additionally provides a method of identifying a subject with a neoplastic disease as responsive to treatment with a KIF18A inhibitor. In exemplary embodiments, the method comprises determining the sensitivity of the neoplastic disease to treatment with a KIF18A inhibitor. In various instances, the subject is identified as sensitive to treatment with a KIF18A inhibitor, when the subject is identified as sensitive to treatment with a KIF18A inhibitor, when the sample is positive for (a) increased SAC signaling or SAC activity, (b) high ploidy, (c) WGD, (d) low APC/C activity, (e) or a combination thereof.

Methods of maintaining sensitivity of a neoplastic disease to treatment with a KIF18A inhibitor in a subject are provided herein. In exemplary embodiments, the method comprises administering to the subject an agent which lowers APC/C activity. In various instances, the agent inhibits or reduces expression of an APC gene, e.g., ANAPC1, ANAPC2, ANAPC4, ANAPC5, ANAPC7, ANAPC10, ANAPC11, ANAPC13, ANAPC15, ANAPC16, CDC16, CDC23, CDC26, CDC27, UBE2C, UBE2D1, UBE2S. In exemplary embodiments, the method comprises administering to the subject an agent which increases SAC activity. In various instances, the agent promotes activity or expression of BUB1, BUB1B, BUB3, AURKB, CCNB1, MAD1L1, MAD2L1, MAD2L1GP, PPP1CA, PPP1CB, PPP1CC, TRIP13, TPR, USP44, ZNF207, ZW10, or ZWILCH.

### KIF18A Inhibitors

The present disclosure relates to KIF18A inhibitors. The term “KIF18A inhibitor” means any compound useful for modulating KIF18A protein alone or in a bound complex with microtubules (MT) for treating KIF18A-mediated conditions and/or diseases, including neoplastic diseases (e.g., cancer), inflammation, or ciliopathologies. The KIF18A inhibitor compounds disclosed herein have MT-based KIF18A modulatory activity and, in particular, KIF18A inhibitory activity. To this end, the present disclosure also provides the use of these compounds, as well as pharmaceutically acceptable salts thereof, in the preparation and manufacture of a pharmaceutical composition or medicament for therapeutic, prophylactic, acute or chronic treatment of KIF18A mediated diseases and disorders, including without limitation, cancer. Thus, the compounds of the present disclosure are useful in the manufacture of anti-cancer medicaments.

In various aspects, the term “KIF18A inhibitor” means any compound or molecule that targets KIF18A and reduces or inhibits KIF18A activity. KIF18A gene belongs to Kinesin-8 subfamily and is a plus-end-directed motor. KIF18A is believed to influence dynamics at the plus end of kinetochore microtubules to control correct chromosome positioning and spindle tension. Depletion of human KIF18A leads to longer spindles, increased chromosome oscillation at metaphase, and activation of the mitotic spindle assembly checkpoint in Hela cervical cancer cells (MI Mayr et al, Current Biology 17, 488-98, 2007). KIF18A is overexpressed in various types of cancers, including but not limited to colon, breast, lung, pancreas, prostate, bladder, head, neck, cervix, and ovarian cancers. Overexpression of KIF18A dampens sister chromatid oscillation resulting in tight metaphase plates. Inactivation of KIF18A motor function in KIF18A knockout mice or by mutagenic ethylmethanosulfonate (EMS) treatment in KIF18A^{gcd2/gcd2} mice (missense mutation (R308K) in the motor domain) resulting in viable mice with no gross abnormalities in major organs except for clear testis atrophy and sterility (J Stumpff et al Developmental Cell. 2008; 14:252-262; J Stumpff et al Developmental Cell. 2012; 22:1017-1029; XS Liu et al. Genes & Cancer. 2010; 1:26-39; CL Fonseca et al J Cell Biol. 2019; 1-16; A Czechanski et al Developmental Biology. 2015; 402:253-262. O Rath, F Kozielski. Nature Reviews Cancer. 2012; 12:527-539). Normal human and mouse KIF18A-deficient somatic cells were shown to complete cell division with relatively normal mitotic progression but without proper chromosome alignment resulting in daughter cells with a normal karyotype, some defects in exit from mitosis were noted in a subset of normal cells resulting in micronuclei formation on slower proliferation (CL Fonseca et al J Cell Biol. 2019; 1-16). These genetic studies suggest that normal germ and somatic cells have different dependency on requirements for chromosome alignment and indicate that KIF18A may be dispensable in normal euploidy somatic cell division (XS Liu et al Genes & Cancer. 2010; 1:26-39; A Czechanski et al Developmental Biology. 2015; 402:253-262). In normal human tissues, expression of KIF18A is elevated in tissues with actively cycling cells, with highest expression in the testis (GTEx Portal, GTEx Portal, J Lonsdale et al Nature Genetics. 2013:29; 45:580). In various aspects, the KIF18A inhibitor inhibits ATPase activity. For example, the KIF18A inhibitor inhibits MT-ATPase activity and not basal ATPase activity.

The reduction or inhibition provided by the KIF18A inhibitor may not be a 100% or complete inhibition or abrogation or reduction. Rather, there are varying degrees of reduction or inhibition of which one of ordinary skill in the art recognizes as having a potential benefit or therapeutic effect. In this regard, the KIF18A inhibitor may inhibit the KIF18A protein(s) to any amount or level. In exemplary embodiments, the reduction or inhibition provided by the KIF18A inhibitor is at least or about 10% reduction or inhibition (e.g., at least or about 20% reduction or inhibition, at least or about 30% reduction or inhibition, at least or about 40% reduction or inhibition, at least or about 50% reduction or inhibition, at least or about 60% reduction or inhibition, at least or about 70% reduction or inhibition, at least or about 80% reduction or inhibition, at least or about 90% reduction or inhibition, at least or about 95% reduction or inhibition, at least or about 98% reduction or inhibition).

In exemplary instances, the KIF18A inhibitor is described in International Patent Application Publication No. WO2021/211549.

In exemplary instances, the KIF18A inhibitor is 4-(N-(tert-butyl) sulfamoyl)-N-(3-(N-(tert-butyl) sulfamoyl)phenyl)-2-(6-azaspiro[2.5]octan-6-yl)benzamide and/or has the following structure:

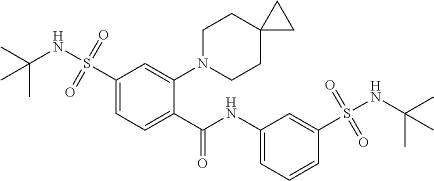 [SMILES: CC(C)(C)NS(=O)(=O)c1cccc(NC(=O)c2ccc(S(=O)(=O)NC(C)(C)C)cc2N2CCC3(CC2)CC3)c1]

or is N-(2-(4,4-Difluoropiperidin-1-yl)-6-methylpyrimidin-4-yl)-4-((2-hydroxyethyl) sulfonamido)-2-(6-azaspiro[2.5]octan-6-yl)benzamide and/or has the following structure:

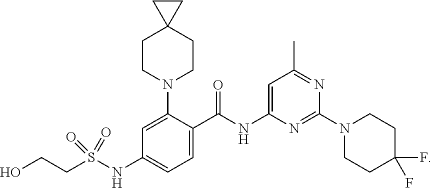 [SMILES: Cc1cc(NC(=O)c2ccc(NS(=O)(=O)CCO)cc2N2CCC3(CC2)CC3)nc(N2CCC(F)(F)CC2)n1]

### Pharmaceutical Compositions, Dosing, And Routes Of Administration

In various aspects, the KIF18A inhibitor is provided as part of a pharmaceutical composition. Accordingly, pharmaceutical compositions including a compound as disclosed herein, together with a pharmaceutically acceptable excipient, such as, for example, a diluent or carrier, are provided by the present disclosure. Compounds and pharmaceutical compositions suitable for use in the present invention include those wherein the compound can be administered in an effective amount to achieve its intended purpose. Administration of the compound described in more detail below.

Suitable pharmaceutical formulations can be determined by the skilled artisan depending on the route of administration and the desired dosage. See, e.g., Remington's Pharmaceutical Sciences, 1435-712 (18th ed., Mack Publishing Co, Easton, Pennsylvania, 1990). Formulations may influence the physical state, stability, rate of in vivo release and rate of in vivo clearance of the administered agents. Depending on the route of administration, a suitable dose may be calculated according to body weight, body surface areas or organ size. Further refinement of the calculations necessary to determine the appropriate treatment dose is routinely made by those of ordinary skill in the art without undue experimentation, especially in light of the dosage information and assays disclosed herein as well as the pharmacokinetic data obtainable through animal or human clinical trials.

The phrases “pharmaceutically acceptable” or “pharmacologically acceptable” refer to molecular entities and compositions that do not produce adverse, allergic, or other untoward reactions when administered to an animal or a human. As used herein, “pharmaceutically acceptable e” includes any and all solvents, dispersion media, coatings, antibacterial and antifungal agents, isotonic and absorption delaying agents and the like. The use of such excipients for pharmaceutically active substances is well known in the art. Except insofar as any conventional media or agent is incompatible with the therapeutic compositions, its use in therapeutic compositions is contemplated. Supplementary active ingredients also can be incorporated into the compositions. In exemplary embodiments, the formulation may comprise corn syrup solids, high-oleic safflower oil, coconut oil, soy oil, L-leucine, calcium phosphate tribasic, L-tyrosine, L-proline, L-lysine acetate, DATEM (an emulsifier), L-glutamine, L-valine, potassium phosphate dibasic, L-isoleucine, L-arginine, L-alanine, glycine, L-asparagine monohydrate, L-serine, potassium citrate, L-threonine, sodium citrate, magnesium chloride, L-histidine, L-methionine, ascorbic acid, calcium carbonate, L-glutamic acid, L-cystine dihydrochloride, L-tryptophan, L-aspartic acid, choline chloride, taurine, m-inositol, ferrous sulfate, ascorbyl palmitate, zinc sulfate, L-carnitine, alpha-tocopheryl acetate, sodium chloride, niacinamide, mixed tocopherols, calcium pantothenate, cupric sulfate, thiamine chloride hydrochloride, vitamin A palmitate, manganese sulfate, riboflavin, pyridoxine hydrochloride, folic acid, beta-carotene, potassium iodide, phylloquinone, biotin, sodium selenate, chromium chloride, sodium molybdate, vitamin D3 and cyanocobalamin.

The compound can be present in a pharmaceutical composition as a pharmaceutically acceptable salt. As used herein, “pharmaceutically acceptable salts” include, for example base addition salts and acid addition salts.

Pharmaceutically acceptable base addition salts may be formed with metals or amines, such as alkali and alkaline earth metals or organic amines. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible. Examples of metals used as cations are sodium, potassium, magnesium, ammonium, calcium, or ferric, and the like. Examples of suitable amines include isopropylamine, trimethylamine, histidine, N,N′-dibenzylethylenediamine, chloroprocaine, choline, diethanolamine, dicyclohexylamine, ethylenediamine, N-methylglucamine, and procaine.

Pharmaceutically acceptable acid addition salts include inorganic or organic acid salts. Examples of suitable acid salts include the hydrochlorides, formates, acetates, citrates, salicylates, nitrates, phosphates. Other suitable pharmaceutically acceptable salts are well known to those skilled in the art and include, for example, formic, acetic, citric, oxalic, tartaric, or mandelic acids, hydrochloric acid, hydrobromic acid, sulfuric acid or phosphoric acid; with organic carboxylic, sulfonic, sulfo or phospho acids or N-substituted sulfamic acids, for example acetic acid, trifluoroacetic acid (TFA), propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha amino acids involved in the synthesis of proteins in nature, for example glutamic acid or aspartic acid, and also with phenylacetic acid, methanesulfonic acid (mesylate), toluenesulfonic acids (tosylate), ethanesulfonic acid, 2-hydroxyethanesulfonic acid, ethane 1,2-disulfonic acid, benzenesulfonic acid (besylate), 4-methylbenzenesulfonic acid, naphthalene 2-sulfonic acid, naphthalene 1,5-disulfonic acid, 2- or 3-phosphoglycerate, glucose 6-phosphate, N-cyclohexylsulfamic acid (with the formation of cyclamates), or with other acid organic compounds, such as ascorbic acid.

Pharmaceutical compositions containing the compounds disclosed herein can be manufactured in a conventional manner, e.g., by conventional mixing, dissolving, granulating, dragee-making, levigating, emulsifying, encapsulating, entrapping, or lyophilizing processes. Proper formulation is dependent upon the route of administration chosen.

For oral administration, suitable compositions can be formulated readily by combining a compound disclosed herein with pharmaceutically acceptable excipients such as carriers well known in the art. Such excipients and carriers enable the present compounds to be formulated as tablets, pills, dragees, capsules, liquids, gels, syrups, slurries, suspensions and the like, for oral ingestion by a patient to be treated. Pharmaceutical preparations for oral use can be obtained by adding a compound as disclosed herein with a solid excipient, optionally grinding a resulting mixture, and processing the mixture of granules, after adding suitable auxiliaries, if desired, to obtain tablets or dragee cores. Suitable excipients include, for example, fillers and cellulose preparations. If desired, disintegrating agents can be added. Pharmaceutically acceptable ingredients are well known for the various types of formulation and may be for example binders (e.g., natural or synthetic polymers), lubricants, surfactants, sweetening and flavoring agents, coating materials, preservatives, dyes, thickeners, adjuvants, antimicrobial agents, antioxidants and carriers for the various formulation types.

When a therapeutically effective amount of a compound disclosed herein is administered orally, the composition typically is in the form of a solid (e.g., tablet, capsule, pill, powder, or troche) or a liquid formulation (e.g., aqueous suspension, solution, elixir, or syrup).

When administered in tablet form, the composition can additionally contain a functional solid and/or solid carrier, such as a gelatin or an adjuvant. The tablet, capsule, and powder can contain about 1 to about 95% compound, and preferably from about 15 to about 90% compound.

When administered in liquid or suspension form, a functional liquid and/or a liquid carrier such as water, petroleum, or oils of animal or plant origin can be added. The liquid form of the composition can further contain physiological saline solution, sugar alcohol solutions, dextrose or other saccharide solutions, or glycols. When administered in liquid or suspension form, the composition can contain about 0.5 to about 90% by weight of a compound disclosed herein, and preferably about 1 to about 50% of a compound disclosed herein. In one embodiment contemplated, the liquid carrier is non-aqueous or substantially non-aqueous. For administration in liquid form, the composition may be supplied as a rapidly-dissolving solid formulation for dissolution or suspension immediately prior to administration.

When a therapeutically effective amount of a compound disclosed herein is administered by intravenous, cutaneous, or subcutaneous injection, the composition is in the form of a pyrogen-free, parenterally acceptable aqueous solution. The preparation of such parenterally acceptable solutions, having due regard to pH, isotonicity, stability, and the like, is within the skill in the art. A preferred composition for intravenous, cutaneous, or subcutaneous injection typically contains, in addition to a compound disclosed herein, an isotonic vehicle. Such compositions may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations can optionally contain a preservative to prevent the growth of microorganisms.

Injectable compositions can include sterile aqueous solutions, suspensions, or dispersions and sterile powders for the extemporaneous preparation of sterile injectable solutions, suspensions, or dispersions. In all embodiments the form must be sterile and must be fluid to the extent that easy syringability exists. It must be stable under the conditions of manufacture and storage and must resist the contaminating action of microorganisms, such as bacteria and fungi, by optional inclusion of a preservative. The carrier can be a solvent or dispersion medium containing, for example, water, ethanol, polyol (e.g., glycerol, propylene glycol, and liquid polyethylene glycol, and the like), suitable mixtures thereof, and vegetable oils. In one embodiment contemplated, the carrier is non-aqueous or substantially non-aqueous. The proper fluidity can be maintained, for example, by the use of a coating, such as lecithin, by the maintenance of the required particle size of the compound in the embodiment of dispersion and by the use of surfactants. The prevention of the action of microorganisms can be brought about by various antibacterial and antifungal agents, for example, parabens, chlorobutanol, phenol, sorbic acid, thimerosal, and the like. In many embodiments, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the embodiment of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Slow release or sustained release formulations may also be prepared in order to achieve a controlled release of the active compound in contact with the body fluids in the GI tract, and to provide a substantially constant and effective level of the active compound in the blood plasma. For example, release can be controlled by one or more of dissolution, diffusion, and ion-exchange. In addition, the slow release approach may enhance absorption via saturable or limiting pathways within the GI tract. For example, the compound may be embedded for this purpose in a polymer matrix of a biological degradable polymer, a water-soluble polymer or a mixture of both, and optionally suitable surfactants. Embedding can mean in this context the incorporation of micro-particles in a matrix of polymers. Controlled release formulations are also obtained through encapsulation of dispersed micro-particles or emulsified micro-droplets via known dispersion or emulsion coating technologies.

For administration by inhalation, compounds of the present invention are conveniently delivered in the form of an aerosol spray presentation from pressurized packs or a nebulizer, with the use of a suitable propellant. In the embodiment of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

The compounds disclosed herein can be formulated for parenteral administration by injection (e.g., by bolus injection or continuous infusion). Formulations for injection can be presented in unit dosage form (e.g., in ampules or in multidose containers), with an added preservative. The compositions can take such forms as suspensions, solutions, or emulsions in oily or aqueous vehicles, and can contain formulatory agents such as suspending, stabilizing, and/or dispersing agents.

Pharmaceutical formulations for parenteral administration include aqueous solutions of the compounds in water-soluble form. Additionally, suspensions of the compounds can be prepared as appropriate oily injection suspensions. Suitable lipophilic solvents or vehicles include fatty oils or synthetic fatty acid esters. Aqueous injection suspensions can contain substances which increase the viscosity of the suspension. Optionally, the suspension also can contain suitable stabilizers or agents that increase the solubility of the compounds and allow for the preparation of highly concentrated solutions. Alternatively, a present composition can be in powder form for constitution with a suitable vehicle (e.g., sterile pyrogen-free water) before use.

Compounds disclosed herein also can be formulated in rectal compositions, such as suppositories or retention enemas (e.g., containing conventional suppository bases). In addition to the formulations described previously, the compounds also can be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In particular, a compound disclosed herein can be administered orally, buccally, or sublingually in the form of tablets containing excipients, such as starch or lactose, or in capsules or ovules, either alone or in admixture with excipients, or in the form of elixirs or suspensions containing flavoring or coloring agents. Such liquid preparations can be prepared with pharmaceutically acceptable additives, such as suspending agents. A compound also can be injected parenterally, for example, intravenously, intramuscularly, subcutaneously, or intracoronarily. For parenteral administration, the compound is best used in the form of a sterile aqueous solution which can contain other substances, for example, salts, or sugar alcohols, such as mannitol, or glucose, to make the solution isotonic with blood.

For veterinary use, a compound disclosed herein is administered as a suitably acceptable formulation in accordance with normal veterinary practice. The veterinarian can readily determine the dosing regimen and route of administration that is most appropriate for a particular animal.

In some embodiments, all the necessary components for the treatment of KIF18A-related disorder using a compound as disclosed herein either alone or in combination with another agent or intervention traditionally used for the treatment of such disease may be packaged into a kit. Specifically, the present invention provides a kit for use in the therapeutic intervention of the disease comprising a packaged set of medicaments that include the compound disclosed herein as well as buffers and other components for preparing deliverable forms of said medicaments, and/or devices for delivering such medicaments, and/or any agents that are used in combination therapy with the compound disclosed herein, and/or instructions for the treatment of the disease packaged with the medicaments. The instructions may be fixed in any tangible medium, such as printed paper, or a computer readable magnetic or optical medium, or instructions to reference a remote computer data source such as a world wide web page accessible via the internet.

A “therapeutically effective amount” means an amount effective to treat or to prevent development of, or to alleviate the existing symptoms of, the subject being treated. Determination of the effective amounts is well within the capability of those skilled in the art, especially in light of the detailed disclosure provided herein. Generally, a “therapeutically effective dose” refers to that amount of the compound that results in achieving the desired effect. For example, in one preferred embodiment, a therapeutically effective amount of a compound disclosed herein decreases KIF18A activity by at least 5%, compared to control, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, or at least 90%.

The amount of compound administered can be dependent on the subject being treated, on the subject's age, health, sex, and weight, the kind of concurrent treatment (if any), severity of the affliction, the nature of the effect desired, the manner and frequency of treatment, and the judgment of the prescribing physician. The frequency of dosing also can be dependent on pharmacodynamic effects on arterial oxygen pressures. While individual needs vary, determination of optimal ranges of effective amounts of the compound is within the skill of the art. Such doses may be administered in a single dose or it may be divided into multiple doses.

### APC/C Activity, SAC Activity, and Related Genes

In various aspects, the methods of the present disclosure comprise assaying for APC/C activity. The APC/C is also known as the cyclosome and is a protein complex comprising 11-13 protein subunits. The APC/C is further described in Castro et al., Oncogene 24:314-325 (2005). In various aspects, assaying for APC/C activity comprises assaying the sample for phosphorylation of the APC/C or a subunit thereof, e.g., Apc1, Apc3/Cdc27, Apc6/Cdc16, Apc7, or Apc8/Cdc23. In exemplary aspects, assaying for APC/C activity comprises assaying the sample for expression levels of RNA or protein encoded by one or more genes encoding the APC/C. In exemplary aspects, the assaying step comprises assaying the sample for expression levels of RNA or protein encoded by one or more of the following genes: ANAPC1, ANAPC2, ANAPC4, ANAPC5, ANAPC7, ANAPC10, ANAPC11, ANAPC13, ANAPC15, ANAPC16, CDC16, CDC23, CDC26, CDC27, UBE2C, UBE2D1, and UBE2S.

In various aspects, the methods of the present disclosure comprise assaying for SAC activity. The SAC is further described in Musacchio and Salmon, Nat Rev Molec Cell Biol 8:379-393 (2007). The SAC targets a protein CDC20 which is a co-factor of the APC/C. The SAC negatively regulates the ability of CDC20 to activate APC/C. In various aspects, assaying for SAC activity comprises assaying the sample for SAC signaling. In exemplary aspects, assaying for SAC activity comprises assaying the sample for expression levels of RNA or protein encoded by one or more genes encoding SAC. In exemplary instances, the assaying step comprises assaying the sample for expression levels of RNA or protein encoded by one or more of the following genes: BUB1, BUB1B, BUB3, AURKB, CCNB1, MAD1L1, MAD2L1, MAD2L1GP, PPP1CA, PPP1CB, PPP1CC, TRIP13, TPR, USP44, ZNF207, ZW10, and ZWILCH.

In various aspects, the assaying step comprises measuring ploidy and/or WGD via chromosome counting (via e.g., karyotyping, parallel sequencing, comparative genomic hybridization (CGH), microarrays) high throughput sequencing (HTS), or flow cytometry. Suitable methods of measuring ploidy and/or WGD are known in the art. See, e.g., Viruel et al., Frontiers in Plant Science 10 (2019); doi: 10.3389/fpls.2019.00937, Carter et al., Nature Biotech 30 (5): 413-426 (2012). In exemplary aspects, the ploidy is high or is greater than 2.0 or 2.1. In exemplary aspects, the WGD is present and has a ploidy greater than 2.0 or 2.1.

The assaying allows for the sample to be identified as “positive” or “negative” for (a) increased SAC signaling or SAC activity, (b) high ploidy, (c) WGD, (d) low APC/C activity, (e) or a combination thereof. As used herein, the term “positive” in the context of a sample means that (a) increased SAC signaling or SAC activity, (b) high ploidy, (c) WGD, (d) low APC/C activity, (e) or a combination thereof, is/are present in the sample. As used herein, the term “negative” in the context of a sample means that (a) increased SAC signaling or SAC activity, (b) high ploidy, (c) WGD, (d) low APC/C activity, (e) or a combination thereof, is/are absent from the sample. In various instances, the sample is positive for increased SAC activity or SAC signaling. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level. In various aspects, the increased SAC activity or SAC signaling refers to a level of SAC activity or SAC signaling which is increased by at least or about 50%, at least or about 55%, at least or about 60%, at least or about 65%, at least or about 70%, at least or about 75%, at least or about 80%, at least or about 85%, at least or about 90%, or at least or about 95%, relative to a control level. In various instances, the increased SAC activity or SAC signaling refers to about greater than or about 120% (e.g., 125%, 130%, 135%, 140%, 145%, 150%, 200%, 250%, 300%, or more) of the SAC activity observed in control samples. Optionally, the control samples are samples comprising normal levels of SAC activity. For example, the control samples are obtained from normal subjects known to not have cancer. In various instances, the sample is positive for low APC/C activity or APC/C signaling. As used herein, the term “low” means “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level. In various aspects, the low APC/C activity refers to a level of APC/C activity which is about 50% or less (e.g., 45%, 40%, 35%, 30%, 25%, 20%, 15%, 10%, 5% or less) of the APC/C activity observed in control samples. Optionally, the control samples are samples comprising normal levels of APC/C activity. For example, the control samples are obtained from normal subjects known to not have cancer.

The wild-type genes, as well as the RNA and proteins encoded by the aforementioned genes, are known in the art. Exemplary sequences of each are available at the website for the National Center for Biotechnology Information (NCBI) and herein as set forth below.

TABLE A

| Gene | Gene | Example RefSeq | SEQ |
|---|---|---|---|
| Name | ID | Accession No. | ID NO: |
| ANAPC1 | 64682 | NP_073153.1 | 1 |
| ANAPC10 | 10393 | NP_001243635.1 | 2 |
| ANAPC11 | 10393 | NP_001002248.1 | 3 |
| ANAPC13 | 25847 | NP_056206.1 | 4 |
| ANAPC15 | 25906 | NP_054761.1 | 5 |
| ANAPC16 | 119504 | NP_775744.1 | 6 |
| ANAPC2 | 29882 | NP_037498 | 7 |
| ANAPC4 | 29945 | NP_037499.2 | 8 |
| ANAPC5 | 51433 | NP_057321.2 | 9 |
| ANAPC7 | 51434 | NP_057322.3 | 10 |
| CDC16 | 8881 | NP_001072113.1 | 11 |
| CDC23 | 8697 | NP_004652.2 | 12 |
| CDC26 | 246184 | NP_644815.1 | 13 |
| CDC27 | 996 | NP_001247.3 | 14 |
| UBE2C | 11065 | NP_008950.1 | 15 |
| UBE2D1 | 7321 | NP_003329.1 | 16 |
| UBE2S | 27338 | NP_055316.2 | 17 |
| BUB1 | 699 | NP_004327.1 | 18 |
| BUB1B | 701 | NP_001202.5 | 19 |
| BUB3 | 9184 | NP_004716.1 | 20 |
| AURKB | 9212 | NP_004208.2 | 21 |
| CCNB1 | 891 | NP_114172.1 | 22 |
| MAD1L1 | 8379 | NP_001013858.1 | 23 |
| MAD2L1 | 4085 | NP_002349.1 | 24 |
| MAD2L1BP | 9587 | NP_055443.1 | 25 |
| PPP1CA | 5499 | NP_002699.1 | 26 |
| PPP1CB | 5500 | NP_002700.1 | 27 |
| PPP1CC | 5501 | NP_002701.1 | 28 |
| TRIP13 | 9319 | NP_004228.1 | 29 |
| TPR | 7175 | NP_003283.2 | 30 |
| USP44 | 84010 | NP_115523.2 | 31 |
| ZNF207 | 7756 | NP_001091977.1 | 32 |
| ZW10 | 9183 | NP_004715.1 | 33 |
| ZWILCH | 55055 | NP_060445.3 | 34 |

Gene ID corresponds to the Gene database of the National Center for Biotechnology Information (NCBI) and RefSeq Accession No. corresponds to the Protein database of NCBI.

In exemplary aspects, the assaying step comprises assaying the sample for inactivation of one or more of the aforementioned genes. In various embodiments of the methods of the present disclosure, the methods comprise assaying a sample for an inactivated gene. As used herein, the term “inactivated” in the context of a gene refers to a reduction or loss of function of the gene or gene product encoded by the gene. The inactivation of a gene may be caused by one or more known mechanisms. For example, the inactivation of the gene may be caused by a variation in (including, e.g., a loss of) DNA sequence, RNA sequence or protein sequence, relative to the corresponding wild-type gene, RNA, or protein or may be caused by an epigenetic variation that does not involve any alterations in the DNA sequence of the gene.

In exemplary aspects, the assaying step comprises assaying the sample for variation of one or more of the aforementioned genes. In various aspects, the assaying step comprises detecting the presence of the variation or anomaly in a gene or a gene product encoded by the gene, which variation or anomaly is relative to the corresponding wild-type gene or gene product, and which presence of the variation leads to or is associated with a silencing of the gene, a reduction or loss of expression of the gene or gene product encoded by the gene, a reduction or loss of function of the gene or gene product encoded by the gene, or a combination thereof. In various instances, the gene product is an RNA transcript or a protein. In various instances, the variation leads to at least a reduction or loss of function of the gene or gene product encoded by the gene. The variation in the gene may be present anywhere in the gene, e.g., within an intron or exon, within a 5′-untranslated region (5′-UTR), or a 3′-untranslated region (3′-UTR). The variation may be present within or at any part of the transcript (e.g., RNA transcript, primary transcript, pre-mRNA, mRNA) encoded by the gene, or may be present within or at any part of the protein encoded by the gene. In various aspects, the variation is a difference in DNA sequence, RNA sequence or protein sequence, relative to the corresponding wild-type gene, RNA, or protein. In various aspects, the sample is assayed for the inactivated gene by analyzing the nucleotide sequence of the gene, analyzing the nucleotide sequence of an RNA encoded by the gene, or analyzing the amino acid sequence of the protein encoded by the gene and comparing the sequence of gene of the sample to the corresponding wild-type human sequence of the gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein. In exemplary aspects, the variation comprises a deletion, insertion, or substitution of one or more nucleotides in the DNA sequence or RNA sequence, a deletion, insertion, or substitution of one or more amino acids in the protein sequence, relative to the corresponding wild-type gene, RNA, or protein that may result in a gene copy number gain or amplification of the DNA, RNA, or protein. In various aspects, the assaying comprises detecting the presence of a gene mutation in the gene. In various aspects, the assaying comprises detecting the presence of a gene mutation in the gene or loss of nucleotides in the gene. In exemplary instances, the gene mutation is a missense mutation, nonsense mutation, insertion, deletion, duplication, frameshift mutation, truncation, or a repeat expansion. In various instances, the variation is epigenetic and does not involve any alterations in the DNA sequence of the gene. In exemplary aspects, the inactivated gene is epigenetically silenced and optionally involves a covalent modification of the DNA or histone proteins. The covalent modification of the DNA may be, for example, a cytosine methylation or hydroxymethylation. The covalent modification of the histone protein may be, for example, a lysine acetylation, lysine or arginine methylation, serine or threonine phosphorylation, or lysine ubiquitination or sumoylation. Mechanisms of gene silencing can occur during transcription or translation. Exemplary mechanisms of gene silencing include but are not limited to DNA methylation, histone modification, and RNA interference (RNAi). In various aspects, the inactivated gene is an epigenetically silenced gene having an epigenetically silenced promoter. Suitable techniques to assay for epigenetic silencing include but are not limited to chromatin immunoprecipitation (ChIP-on chip, ChIP-Seq) fluorescent in situ hybridization (FISH), methylation-sensitive restriction enzymes, DNA adenine methyltransferase identification (DamID) and bisulfite sequencing. See, e.g., Verma et. al., Cancer Epidemiology, Biomarkers, and Prevention 23:223-233 (2014).

In exemplary aspects, the assaying step comprises assaying the sample for amplification of one or more of the aforementioned genes. In various embodiments of the methods of the present disclosure, the methods comprise assaying a sample for a gene amplification, or an increase in the number of copies of a gene, e.g., a gene copy number gain of the gene. In various instances, the sample is assayed for the gain or amplified gene by DNA- or RNA-based techniques (gene expression analysis [comparative genomic hybridization, RNA-based hybridization], NGS, PCR, or Southern blot) or by molecular cytogenetic techniques (FISH2 with gene-specific probes, CISH (chromogenic in situ hybridization). In various aspects, competitive or quantitative PCR, genomic hybridization to cDNA microarrays, hybridization and quantification of gene probes to RNA are carried out to detect the gene amplification or gene copy number gain. See., e.g., Harlow and Stewart, Genome Res 3:163-168 (1993); Heiskanen et. al., Cancer Res 60 (4): 799-802 (2000). Next Generation Sequencing (NGS) may also be employed as a method by which to detect a gene copy number gain or loss or a gene amplification whereby genetic areas are sequenced and sequencing reads are compared to other genes to deduce gain or loss of the gene of interest.

In exemplary instances, the assaying step comprises a cytogenetics method and/or molecular method for detecting the presence of an inactivated or amplified gene or gene copy number gain. In exemplary aspects, the assaying step comprises direct DNA sequencing, DNA hybridization and/or restriction enzyme digestion. Optionally, the cytogenetics method comprises karyotyping, fluorescence in situ hybridization (FISH), comparative genomic hybridization (CGH), or a combination thereof. In various instances, the molecular method comprises restriction fragment length polymorphism (RFLP), amplification refractory mutation system (ARMS), polymerase chain reaction (PCR), multiplex ligation dependent probe amplification (MLPA), denaturing gradient gel electrophoresis (DGGE), single strand conformational polymorphism (SSCP), heteroduplex analysis, chemical cleavage of mismatch (CCM), protein truncation test (PTT), oligonucleotide ligation assay (OLA), or a combination thereof. Optionally, the PCR is a multiplex PCR, nested PCR, RT-PCR, or real time quantitative PCR. In various aspects, the assaying step comprises ARMS, FISH, IHC, or NGS. Such techniques are described in Su et al., J Experimental Clin Cancer Research 36:121 (2017) and He et al., Blood 127 (24): 3004-3014 (2016). In various instances, the assaying step comprises whole-exome sequencing or whole genome sequencing. In exemplary aspects, the assaying comprises a liquid biopsy. Liquid biopsies are described in detail in the art. See, e.g., Poulet et al., Acta Cytol 63 (6): 449-455 (2019), Chen and Zhao, Hum Genomics 13 (1): 34 (2019).

In various aspects, the gene copy number gain or amplification leads to overexpressed or increased levels of the gene products (e.g., RNA and/or protein) encoded by the gene. Methods of detecting increased levels in RNA and/or protein are known in the art.

In various instances, the methods of the present disclosure comprise measuring a level of expression of a gene, via RNA transcripts, e.g., a messenger RNA (mRNA), or a protein, in a sample (e.g., a sample comprising tissue or blood) obtained from a subject. In exemplary aspects of the presently disclosed methods, the method comprises measuring the level of expression of an APC/C gene or SAC gene, or any gene product encoded by the gene, or any combination thereof. Suitable methods of determining expression levels of nucleic acids (e.g., genes, RNA, mRNA) are known in the art and include but not limited to, quantitative polymerase chain reaction (qPCR) (e.g., quantitative real-time PCR (qRT-PCR)), RNAseq, Nanostring, and Northern blotting. Techniques for measuring gene expression also include, for example, gene expression assays with or without the use of gene chips or gene expression microarrays are described in Onken et. al., J Molec Diag 12 (4): 461-468 (2010); and Kirby et. al., Adv Clin Chem 44:247-292 (2007). Affymetrix gene chips and RNA chips and gene expression assay kits (e.g., Applied Biosystems™ TaqMan® Gene Expression Assays) are also commercially available from companies, such as ThermoFisher Scientific (Waltham, MA), and Nanostring (Geiss et. al., Nature Biotechnology 26:317-325 (2008)). Suitable methods of determining expression levels of proteins are known in the art and include immunoassays (e.g., Western blotting, an enzyme-linked immunosorbent assay (ELISA), a radioimmunoassay (RIA), and immunohistochemical assay) or bead-based multiplex assays, e.g., those described in Djoba Siawaya J F, Roberts T, Babb C, Black G, Golakai H J, Stanley K, et al. (2008) An Evaluation of Commercial Fluorescent Bead-Based Luminex Cytokine Assays. PLOS ONE 3 (7): e2535. Proteomic analysis which is the systematic identification and quantification of proteins of a particular biological system are known. Mass spectrometry is typically the technique used for this purpose.

In exemplary aspects, the method comprises measuring the level of a complementary DNA (cDNA) based on the RNA encoded by said gene. Briefly, the method comprises extracting or isolating RNA from the sample (e.g., from the tumor cell(s) of the sample) and synthesizing cDNA based on RNA isolated from the sample. Alternatively or additionally, in some aspects, measuring the expression level comprises isolating RNA from the sample, producing complementary DNA (cDNA) from the RNA, amplifying the cDNA and hybridizing the cDNA to a gene expression microarray. Accordingly, in some aspects, measuring the expression level comprises isolating RNA from the sample and quantifying the RNA by RNA-Seq. In alternative or additional aspects, the level of expression is determined via an immunohistochemical assay.

Once the expression level of the gene, or the gene product thereof, is measured from the sample obtained from the subject, the measured expression level may be compared to a reference level, normalized to a housekeeping gene, mathematically transformed. In exemplary instances, the measured expression level of the gene, or the gene product thereof, is centered and scaled. Suitable techniques of centering and scaling biological data are known in the art. See, e.g., van den Berg et. al., BMC Genomics 7:142 (2006).

In exemplary embodiments, the methods comprise measuring additional genes, RNA, and/or proteins not listed in Table A. In exemplary embodiments, the methods comprise measuring the expression level of at least one additional gene, RNA, or protein. In exemplary instances, the methods comprise measuring the expression level of at least 2, 3, 4, 5 or more additional genes, at least 2, 3, 4, 5 or more additional RNA, and/or at least 2, 3, 4, 5 or more additional proteins in the sample. In exemplary instances, the methods comprise measuring the expression level of at least 10, 15, 20 or more additional genes, at least 10, 15, 20 or more additional RNA, and/or at least 10, 15, 20 or more additional proteins in the sample. In exemplary instances, the methods comprise measuring the expression level of at least 50, 100, 200 or more additional genes, at least 50, 100, 200 or more additional RNA, and/or at least 50, 100, 200 or more additional proteins in the sample. In exemplary instances, the methods comprise measuring the expression level of a plurality of different genes, a plurality of RNA, and/or a plurality of proteins, in addition to one or more listed in Table A. In various instances, the additional proteins include a protein encoded by a gene listed in Table B and/or Table C.

TABLE B

| Gene | Gene ID | Example RefSeq Accession No. |
|---|---|---|
| SUPT20H | 55578 | NP_001014308 |
| CCDC101 | 112869 | NP_612423.1 |
| C4orf45 | 152940 | NP_689756.2 |
| GLTSCR1L | 428568 | XP_040553328.1 |
| TMEM236 | 653567 | NP_001092314.1 |
| COX4I2 | 84701 | NP_115998.2 |
| LARP7 | 51574 | NP_001253968.2 |
| KDM2B | 84678 | NP_001005366.1 |
| SP5 | 389058 | NP_001003845.1 |
| TAF6L | 10629 | NP_006464.1 |
| LMBRD1 | 55788 | NP_001350651.1 |
| MYLK2 | 85366 | NP_149109.1 |
| TCEB3 (ELOA) | 6924 | NP_003189.3 |
| ETFB | 2109 | NP_001014763.1 |
| TSC22D2 | 9819 | NP_001290193.1 |
| HTR3D | 200909 | NP_001138615.1 |

TABLE C

| | Gene ID | Example RefSeq Accession No. |
|---|---|---|
| KATNA1 | 11104 | NP_001191005.1 |
| PRPF38A | 84950 | NP_116253.2 |
| PRPF39 | 55015 | NP_060392.3 |
| TBL1XR1 | 79718 | NP_001308122.1 |
| SNRNP40 | 9410 | NP_004805.2 |
| WDR62 | 284403 | NP_001077430.1 |
| DSN1 | 79980 | NP_001138787.1 |
| KIF22 | 3835 | NP_001243198.1 |
| INTS10 | 55174 | NP_001340434.1 |
| INO80 | 54617 | NP_060023.1 |
| LOC102724862 | 102724862 | NP_001278392.1 |
| (TBC1D3I) | | |
| PAFAH1B1 | 5048 | NP_000421.1 |
| LCMT1 | 51451 | NP_001027563.1 |
| DCTN2 | 10540 | NP_001248341.1 |
| PPME1 | 51400 | NP_001258522.1 |
| PPP4C | 5531 | NP_001290432.1 |

### Responsiveness, Sensitivity and Resistance

The present disclosure relates to responsiveness, sensitivity and/or resistance to a drug, e.g., KIF18A inhibitor. The present disclosure provides a method of identifying a subject with a neoplastic disease as sensitive or responsive to treatment with a KIF18A inhibitor is provided herein.

As used herein “sensitivity” refers to the way a neoplastic disease (e.g., cancer, tumor) reacts to a drug/compound, e.g., a KIF18A inhibitor). In exemplary aspects, “sensitivity” means “responsive to treatment” and the concepts of “sensitivity” and “responsiveness” are positively associated in that a neoplastic disease (e.g., tumor or cancer cell) that is responsive to a drug/compound treatment is said to be sensitive to that drug. “Sensitivity” in exemplary instances is defined according to Pelikan, Edward, Glossary of Terms and Symbols used in Pharmacology (Pharmacology and Experimental Therapeutics Department Glossary at Boston University School of Medicine), as the ability of a population, an individual or a tissue, relative to the abilities of others, to respond in a qualitatively normal fashion to a particular drug dose. The smaller the dose required producing an effect, the more sensitive is the responding system. “Sensitivity” may be measured or described quantitatively in terms of the point of intersection of a dose-effect curve with the axis of abscissal values or a line parallel to it; such a point corresponds to the dose just required to produce a given degree of effect. In analogy to this, the “sensitivity” of a measuring system is defined as the lowest input (smallest dose) required producing a given degree of output (effect). In exemplary aspects, “sensitivity” is opposite to “resistance” and the concept of “resistance” is negatively associated with “sensitivity”. For example, a tumor that is resistant to a drug treatment is either not sensitive nor responsive to that drug or was initially sensitive to the drug and is no longer sensitive upon acquiring resistance; that drug is not an effective treatment for that tumor or cancer cell.

The term “responsiveness” as used herein refers to the extent of a therapeutic response or responsiveness of a cancer cell or tumor to a drug/compound (e.g., a KIF18A inhibitor) or other treatment (e.g., radiation therapy) as per Response Evaluation Criteria in Solid Tumors (RECIST) or other like criteria. RECIST is a set of criteria to evaluate the progression, stabilization or responsiveness of tumors and/or cancer cells jointly created by the National Cancer Institute of the United States, the National Cancer Institute of Canada Clinical Trials Group and the European Organisation for Research and Treatment of Cancer. According to RECIST, certain tumors are measured in the beginning of an evaluation (e.g., a clinical trial), in order to provide a baseline for comparison after treatment with a drug. The response assessment and evaluation criteria for tumors are published in Eisenhauer et. al., Eur J Cancer 45:228-247 (2009) and Litière et. al., Journal of Clinical Oncology 37 (13): 1102-1110 (2019) DOI: 10.1200/JCO.18.01100. Briefly, Section 4.3 of Eisenhauer et. al., 2009, supra, teaches response criteria to be used to determine objective tumor response for target lesions, as follows:

| Response | |
|---|---|
| Type | Signifies that: |
| Complete | Disappearance of all target lesions. Any pathological |
| Response (CR) | lymph nodes (whether target or non-target) must have |
| | reduction in short axis to <10 mm. |
| Partial | At least a 30% decrease in the sum of diameters of target |
| Response (PR) | lesions, taking as reference the baseline sum diameters. |
| Stable | Neither sufficient shrinkage to qualify for PR nor |
| Disease (SD) | sufficient increase to qualify for PD, taking as reference |
| | the smallest sum diameters while on study |
| Progressive | At least a 20% increase in the sum of diameters of target |
| Disease (PD) | lesions, taking as reference the smallest sum on study |
| | (this includes the baseline sum if that is the smallest on |
| | study). In addition to the relative increase of 20%, the |
| | sum must also demonstrate an absolute increase of at |
| | least 5 mm. (Note: the appearance of one or more new |
| | lesions is also considered progression). |

In ideal cases, a drug or other treatment results in CR or PR as best over-all response with durable duration of response (DOR). Responses of SD with short DOR or PD in some aspects are used to show that a drug is not an effective treatment for cancer or that a tumor has stopped responding to treatment.

In exemplary aspects, responsiveness accounts for or is based on clinical benefit rate (CBR) which is defined as the proportion of patients in whom the best overall response is determined as complete response (CR), partial response (PR) or stable disease (SD)>16 weeks and 24 weeks. Optionally, the CBR relates to proportion of patients in whom the best overall response is determined as complete response (CR), partial response (PR) or stable disease (SD)>16 weeks and 24 weeks wherein the patients have refractory or relapsed breast cancer or ovarian cancer.

As recognized by one of ordinary skill in the art, such a tumor or cancer cell is understood as one that has lost sensitivity to treatment and/or one that has become resistant to treatment.

Provided herein are methods of identifying a subject with a neoplastic disease as sensitive or responsive to treatment with a KIF18A inhibitor. In exemplary embodiments, the method comprises assaying a sample obtained from the subject for (a) SAC activity, (b) ploidy (c) WGD, (d) APC/C activity, or (e) a combination thereof, wherein the subject is identified as sensitive to treatment with a KIF18A inhibitor, when the sample is positive for (a) increased SAC signaling or SAC activity, (b) high ploidy, (c) WGD, (d) low APC/C activity, (e) or a combination thereof.

In various instances of the presently disclosed methods, the sensitivity to a KIF18A inhibitor is determined by assaying a sample obtained from the subject for (a) SAC activity, (b) ploidy (c) WGD, (d) APC/C activity, or (e) a combination thereof.

Methods of maintaining sensitivity of a neoplastic disease to treatment with a KIF18A inhibitor in a subject are provided herein. In exemplary embodiments, the method comprises administering to the subject an agent which lowers APC/C activity or increases SAC signaling or activity. In various aspects, at least 50% of the sensitivity to the treatment is maintained. Optionally, at least or about a 50% increase, at least or about a 60% increase, at least or about a 70% increase, at least or about a 80% increase, at least or about a 90% increase, at least or about a 95% increase, or at least or about a 98% increase, at least or about a 100% increase) of the sensitivity to the treatment is maintained.

### Additional Steps

With regard to the methods of the invention, the methods may include additional steps. For example, the method may include repeating one or more of the recited step(s) of the method. Accordingly, in exemplary aspects, the method comprises assaying a second sample obtained from the subject for (a) SAC activity, (b) ploidy (c) whole genome doubling (WGD), (d) Anaphase Promoting Complex (APC/C) activity, or (e) a combination thereof, wherein the second sample is obtained from the subject at a different time point, relative to the time at which the first sample was obtained from the subject. In exemplary aspects, the method comprises assaying a sample obtained from the subject every month, every 2 months, every 3 months, every 4 months, or every 6 to 12 months, wherein the assaying is based on a different sample obtained from the same subject.

In exemplary aspects, the presently disclosed method further comprises obtaining a sample from the subject. In various aspects, a sample is obtained by blood draw, apheresis, leukapheresis, biopsy or by collection of urine.

In exemplary aspects, the method further comprises administering a KIF18A inhibitor once the need therefor has been determined. Methods of administering a KIF18A inhibitor to a subject may be the same as or similar to any of the presently disclosed methods of administering a pharmaceutical combination.

In various aspects, the method further comprises assaying the sample for spindle assembly checkpoint (SAC) activation, centrosome aberrations, multipolar spindles or a combination thereof. Suitable methods of assaying the sample for these characteristics/features are described herein. See, Examples 5-10 of WO 2021/211549, the contents of which are incorporated herein by reference.

Any and all possible combinations of the steps described herein are contemplated for purposes of the inventive methods

### Methods of Treatment

Additionally provided herein are methods of treating a neoplastic disease in a subject.

As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a neoplastic disease of the present disclosure can provide any amount or any level of treatment. Furthermore, the treatment provided by the methods of the present disclosure can include treatment of one or more conditions or symptoms or signs of the neoplastic disease being treated. Also, the treatment provided by the methods of the present disclosure can encompass slowing the progression of the neoplastic disease. For example, the methods can treat neoplastic disease by virtue of enhancing the T cell activity or an immune response against the neoplastic disease, reducing tumor or cancer growth or tumor burden, reducing metastasis of tumor cells, increasing cell death of tumor or cancer cells or increasing tumor regression, and the like. In accordance with the foregoing, provided herein are methods of reducing tumor growth or tumor burden or increasing tumor regression in a subject. In exemplary embodiments, the method comprises administering to the subject a KIF18A inhibitor optionally in combination with an agent which lowers APC/C activity or increases SAC activity. terms “treat”, “treating” and “treatment” as used herein refer to therapy, including without limitation, curative therapy, prophylactic therapy, and preventative therapy. Prophylactic treatment generally constitutes either preventing the onset of disorders altogether or delaying the onset of a pre-clinically evident stage of disorders in individuals.

In various aspects, the methods treat by way of delaying the onset or recurrence of the neoplastic disease by at least 1 day, 2 days, 4 days, 6 days, 8 days, 10 days, 15 days, 30 days, two months, 3 months, 4 months, 6 months, 1 year, 2 years, 3 years, 4 years, or more. In various aspects, the methods treat by way increasing the survival of the subject. In exemplary aspects, the methods of the present disclosure provide treatment by way of delaying the occurrence or onset of metastasis. In various instances, the methods provide treatment by way of delaying the occurrence or onset of a new metastasis. Accordingly, provided herein are methods of delaying the occurrence or onset of metastasis in a subject with cancer.

In exemplary instances, the treatment provided may be described in terms of or supported by data obtained from a clinical trial wherein the endpoints of the trial are progression-free survival (PFS), overall survival (OS), or time to deterioration of Eastern Cooperative Oncology Group (ECOG) performance status. In various aspects, the present disclosure provides a method of increasing PFS, OS, or time to deterioration of ECOG performance status in a subject with a neoplastic disease. As used herein, the term “progression-free survival” or “PFS” means the time a treated patient experiences without cancer getting worse (by whatever measure is being used to measure worsening). The term “overall survival” means how long the patient lives after treatment. ECOG performance status is a grade or score according to a scale used by doctors and researchers to assess a patient's disease, e.g., how the disease is progressing/regressing, how the disease affects the daily living abilities of the patient, and determine appropriate treatment and prognosis. ECOG performance status is determined according to the following criteria:

| SCORE | ECOG |
|---|---|
| 0 | Fully active, able to carry on all pre-disease performance without |
| | restriction |
| 1 | Restricted in physically strenuous activity but ambulatory and |
| | able to carry out work of a light or sedentary nature, e.g., light |
| | house work, office work |
| 2 | Ambulatory and capable of all selfcare but unable to carry out |
| | any work activities. Up and about more than 50% of waking |
| | hours |
| 3 | Capable of only limited selfcare, confined to bed or chair more |
| | than 50% of waking hours |
| 4 | Completely disabled. Cannot carry on any selfcare. Totally |
| | confined to bed or chair |
| 5 | Dead |

Oken et. al., Am. J. Clin. Oncol 5: 649-655 (1982)

In exemplary embodiments, the method of treating a subject having a neoplastic disease comprises (I) assaying a sample obtained from the subject for (a) SAC activity, (b) ploidy (c) whole genome doubling (WGD), (d) Anaphase Promoting Complex (APC/C) activity, or (e) a combination thereof and (II) administering a KIF18A inhibitor to the subject when the sample is positive for (a) increased SAC signaling or SAC activity, (b) high ploidy, (c) WGD, (d) low APC/C activity, (e) or a combination thereof as assayed in (I), optionally, wherein the method further comprises obtaining the sample from the subject.

In exemplary embodiments, the method is a method of treating a subject having a neoplastic disease, wherein the subject comprises cells that are positive for (a) increased SAC signaling or SAC activity, (b) high ploidy, (c) WGD, (d) low APC/C activity, (e) or a combination thereof. In exemplary embodiments, the method comprises administering a KIF18A inhibitor to the subject.

In exemplary embodiments, the method is a method of treating a subject with a cancer comprising one or more whole genome duplication or whole genome doubling (WGD) events. In exemplary aspects, the method comprises: (a) assaying APC/C activity in a tumor cell obtained from the subject; and (b) administering to the subject a KIF18A inhibitor when the APC/C activity measured in (a) is low.

In exemplary embodiments, the method is a method of treating a subject with a cancer comprising one or more whole genome duplication or whole genome doubling (WGD) events, and the method comprises (a) lowering APC/C activity in the subject, optionally, by inhibiting expression of UBE2S; and (b) administering to the subject a KIF18A inhibitor. Provided herein is a method of treating a subject with a cancer comprising one or more whole genome duplication or whole genome doubling (WGD) events. In exemplary embodiments, the method comprises (a) administering to the subject an agent that lowers APC/C activity in the subject; and (b) administering to the subject a KIF18A inhibitor.

In exemplary aspects, the KIF18A inhibitor is administered to the subject daily (1 time per day, 2 times per day, 3 times per day, 4 times per day, 5 times per day, 6 times per day), three times a week, twice a week, every two days, every three days, every four days, every five days, every six days, weekly, bi-weekly, every three weeks, monthly, or bi-monthly. In various instances, the CDK inhibitor is administered once daily to the subject. Optionally, the KIF18A inhibitor is administered orally once a day.

Methods of inducing or increasing tumor regression in a subject with a tumor are additionally provided herein. In exemplary embodiments, the method comprises administering to the subject a KIF18A inhibitor in an amount effective to induce or increase tumor regression. The present disclosure also provides methods of reducing tumor growth or cancer growth in a subject.

In exemplary embodiments, the method comprises administering to the subject a KIF18A inhibitor in an amount effective to reduce tumor or cancer growth. Methods of inducing or increasing death of tumor cells or cancer cells in a subject are provided herein. The method in exemplary embodiments comprises administering to the subject a KIF18A inhibitor in an amount effective to induce or increase death of the tumor cells or cancer cells. In various aspects, the neoplastic disease is a cancer, optionally, breast cancer, ovarian cancer, or prostate cancer. In various instances, the neoplastic disease is triple-negative breast cancer (TNBC), non-luminal breast cancer, or high-grade serous ovarian cancer (HGSOC). In exemplary aspects, the neoplastic disease is an endometrial cancer, optionally, serous endometrial cancer. Optionally, the cancer comprises cells that are positive for (a) increased SAC signaling or SAC activity, (b) high ploidy, (c) WGD, (d) low APC/C activity, (e) or a combination thereof. Optionally, the KIF18A inhibitor is administered for oral administration, optionally once a day. In exemplary aspects, the amount of the KIF18A inhibitor is effective induce at least 50% or at least 75% (e.g., at least 80% or 85% or at least 90% or 95%) tumor regression, compared to a control.

### Neoplastic Disease

As used herein, the term “neoplastic disease” refers to any condition that causes growth of a tumor. In exemplary aspects, the tumor is a benign tumor. In exemplary aspects, the tumor is a malignant tumor. In various aspects, the neoplastic disease is cancer. The cancer in various aspects is acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, or urinary bladder cancer. In particular aspects, the cancer is head and neck cancer, ovarian cancer, cervical cancer, bladder cancer, oesophageal cancer, pancreatic cancer, gastrointestinal cancer, gastric cancer, breast cancer, endometrial cancer, colorectal cancer, hepatocellular carcinoma, glioblastoma, bladder cancer, lung cancer, e.g., non-small cell lung cancer (NSCLC), or bronchioloalveolar carcinoma. In particular embodiments, the tumor is non-small cell lung cancer (NSCLC), head and neck cancer, renal cancer, triple negative breast cancer, or gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject.

The terms “cancer” and “cancerous” when used herein refer to or describe the physiological condition in mammals that is typically characterized by unregulated cell growth. Examples of cancer include, without limitation, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, colon carcinoma, and head and neck cancer, ovarian cancer, and endometrial cancer. While the term “cancer” as used herein is not limited to any one specific form of the disease, it is believed that the methods of the invention will be particularly effective for cancers which are found to be accompanied by unregulated levels of KIF18A or dependent on KIF18A for proper chromosome segregation and survival in the mammal.

In various aspects, the cancer is metastatic, the tumor is unresectable, or a combination thereof. In exemplary aspects, the neoplastic disease is a breast cancer, optionally, luminal breast cancer or TNBC. In various aspects, the neoplastic disease is a cancer comprising one or more whole genome duplication or whole genome doubling (WGD) events. WGD in the context of cancer is discussed in Lens and Hemdema, Nature Reviews Cancer 19:32-45 (2019); Ganem et. al., Current Opinion in Genetics & Development 17, 157-162, and Davoli et. al., Annual Review of Cell and Developmental Biology 27, 585-610.

In exemplary embodiments, the neoplastic disease is a cancer comprising one or more whole genome duplication or whole genome doubling (WGD) events.

### Subjects

In exemplary embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates, Ceboids, or Simoids (monkeys) or of the order Anthropoids (humans and apes). In some aspects, the mammal is a human. In various aspects, the subject has a neoplastic disease, e.g., any one of those described herein. The term “patient”, “subject”, or “mammal” as used herein refers to any “patient”, “subject”, or “mammal” including humans, cows, horses, dogs and cats. In one embodiment of the invention, the mammal is a human.

### Samples

With regard to the methods disclosed herein, the sample comprises a bodily fluid, including, but not limited to, blood, plasma, serum, lymph, breast milk, saliva, mucous, semen, vaginal secretions, cellular extracts, inflammatory fluids, cerebrospinal fluid, feces, vitreous humor, bone marrow aspirate, peritoneal cavity fluid (e.g., malignant ascites), or urine obtained from the subject. In exemplary aspects, the sample is a composite panel of at least two of the foregoing samples. In some aspects, the sample is a composite panel of at least two of a blood sample, a plasma sample, a serum sample, and a urine sample. In exemplary aspects, the sample comprises blood or a fraction thereof (e.g., plasma, serum, fraction obtained via leukopheresis). In various aspects, the sample comprises cancer cells, tumor cells, non-tumor cells, blood, blood cells, or plasma. In exemplary instances, the sample comprises cell-free DNA (cfDNA). In exemplary instances, the sample comprises germline cells of the neoplastic disease (e.g., cancer). In exemplary instances, the sample comprises somatic cells of the neoplastic disease (e.g., cancer).

### Controls

In the methods described herein, the level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, BMI, current therapeutic regimen status, medical history, or a combination thereof, but differs from the subject being diagnosed in that the control does not suffer from the disease in question or is not at risk for the disease.

Relative to a control level, the level that is determined may an increased level. As used herein, the term “increased” with respect to level (e.g., expression level, biological activity level) refers to any % increase above a control level. The increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level which is synonymous with a low level. As used herein, the term “decreased” with respect to level (e.g., expression level, biological activity level) refers to any % decrease below a control level. The decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The following examples are given merely to illustrate the present invention and not in any way to limit its scope.

### EXAMPLES

### Example 1

This example describes the identification of genes associating with KIF18A inhibitor resistance or with KIF18A inhibitor sensitivity.

Inhibition of KIF18A has been shown to induce tumor regressions in xenograft models of ovarian cancer. As KIF18A inhibitor treatment of OVCAR-3 high-grade serous ovarian cancer (HGSOC) cells showed marked changes in proteins that regulate cell cycle and mitotic progression (cyclin B1, cyclin E1, BubR1, KIF18A) and apoptosis (Mcl-1, cl-PARP), these changes could serve as markers of target engagement in KIF18A inhibitor sensitive cancers. These observations are consistent with KIF18A inhibitor induced mitotic cell arrest and mitotic cell death, excessive pericentrin spotting, and KIF18A protein mis-localization in mitosis. A subset of TP53 mutant triple negative breast cancer (TNBC) and HGSOC cells are dependent on KIF18A motor activity for proper chromosome alignment and segregation, and KIF18A inhibition leads to SAC activation and/or aberrate centrosome features resulting in multipolar spindles and apoptosis. These results suggest possible mechanisms of KIF18A inhibitor acting in a cancer setting.

To better understand the genes that potentially regulate KIF18A inhibitor sensitivity vs resistance, genome-wide knockout (KO) screens were carried out with OVCAR-8 and OVCAR-3 ovarian cancer cell lines expressing a nuclease and treated with DMSO or KIF18A inhibitor (Compound C9 at IC30, IC50, IC90 concentrations). After treatment, cells were harvested and genomic DNA extraction and Next-Gen Sequencing (NGS), and screen data analysis was performed using a computational tool. Relative to the DMSO, enrichment of synthetic gene-specific RNAs in KIF18A inhibitor treated cells suggest that KO of the gene increases the overall cell fitness (confers resistance) or depletion of synthetic gene-specific RNAs in KIF18A inhibitor treated cells suggest that KO of the gene decreases the overall cell fitness (confers sensitivity). In this manner, the study identified genes involved in resistance or sensitivity to the KIF18A inhibitor. A number of gene KO hits were associated with KIF18A inhibitor resistance including genes BUB1B, MAD1L1, MAD2L1, TRIP13, TTK, ZW10, KIFC1, ZWINT, CCNB1, TPX2, BUB3, KNTC1, TRRAP, DLGAP5, and MAPRE1. A number of gene KO hits were associated with KIF18A inhibitor sensitivity including genes DYNC1H1, DYNLRB1, KIF18A, KIF23, WDR62, PLK1, UBE2S, UBE2C, KATNB1, CDK1, ANAPC4, TUBA1C, METTL16, SPDL1, and CKAP5. Many of these genes are involved in mitosis and spindle assembly checkpoint (SAC). Comparing between OVCAR-8 and OVCAR-3 screens >50 hits are resistant and >60 hits are sensitive in both cell lines, and the hits are involved in KIF18A relevant pathways (e.g., mitosis and spindle assembly checkpoint). There were many hits specific to either of the cell line and need further exploration. Taken together, these results support mitotic spindle and SAC activation as a biological feature correlative with modulating KIF18A inhibitor sensitivity in cancer cells, such as ovarian cancer cells.

### Example 2

This example demonstrates characteristics of KIF18A inhibitor-sensitive cancer cell lines.

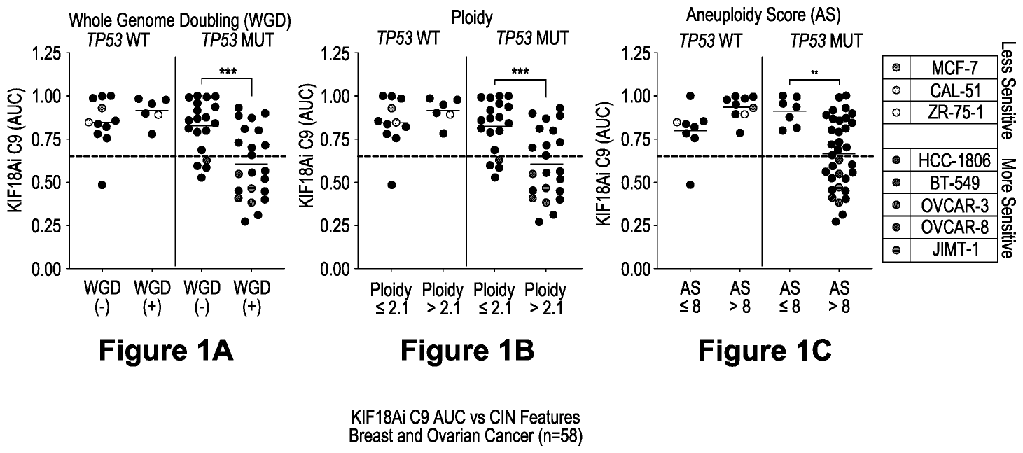

To investigate the anti-proliferative effects of KIF18A inhibitors, a PRISM (profiling relative inhibition simultaneously in mixtures) screen was conducted on a panel of DNA barcoded human cancer cell lines. Pooled cancer cell lines were treated with DMSO or increasing concentrations of KIF18A inhibitor, Compound C9, for 5 days, and then cell viability was determined by measuring the abundance for each unique DNA barcode. We divided the cell lines by CIN features (WGD, ploidy, aneuploidy score) and further subdivided them based on TP53 status. In the TP53^{MUT} cancer cell lines, we observed a significant enrichment for KIF18A inhibitor sensitivity in cell lines that were WGD+ with >2.1 ploidy (55%, 12 of 22 sensitive) relative to WGD− with ≤2.1 ploidy (21%, 4 of 19 sensitive) (p=0.0004). Aneuploidy score was also significantly enriched for KIF18A inhibitor sensitivity in the cell lines with higher aneuploidy scores >8 (47%, 16 of 34 sensitive) relative to lower aneuploidy scores≤8 (0%, 0 of 7 sensitive) (p=0.0037). See FIG. 1.

Taken together, these results suggest that aneuploidy/ploidy and/or WGD status is correlative with KIF18A inhibitor sensitivity in cancer cells.

### Example 3

This example demonstrates KIF18A inhibitor toxicity is caused by mitotic spindle assembly checkpoint (SAC) activation.

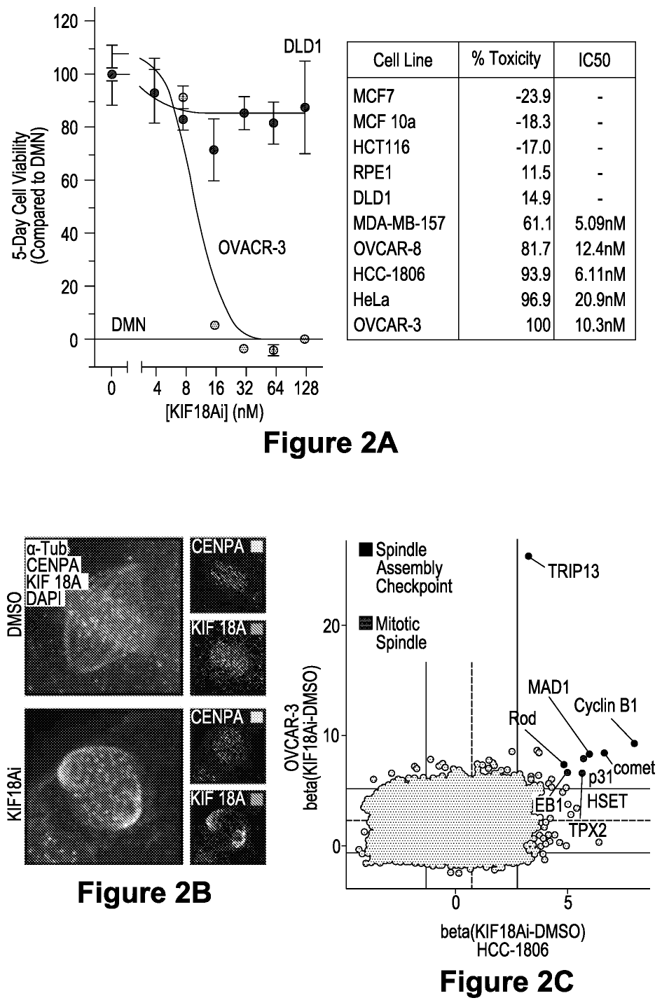

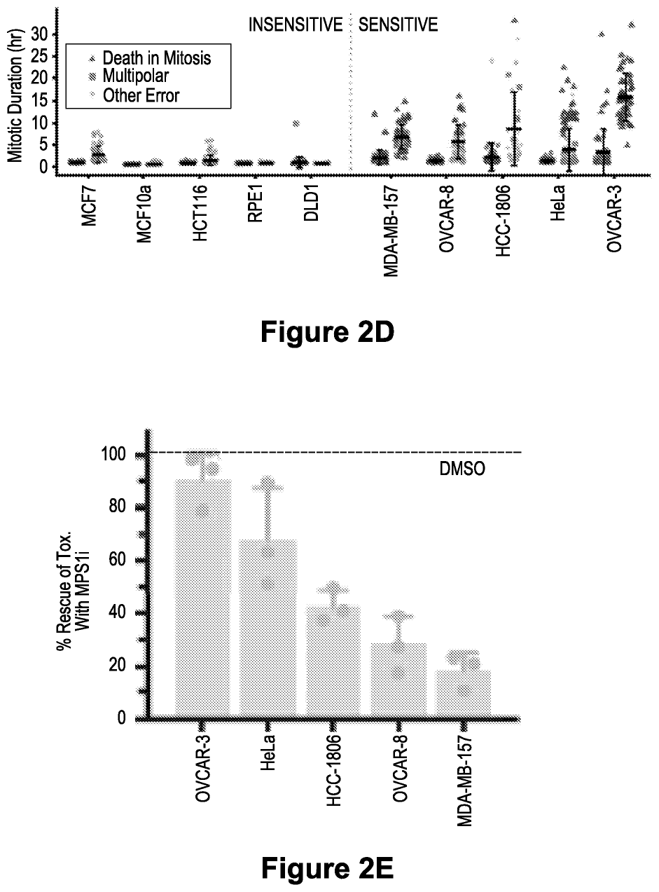

A panel of cell lines both sensitive and insensitive to KIF18Ai (FIG. 2A) were assembled. KIF18A is a plus end directed microtubule motor, and in mitosis, KIF18A travels to the kinetochore where it dampens microtubule dynamics and stabilizes the metaphase plate. Inhibition of KIF18A causes re-localization of the protein to spindle poles where it is presumably unable to complete this function (FIG. 2B). Cells lacking KIF18A have been reported to activate the Spindle Assembly Checkpoint (SAC) even at kinetochores attached to the mitotic spindle, suggesting a spindle tension defect (Janssen et al., Current Biol 28 (17): 2684-2696 (2018)). To identify regulators of KIF18A dependency, whole-genome CRISPR-Cas knockout screens were run in two sensitive cell lines (OVCAR-3 and HCC-1806) with KIF18Ai. Knockout of SAC components as well as mechanical regulators of the mitotic spindle drove resistance to KIF18Ai in these screens (FIG. 2C).

Consistent with SAC activation causing toxicity, KIF18Ai treatment of sensitive cell lines led to a dramatic increase in mitotic duration accompanied by a wide range of mitotic errors. In contrast, insensitive cell lines rarely experienced mitotic delays or errors (FIG. 2D). Partial inhibition of the SAC using an MPS1 inhibitor also rescued much of the drug toxicity in a longitudinal growth assay (FIG. 2E).

### Example 4

This example demonstrates KIF18A inhibition leads to a mild SAC-silencing defect at all kinetochores, independently of attachment.

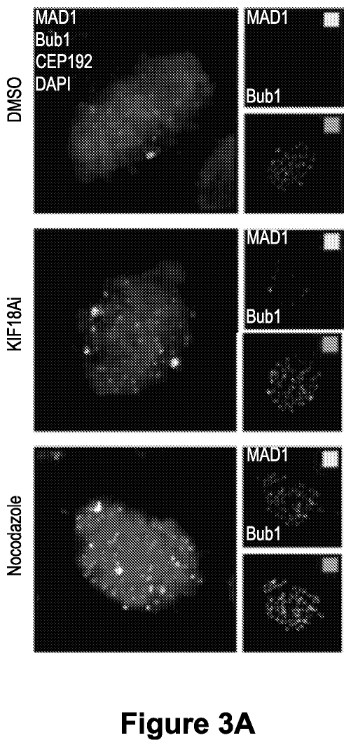

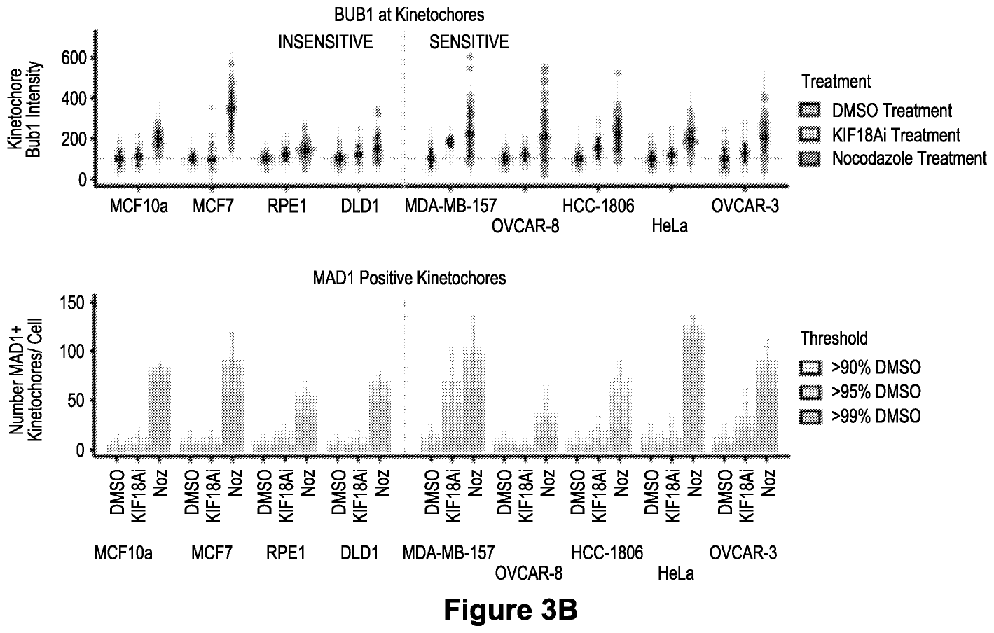

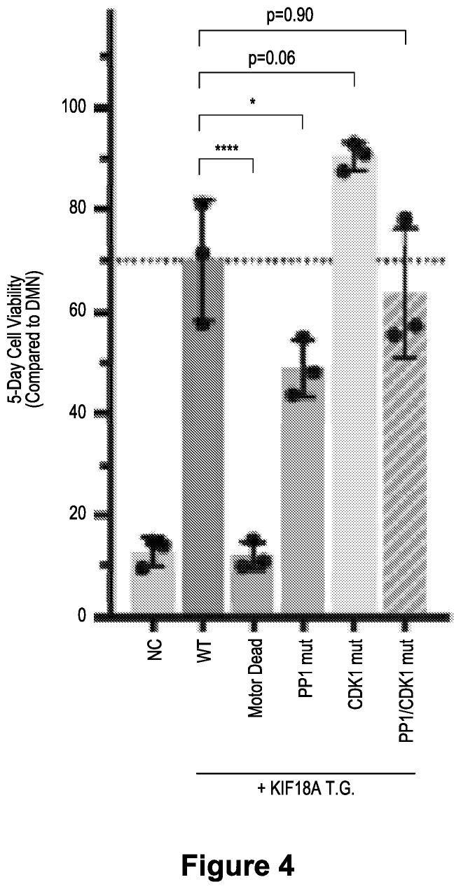

SAC activity is generated at kinetochores that fail to attach and biorient along the mitotic spindle. This led us to hypothesize that KIF18Ai may be preventing mitotic progression through faulty attachments at a small number of kinetochores, which can be visualized by the presence of SAC components including Bub1 and MAD1. Despite apparent attachment and formation of a metaphase plate in KIF18Ai, nearly all kinetochores demonstrated partial SAC activation in immunofluorescence experiments. (FIG. 3). This effect was present in both insensitive and sensitive cell lines suggesting that KIF18Ai toxicity is determined downstream of the SAC and informed by an additional cellular context.

### Example 5

This example demonstrates KIF18A inhibitor recruitment of PP1 is not responsible for the SAC silencing defect.

KIF18A harbors binding sites for the phosphatase PP1, a negative regulator of the SAC, and the mitotic kinase CDK1. In addition, these proteins play antagonistic roles on KIF18A motor activity4. Using sequence alignment with known off-target kinesins as well as protein-drug structural information, we generated a KIF18Ai-resistant KIF18A transgene. Mutation of either or both PP1 and CDK1 binding sites revealed that KIF18A dependency is not mediated through a loss of PP1 or CDK1 recruitment to the kinetochore and is more likely caused by a loss of microtubule plus end dampening.

### Example 6

This example demonstrates low APC/C activity sensitizes WGD cancer cells to KIF18A inhibition.

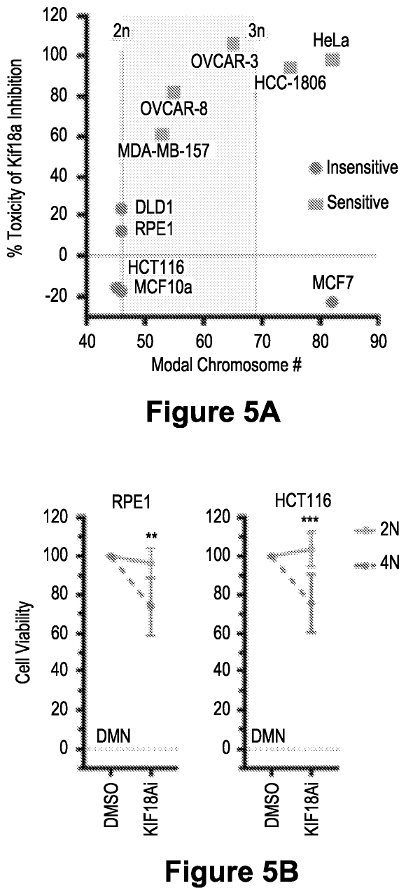

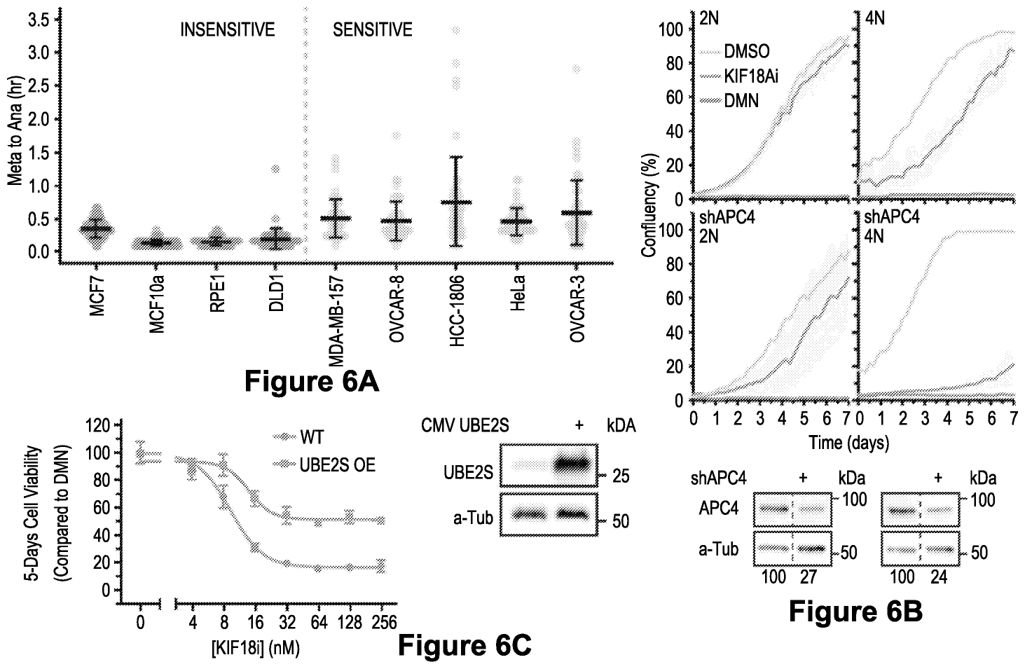

Whole genome doubling (WGD) has previously been identified as a driver of KIF18A dependency, consistent with a ploidy-based amplification of a pan-kinetochore SAC-silencing defect. However, ploidy is a weak correlate for KIF18Ai toxicity, and synthetic WGD of a pair insensitive diploid cell lines only mildly sensitized these cells to KIF18Ai (FIG. 5). Curiously, sensitive cancer cell lines appeared deficient in APC/C activity as reported by a delayed metaphase-to-anaphase transition (FIG. 6A). Lowering APC/C activity in insensitive diploid cells using an shRNA against APC4 or its E2 ligase UBE2S showed only modest effects, however combining both low APC/C activity with WGD lead to penetrant cell death in the inhibitor (FIG. 6B). Excitingly, increasing APC/C activity by overexpression of UBE2S partially rescued toxicity in sensitive HCC-1806 cells (FIG. 6C). This suggests low APC/C is indeed responsible for sensitizing these cells to KIF18Ai.

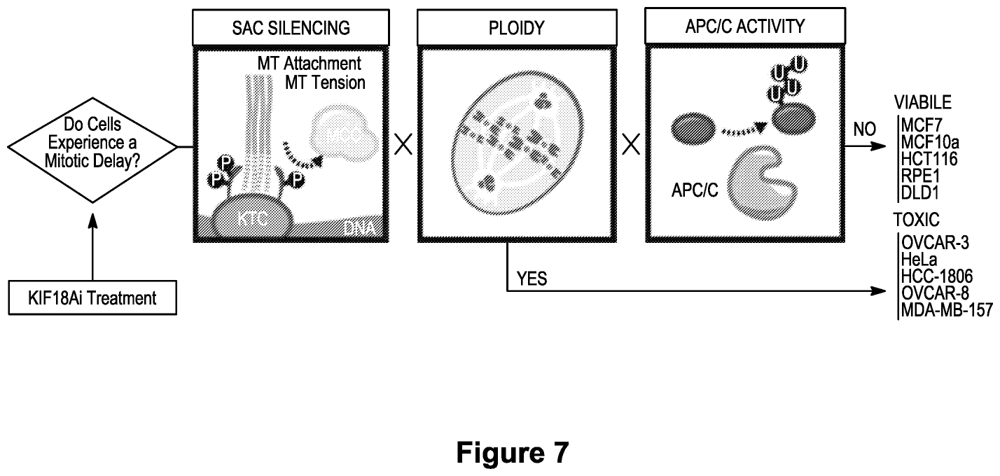

Taken together, the results presented in the examples suggest dependency on KIF18A is caused by a failure of high ploidy, low APC/C activity cells to respond to a kinetochore-wide SAC silencing defect. A schematic of sensitivity of KIF18Ai is shown in FIG. 7. Here it is suggested that SAC activity, ploidy and/or APC/C activity are factors that should be considered when determining whether KIF18Ai treatment leads to toxicity in cancer cells. Such schematic can assist in identifying cancers as treatable with a KIF18A inhibitor.

### Example 7

This example describes the materials and methods used in Examples 3-6.

### Spindle Assembly Checkpoint Immunofluorescence Assay

Cell lines were plated at 40% confluence on glass coverslips the day before the experiment. The following day, 10 mM MG132 (proteasome inhibitor) was added simultaneously with 3.3 uM Nocodazole, 250 nM KIF18A inhibitor C9, or DMSO from 1000× stocks and incubated for 4 hr. Cells were rinsed once with 1×PBS, 30s with Microtubule Stabilizing Buffer (30% glycerol, 100 mM PIPIES, 1 mM EDTA, 1 mM MgSO4), once with PBS, then fixed for 10 min with 4% PFA in PBS. After fixation, cells were blocked in Triton block for 1 hr then probed for Bub1 (1:1000, Sh-a-Bub1, gift of S. Taylor #SRB1.1), MAD1 (1:1000, M-a-MAD1, Sigma #MABE867), CEP192 (1:1000, Alexa555-R-a-CEP192, in house from ProSci), and DAPI using standard methods. >30 Mitotic cells were imaged for each condition.

Analysis of images was performed using a custom imagej script. Cells at metaphase were first identified for analysis. Individual kinetochores were segmented in 3D from the Bub1 signal. Measurements were made for Bub1 and MAD1 within each kinetochore region. Quality control of kinetochores was performed using a custom R script, by excluding kinetochores outside a pre-determined volume (noise or merged kinetochores), signal that overlapped with CEP192 (spindle poles), or cells outside an appropriate range for kinetochore number. All data was normalized to the DMSO condition. MAD1+kinetochores were identified as kinetochores with MAD1 signal surpassing the indicated percentile of the DMSO condition.

### Drug-Resistant Transgene

CMV KIF18A-(3×) HA was transduced into cells with a lentiviral vector and selected using 1 ug/mL puromycin. All transgenic constructs included the drug-resistant mutation (G289I ) in addition to PP1, CDK1, and motor mutations described in (Häfner, J., et al. Nat Commun 5, 4397 (2014)) and (Stumpff, J., et al. Dev Cell 14:2, 252-262 (2008)). All assays were run in triplicate according to the MTT cell viability assay.

### Toxicity of KIF18A Inhibitor

Cell line toxicity was determined using the MTT cell viability assay (1−(% Viability_{250nMKIF18Aic9})/(% Viability_{DMSO}))×100. Modal chromosome number was used from ATCC. 2N and 4N RPE1 and HCT116 cells were a gift of N. Ganem as described in (Ganem N J, et al., Nature 460, 278, (2009)).

### APC/C Activity of Cells

Metaphase-to-Anaphase duration was determined from live cell imaging of transgenic cell lines expressing H2B-iRFP.

Cell growth was monitored in triplicate from the indicated cells plated at 800 cells/well in a 96-well plate using a CellcyteX for 7 days. Cells were treated with either 250 nM KIF18Ai C9, 2 uM Dimethylenastron, or DMSO from 200× stock. APC4 was knocked down using GIPZ shRNA (horizon, V2LHS_254660, mature antisense 5′-TACAATGGAATACAGATTG-3′) and validated by Western blot using standard protocols (1:1000, R-a-APC4, Bethyl Laboratories #A301-176A).

CMV UBE2S was transduced into cells with a lentiviral vector, selected using 1 ug/ml puromycin, and validated by Western blot using standard protocols (1:1000, R-a-UBE2S, Proteintech #14115-1AP)

### MTT Cell Viability Assay:

Cells were plated according to the following table in a 96-well plate and left to grow overnight. KIF18Ai C9 was added from 1:200 stock in DMSO at the indicated concentration or 250 nM if not indicated in duplicate or triplicate. Control wells were included with either DMSO or 2 uM Dimethylenastron. Cells were grown for 5 days in drug. 250 ug/mL MTT (3-(4,5-Dimethylthiazol-2-yl)-2,5-Diphenyltetrazolium Bromide) was added to cells for 4.5 hr, then cells were solubilized by adding 100 uL of 10% SDS in PBS to all wells, then incubated 24 hr at 37C. Absorbance was measured at 570 nM on a plate spectrophotometer.

| Cell line | Cells/well in 96-well plate | Volume (uL) |
|---|---|---|
| RPE1 | 600 | 200 |
| DLD1, HeLa, OVCAR-8, | 1000-1200 | 200 |
| MCF10a, HCC-1806 | | |
| MCF7 | 5000 | 200 |
| OVCAR-3 | 8500 | 200 |
| MDA-MB-157 | 15000 | 200 |

### Example 8

This example demonstrates that SAC activation in cells with reduced APC/C activity drives cancer vulnerability to KIF18A inhibition

### Abstract:

The efficacy of current antimitotic cancer drugs is limited by toxicity in highly proliferative healthy tissues. A cancer-specific dependency on the microtubule motor protein KIF18A therefore makes it an attractive therapeutic target. Not all cancers require KIF18A however, and the determinants underlying this distinction remain unclear. Here we show that KIF18A inhibition drives a modest increase in spindle assembly checkpoint (SAC) signaling which can result in lethal mitotic delays. Whether cells arrest during division depends on a balance between SAC signal strength and anaphase promoting complex/cyclosome (APC/C) activity. Increased chromosome number and low basal APC/C activity dysregulate this balance and cooperatively increase KIF18A dependency. KIF18A dependent cancer cells exhibit hallmarks of this imbalance, including high ploidy, a long metaphase to anaphase transition, and slow mitosis overall. Together, our data reveal vulnerabilities in the cell division apparatus of cancer cells that can be exploited for therapeutic benefit.

### Background:

Cancer is characterized by uncontrolled cell growth. Consequently, the cell cycle and mitosis have long been appealing targets for chemotherapy. Early successes with ‘antimitotic’ drugs emerged from using microtubule targeting agents such as taxanes and vinca alkaloids to disrupt the assembly of the mitotic spindle apparatus and kill dividing cells^{1,2}. Despite decades of widespread usage and efficacy across several tumor types, these drugs suffer from significant limitations: antimitotic drugs also kill healthy, highly proliferative cells of the bone marrow and gut, and significant neurotoxicity is observed due to the disruption of microtubule dynamics in non-dividing neurons³⁻⁷. These drawbacks fueled the development of a new class of drugs that target essential mitotic proteins such as kinases and microtubule motor proteins. This second generation of antimitotic agents mitigates the neurotoxicity observed with microtubule targeting agents. However, myelosuppression remains an unavoidable dose-limiting toxicity in patients^{8,9}, and as a result, these drugs have failed to progress in the clinic.

Because antimitotic agents agnostically kill all dividing cells, identifying and exploiting tumor-specific vulnerabilities in the cell division apparatus remains a major opportunity for drug development. One promising target is the plus-end directed motor protein KIF18A, which is uniquely essential for the division of certain cancers¹⁰⁻¹². KIF18A accumulates at the plus ends of kinetochore microtubules and suppresses chromosome oscillations within the metaphase plate¹³⁻¹⁶. Loss of KIF18A disrupts chromosome alignment, leading to an increased instance of lagging anaphase chromosomes and micronucleation^{10,17}. KIF18A loss has been shown to activate the spindle assembly checkpoint (SAC) at microtubule-attached kinetochores¹⁸, suggesting incomplete microtubule occupancy or tension defects. Despite roles in chromosome alignment and SAC silencing, KIF18A is largely dispensable for accurate chromosome segregation in non-transformed cells¹⁹, and KIF18A knockout mice survive through adulthood²⁰.

KIF18Ai treatment across the large-scale pan-cancer PRISM panel (629 cell lines) revealed that 25% of cancers overall and 32% of ovarian cancers exhibited strong KIF18A dependency²¹. It remains unclear, however, what features distinguish KIF18A-dependent and insensitive cancer cells. Chromosomal instability (CIN) is a common feature of human tumors that results in high rates of chromosome mis-segregation during cell division. It has been proposed that the altered microtubule dynamics in cancers with CIN render them more reliant on KIF18A function¹¹. Separately, whole genome doubling (WGD) and aneuploidy have been shown to increase KIF18A dependency^{10,12}. Since WGD promotes CIN and aneuploidy²², the sensitization mechanisms reported in these studies may be interrelated. Nevertheless, neither WGD nor CIN fully explains sensitivity to KIF18A loss, and we lack a unified mechanistic model for KIF18A dependency.

Here, using a small molecule inhibitor of KIF18A, we demonstrate that long mitotic delays and lethal errors in cell division drive cell death in KIF18A-dependent cancers. KIF18A inhibition halts cells in mitosis by destabilizing kinetochore-microtubule interactions leading to modest SAC activation. This increase in SAC signal is common among sensitive and insensitive cells but is exacerbated by increased ploidy. The anaphase promoting complex (APC/C) is inhibited by the SAC to prevent mitotic exit, and we show cells with low basal APC/C activity are particularly sensitive to modest SAC activation. A combination of heightened SAC signaling and low APC/C activity primes cells for mitotic delay, leading to increased toxicity with KIF18A inhibition. Collectively, our results reveal multiple features contributing to KIF18A dependency that can be used to guide the development of clinical indicators for the efficient use of KIF18A inhibitors.

### Results:

Sensitivity to KIF18A Inhibition is Defined by Long Mitotic Delays that Drive Catastrophic Mitotic Errors

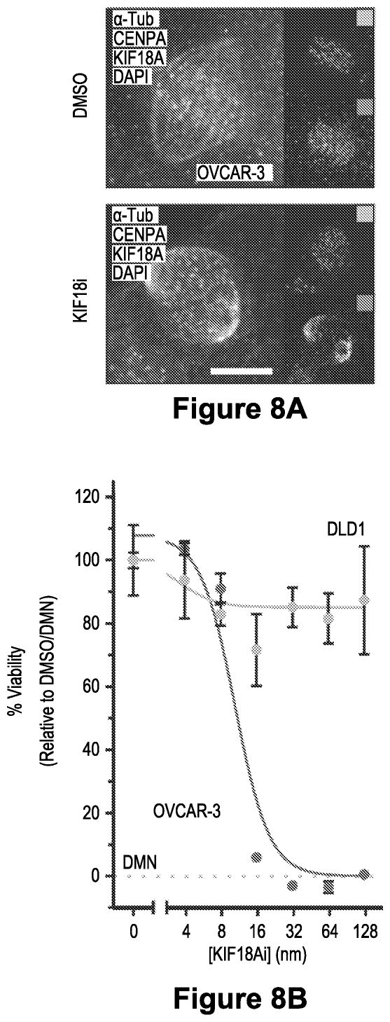

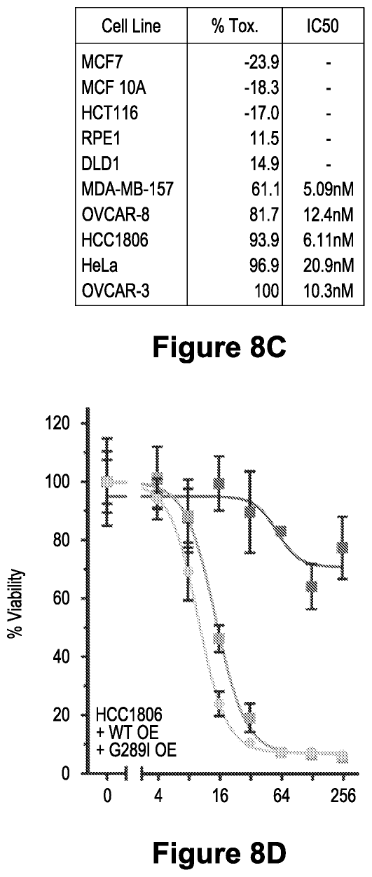

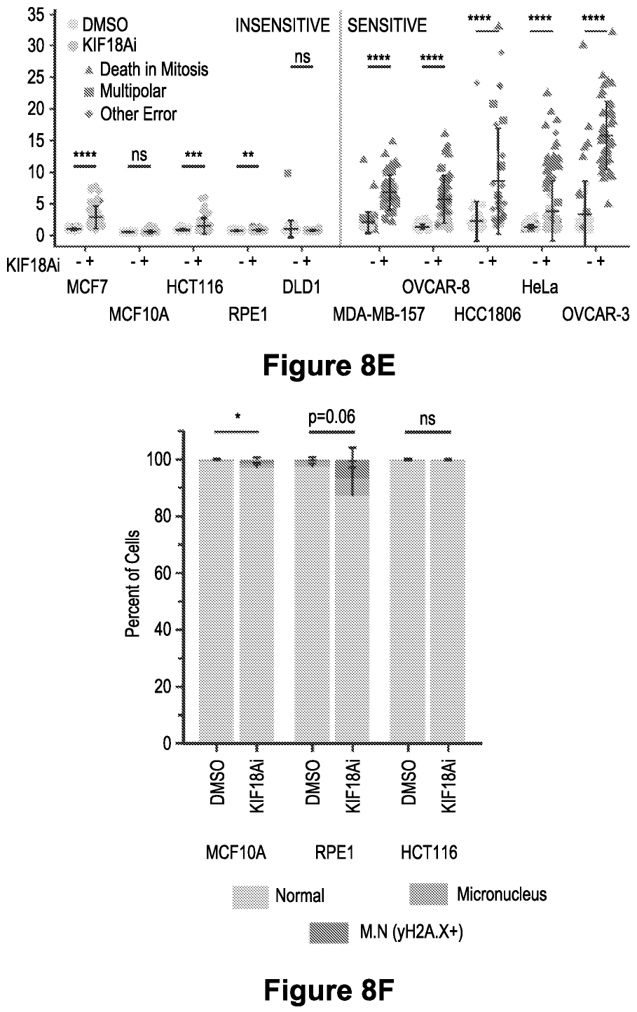

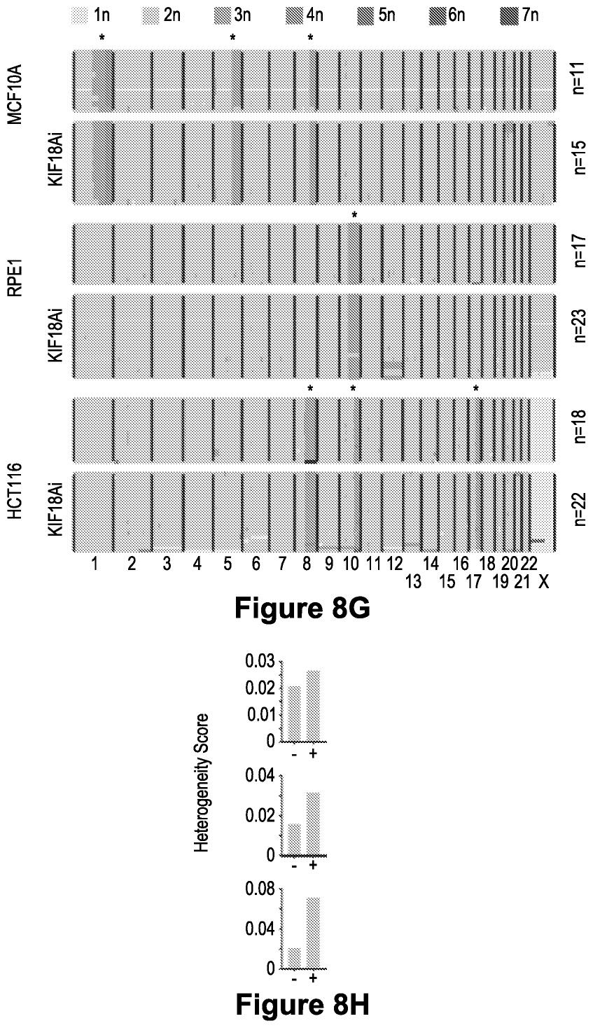

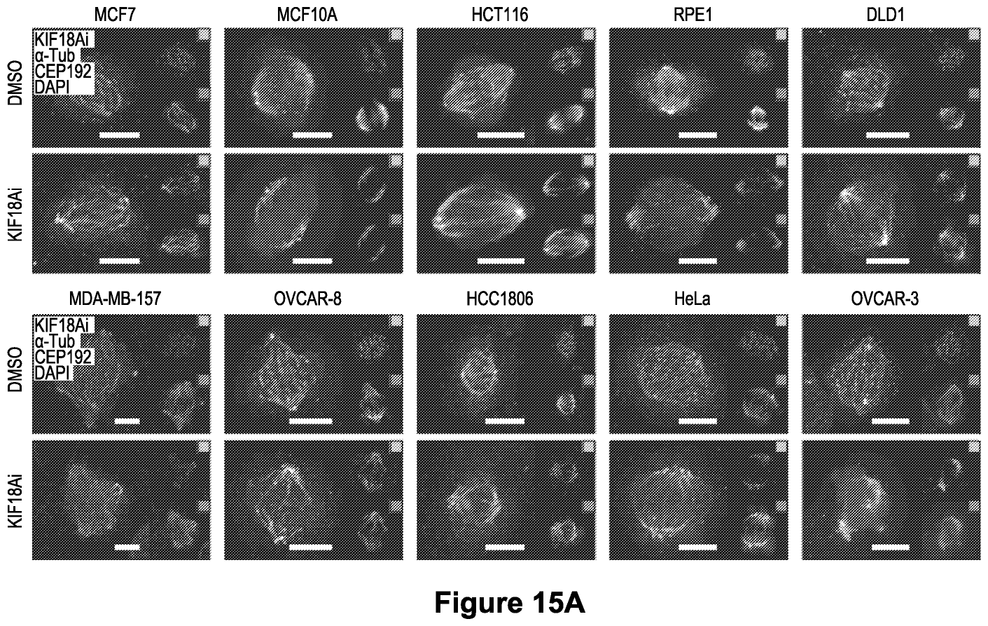

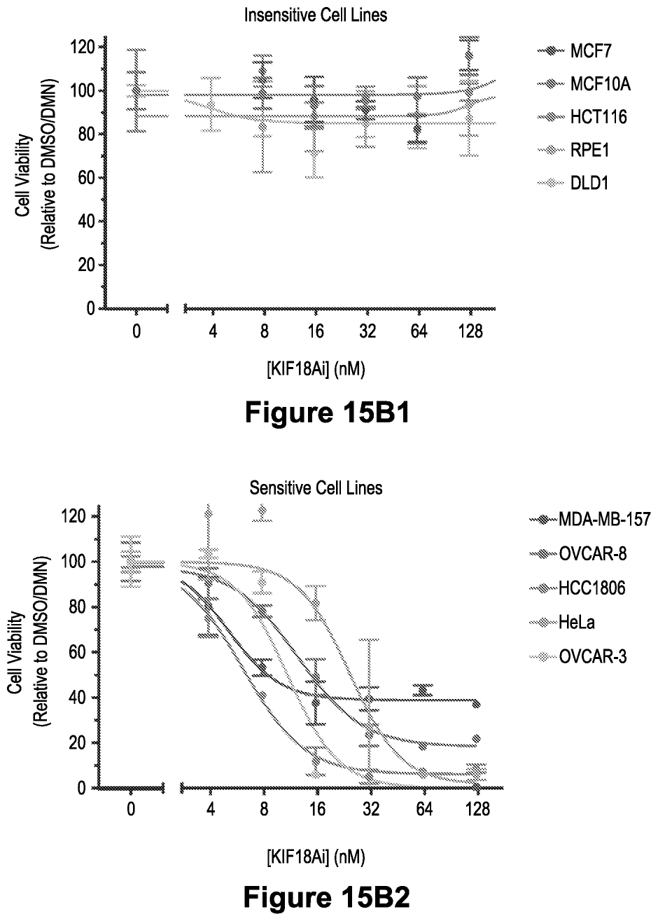

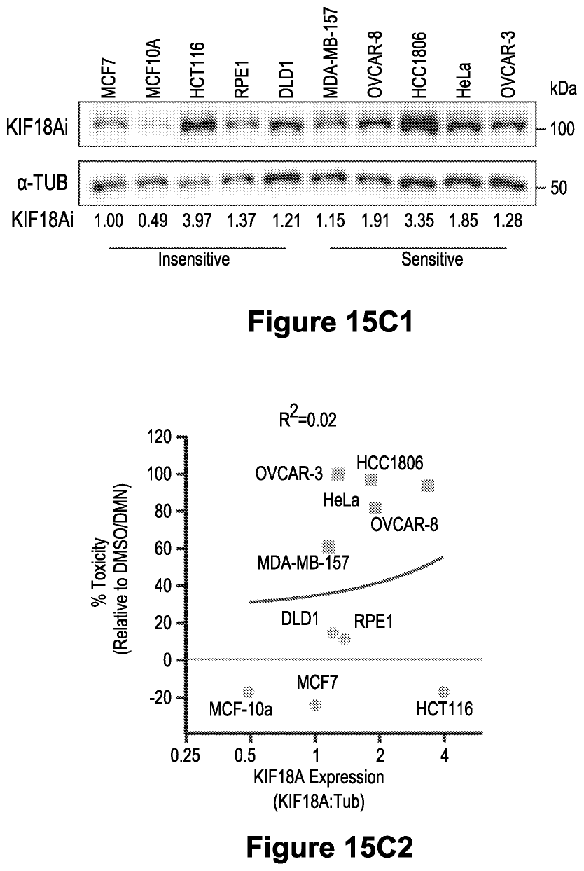

To investigate the determinants of KIF18A dependency in cancer, we used AM-1882, a highly specific small molecule KIF18A inhibitor (KIF18Ai) developed by Amgen Inc^{21,23}. Treatment with KIF18Ai causes stable binding of the KIF18A motor domain to microtubules²³, leading to the relocation of KIF18A in mitosis from kinetochores to spindle poles in all the cell lines tested (FIG. 8a and FIG. 15a). This mis-localization phenocopies the effect of KIF18A loss²¹, likely by blocking KIF18A-induced stabilization of microtubule plus ends¹⁶. We examined the effect of KIF18Ai on cell viability using a 5-day endpoint assay performed with non-transformed cells (RPE1 and MCF10A) and a panel of colon (HCT116, DLD1), breast (MCF7, MDA-MB-157, HCC1806), and ovarian (HeLa, OVCAR-3, OVCAR-8) cancer cell lines (FIG. 8b,c and FIG. 15b). Sensitivity to KIF18Ai varied across the cell line panel and did not correlate with KIF18A expression levels (FIG. 15c). For downstream analysis, we divided cell lines into KIF18Ai sensitive or insensitive: sensitive cell lines exhibited >60% toxicity to KIF18Ai, while insensitive cell lines showed <15% toxicity over a 5-day period.

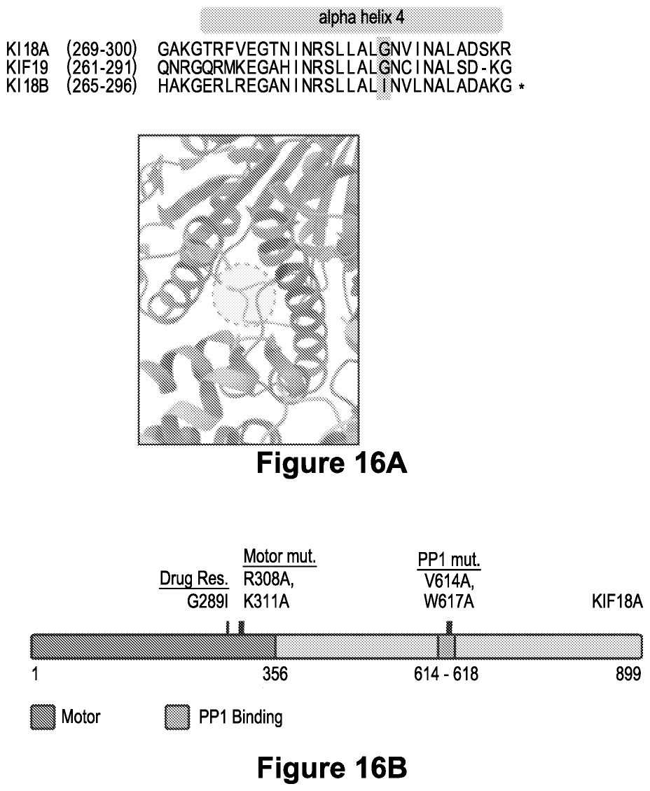

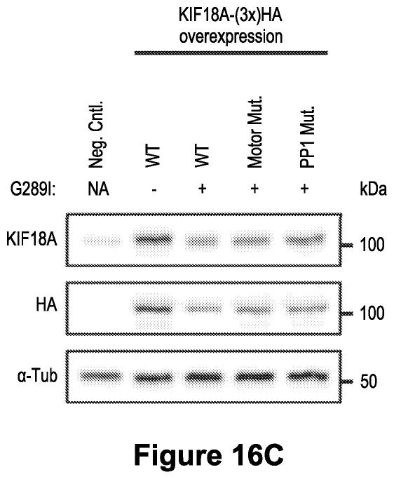

To confirm that the toxicity from KIF18Ai treatment was due to impaired KIF18A function, we set out to generate a drug-resistant KIF18A. KIF18Ai has partial activity toward the related kinesin KIF19 but not KIF18B²¹. Exploiting this distinction, we identified a drug-facing glycine present in the KIF18Ai binding pocket of KIF18A and KIF19 that is changed to isoleucine in KIF18B (FIG. 16a)²³⁻²⁵. We hypothesized that this G:I substitution was the cause of the KIF18Ai resistance for KIF18B. Consistently, overexpression of a KIF18A G289I transgene, but not WT KIF18A, dramatically relieved KIF18Ai toxicity (FIG. 8d and FIG. 16b,c). This demonstrates that KIF18Ai toxicity results from the loss of KIF18A function.

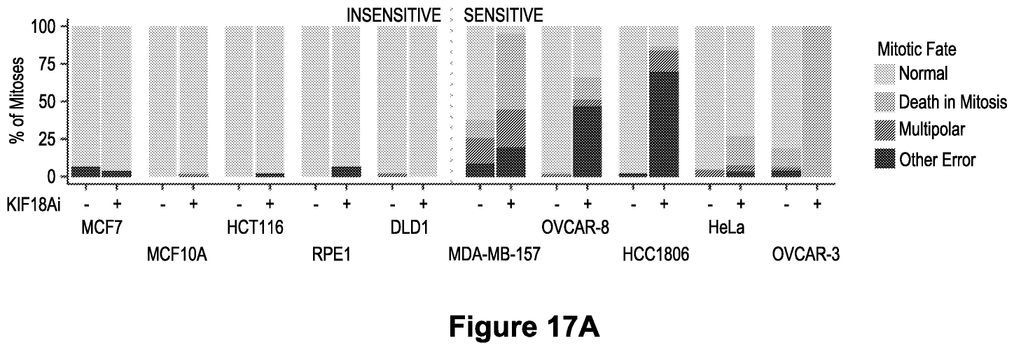

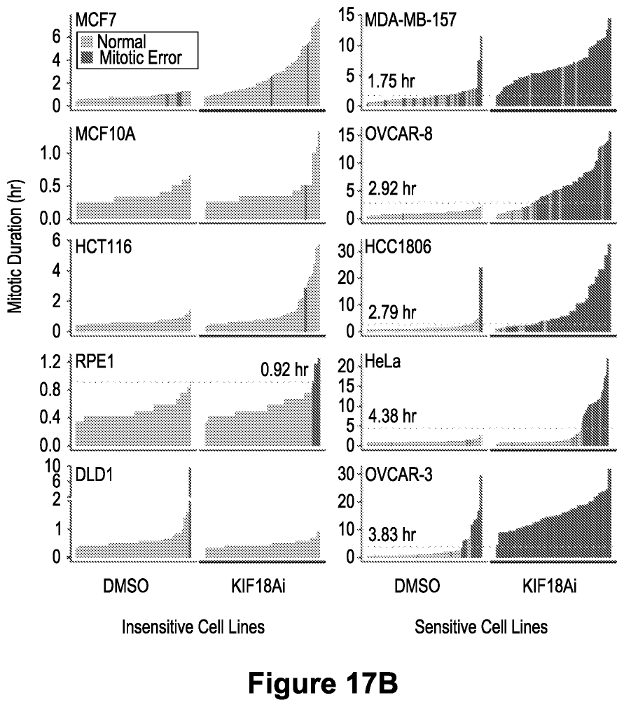

We next evaluated the immediate effects of KIF18A inhibition on mitosis using live-cell imaging of cells labeled with a fluorescent histone H2B and α-Tubulin. While insensitive cells divided mostly normally with KIF18Ai treatment, sensitive cell lines experienced varying degrees of lengthy mitotic delays that often resulted in lethal cell division errors (FIG. 8e). Consistent with KIF18A depletion experiments¹⁴, KIF18Ai treatment also caused chromosome hyper-oscillation and severely misaligned chromosomes were frequently observed. KIF18Ai-sensitive cell lines preferentially underwent either multipolar divisions (MDA-MB-157), chromosome segregation errors (HCC1806), or death in mitosis (MDA-MB-157, OVCAR-8, HeLa, OVCAR-3) (FIG. 17a). Sensitive cell lines also exhibited a narrow threshold of mitotic duration (1.75-4 hr) past which degeneration of the mitotic spindle or a loss of chromosome cohesion occurred (FIG. 17b). Treatment with KIF18Ai extended the duration of mitosis past this threshold leading to spindle degeneration and/or cohesion loss and chronic SAC activation. By contrast, insensitive cell lines treated with KIF18Ai typically passed through mitosis with normal timing or, in the case of MCF7 cells, maintained spindle integrity and chromosome cohesion throughout a modestly prolonged division (FIG. 8e). This suggests that both an increased incidence of, and sensitivity to, mitotic delays are responsible for KIF18Ai toxicity.

Loss of KIF18A has previously been shown to cause low rates of chromosome mis-segregation and micronucleus formation^{10,17}. We therefore investigated whether KIF18Ai-insensitive cell lines also suffered from these defects. A 7-day treatment with KIF18Ai modestly increased the proportion of micronuclei in insensitive RPE1 and MCF10A, but not HCT116 cells (FIG. 8f). We then used whole-cell, single-cell DNA sequencing to monitor changes in karyotype, where a moderate increase in aneuploidy was observed after KIF18Ai treatment in all three cell lines (FIG. 8g,h). We conclude that KIF18Ai treatment leads to mitotic delay and catastrophic mitotic errors in sensitive cell lines. By contrast, KIF18A inhibition in insensitive cell lines results in low rates of chromosome mis-segregation that have a minimal impact on short-term proliferation.

### CRISPR-Cas9 Screens Identify Multiple Pathways for KIF18Ai Resistance

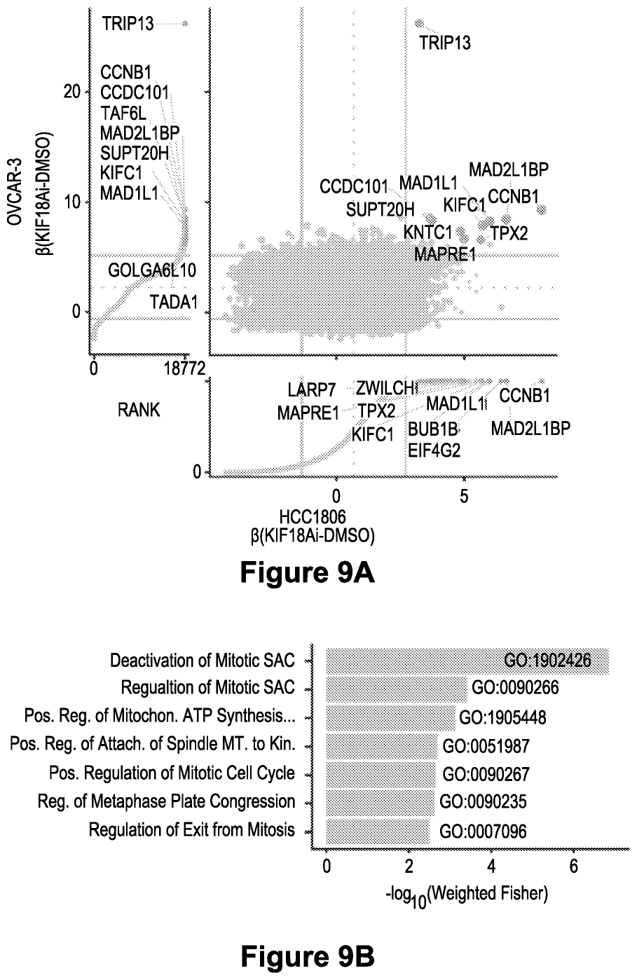

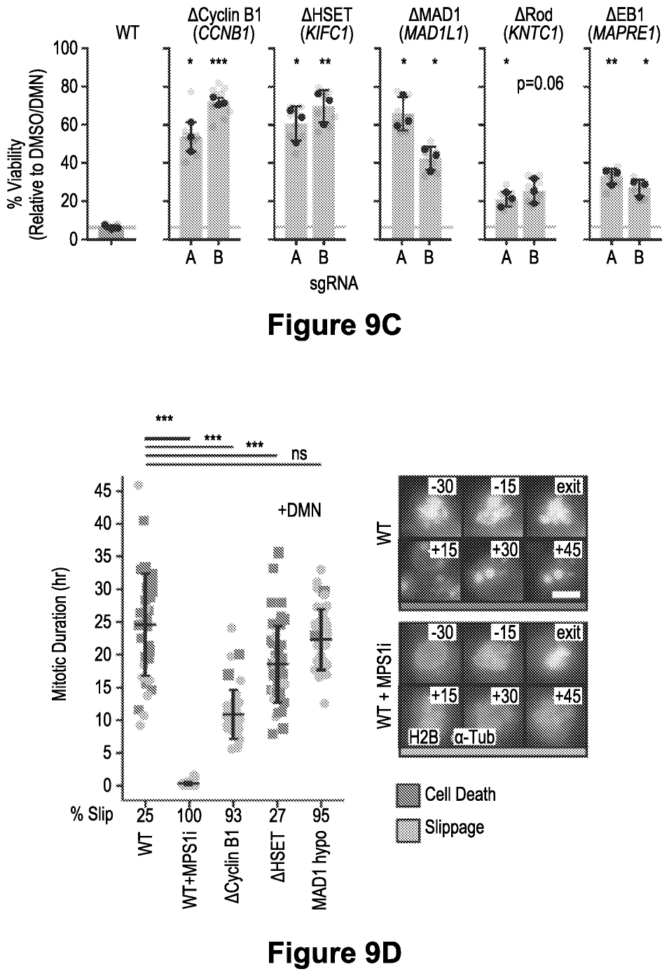

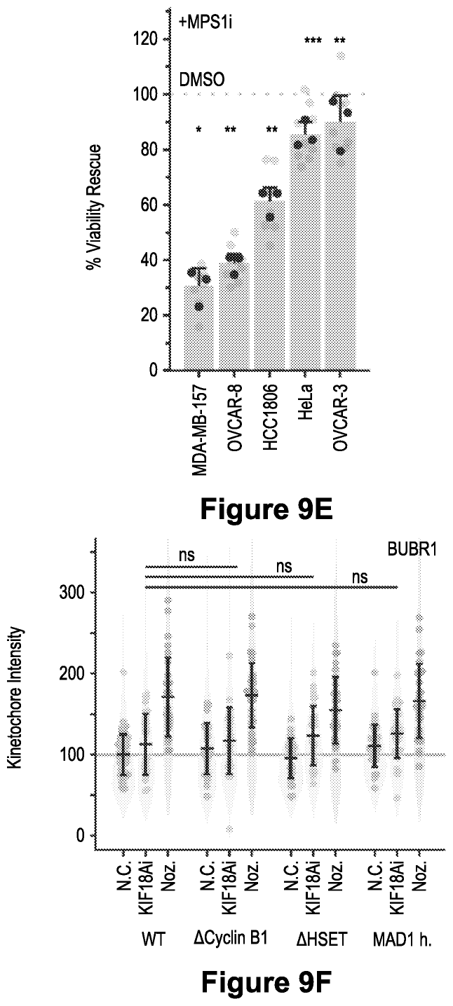

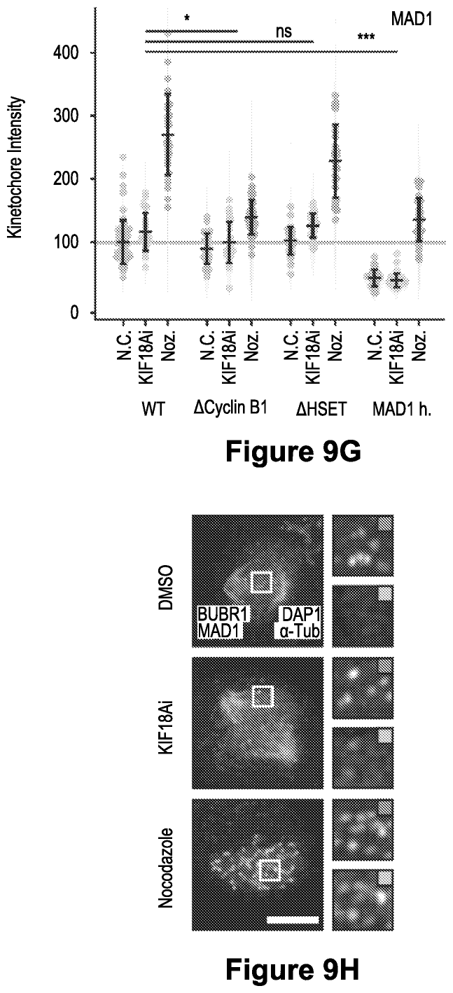

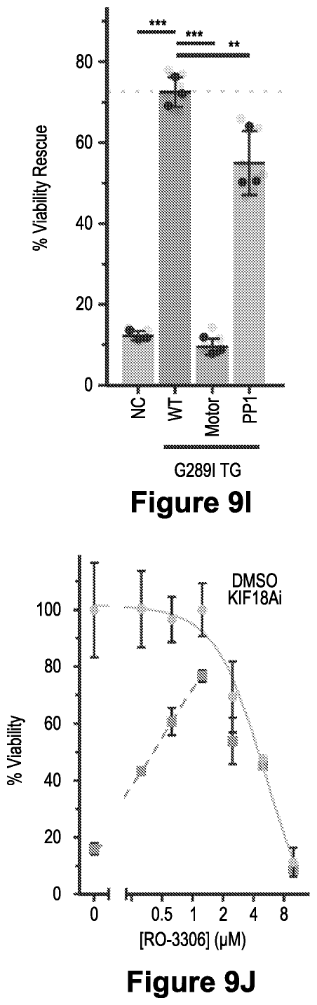

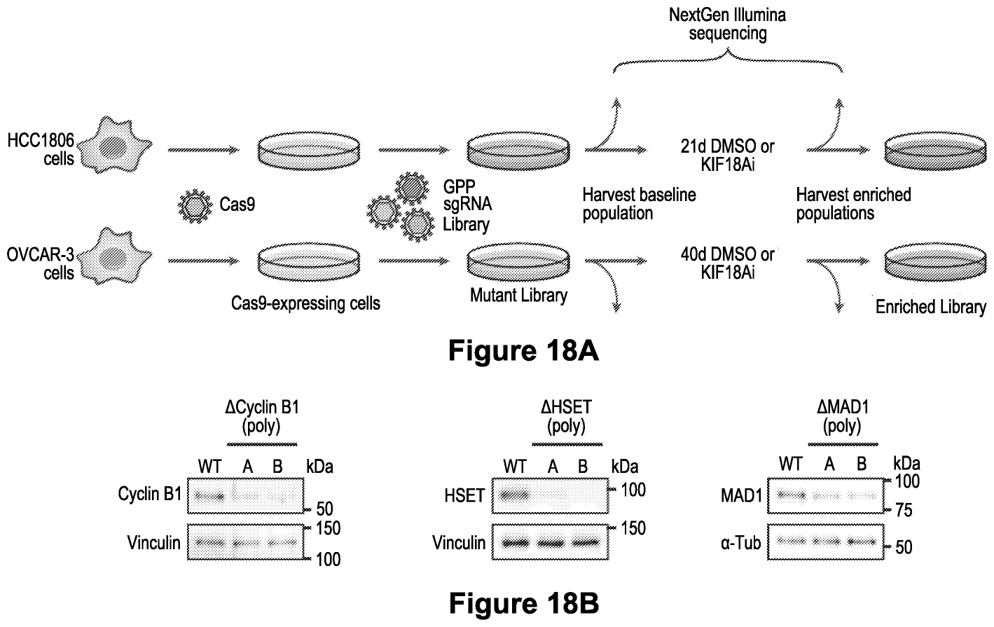

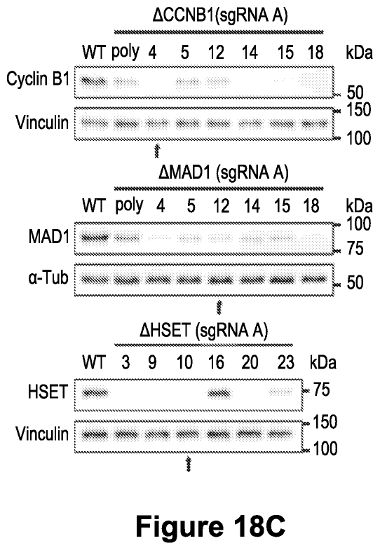

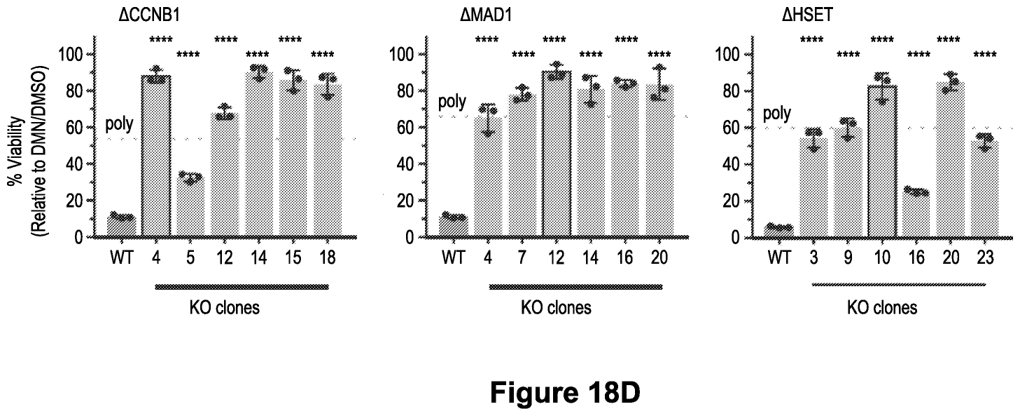

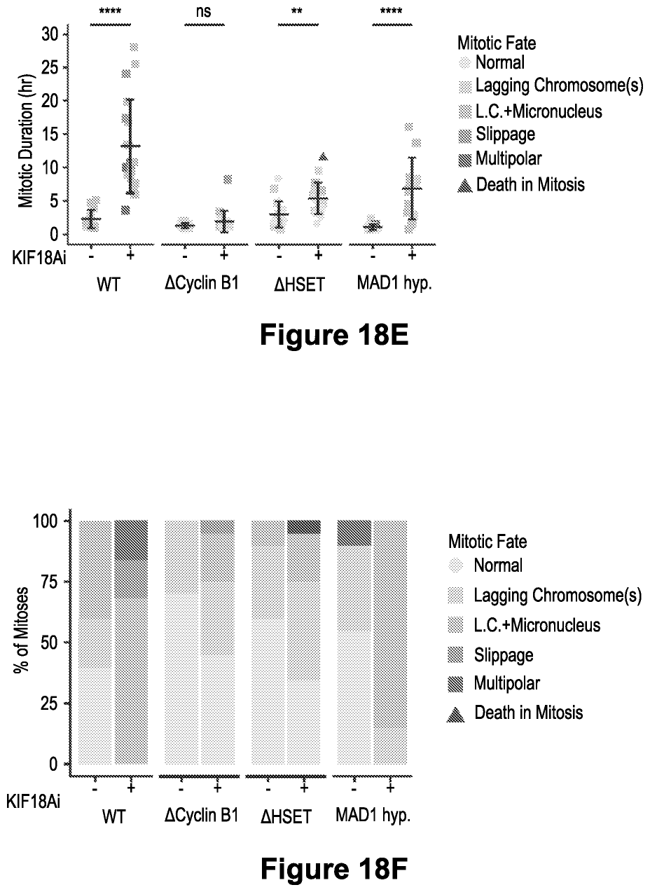

To identify regulators of KIF18A dependency, we performed paired genome-wide CRISPR-Cas9 knockout screens in two highly KIF18Ai-sensitive cell lines: HCC1806 and OVCAR-3. Cas9-expressing HCC1806 and OVCAR-3 cells were transduced with the Brunello sgRNA knockout library and selected for 7 days. Knockout HCC1806 and OVCAR-3 cells were grown in DMSO or KIF18Ai for 21 or 40 days, respectively, and guides that conferred resistance to KIF18Ai were identified by deep sequencing (FIG. 18A). We predicted that two classes of genes would drive resistance to KIF18Ai: genes whose loss reduced the barrier to mitotic exit, and genes whose loss directly corrected the mitotic defects caused by KIF18A inhibition. In accordance with this expectation, SAC components and microtubule regulators were highly enriched in the screen hits (FIG. 9a,b). We focused on five hits with roles in the SAC (MAD1, Rod), mitotic spindle dynamics (HSET, EB1), or both (Cyclin B1). Independent polyclonal knockout HCC1806 cells expressing the top-performing sgRNAs for these genes demonstrated strong KIF18Ai resistance (>60%) for MAD1, HSET, and Cyclin B1 and modest resistance (<30%) for Rod and EB1 (FIG. 9c). We, therefore, focused on defining the role of MAD1, HSET, and Cyclin B1 loss in conferring KIF18Ai resistance.

The Broad DepMap, a database which reports pan-genome CRISPR-Cas9 knockout sensitivity data for over 1000 cancer cell lines, lists Cyclin B1 and MAD1 as common essential genes. Consistently, residual protein was detectable in the polyclonal HCC1806 Cyclin B1 and MAD1 sgRNA-expressing populations (FIG. 18b). We therefore derived clonal cell lines edited for Cyclin B1, MAD1, and HSET. Though we isolated multiple clones without detectable Cyclin B1 or HSET protein, all MAD1 clones retained low levels of MAD1 protein expression, suggesting that a complete loss of MAD1 is lethal in HCC1806 cells (FIG. 18c). In all cases, the viability rescue in KIF18Ai correlated well with the degree of Cyclin B1, MAD1 or HSET protein loss detected by western blot (FIG. 18d). In addition, live cell timelapse imaging demonstrated a reduced frequency of severe mitotic errors in response to KIF18Ai treatment in the clonal MAD1, HSET, and Cyclin B1 edited cell lines (FIG. 18e,f).

### SAC Activation Drives KIF18Ai Toxicity

We predicted that depletion of MAD1 or Cyclin B1 might be lowering the barrier to mitotic exit and relieving KIF18Ai toxicity either by interfering with SAC signaling or by reducing the activity of the master mitotic kinase CDK126,27. To define the capacity of KIF18Ai-resistant HCC1806 clonal lines to maintain the mitotic state, we arrested cells indefinitely in mitosis with the Eg5 inhibitor dimethylenastron (DMN) which causes spindle collapse and chronic SAC activation^{28,29}. We then monitored mitotic slippage by live-cell timelapse imaging. DMN treated WT HCC1806 cells stayed in mitosis for an average of 25 hr, resulting in 75% cell death and 25% mitotic slippage (FIG. 9d). By contrast, abolishing SAC function with the MPS1 inhibitor Reversine led to all cells exiting mitosis in less than an hour. Hypomorphic MAD1 cells spent slightly less time in mitosis (23 hr) and had a dramatically increased rate of mitotic slippage (95%). Knockout of Cyclin B1 reduced the time spent in mitosis to 11 hr with similarly high slippage rates (93%), while HSET knockout cell lines spent slightly less time in mitosis (19 hr) but had similar rates of mitotic slippage (27%) compared to control HCC1806 cells. Taken together, these data show that reductions in the level of Cyclin B1 or MAD1 suppress the ability of cells to delay mitosis and induce cell death.

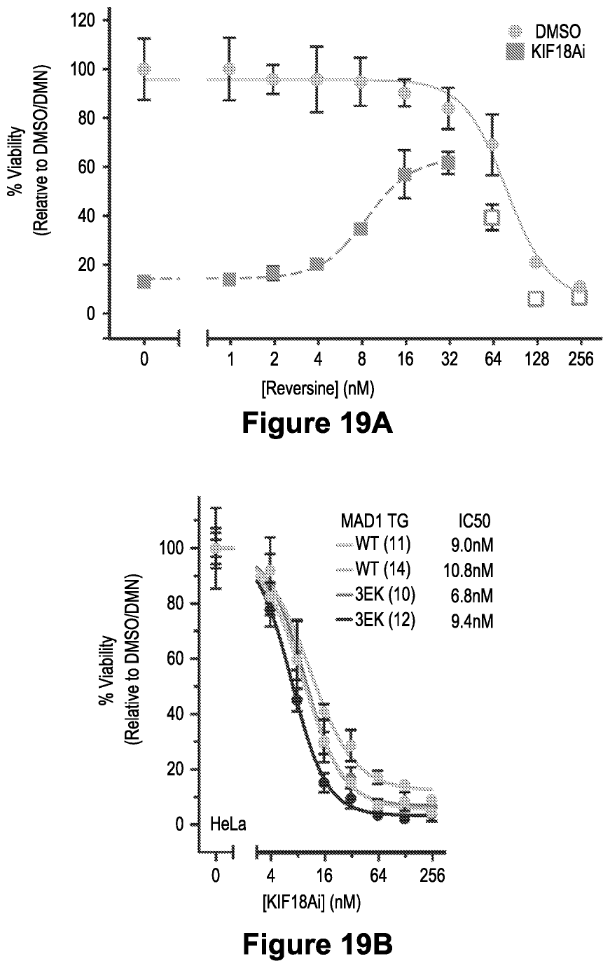

We next sought to directly interrogate the role of the SAC on KIF18Ai toxicity. To test whether SAC suppression was sufficient to relieve KIF18A dependency, we co-treated the panel of sensitive cell lines with both KIF18Ai and low doses of Reversine. Partial SAC silencing rescued KIF18Ai toxicity across the panel of sensitive cell lines (FIG. 9e), with the extent of growth rescue likely limited by the intrinsic sensitivity to SAC inhibition following Reversine treatment (FIG. 19a). We conclude that KIF18Ai toxicity depends on SAC activation to induce mitotic delays.

Since cells with depleted Cyclin B1 or MAD1 were more prone to mitotic slippage, we speculated that they may have a reduced capacity to signal through the SAC. Several SAC proteins including BUBR1 and MAD1 are recruited to unattached kinetochores to execute SAC signaling and are displaced during SAC silencing³⁰⁻³². We therefore employed fixed immunofluorescence to interrogate the degree of SAC signaling with KIF18Ai treatment relative to SAC off (metaphase, vehicle-treated) and SAC on (Nocodazole-treated) conditions. Unexpectedly, rather than resulting in strong SAC signaling at a few problematic kinetochores, KIF18Ai caused a minor increase in BUBR1 and MAD1 intensity across nearly all kinetochores (FIG. 9f-h). Clonal Cyclin B1, MAD1 and HSET edited lines all exhibited a similar capacity to recruit BUBR1 to kinetochores in response to KIF18Ai, suggesting that these cells remained proficient in sensing the KIF18Ai-driven defect (FIG. 9f). By contrast, we found divergent responses with recruitment of the downstream SAC effector MAD1. As expected, MAD1 signal was greatly diminished in MAD1 hypomorphic cells across all treatment conditions. However, we also observed slightly diminished MAD1 signal in Cyclin B1, but not HSET knockout cells (FIG. 9g). We conclude that depletion of Cyclin B1 or MAD1 alleviates KIF18Ai toxicity by reducing the cells' ability to signal through the SAC.

We speculated that elevated SAC signal with KIF18Ai may be the result of KIF18A acting directly in SAC silencing. Displacement of SAC proteins at the kinetochore is driven by phosphatases PP2A-B56 and PP1^{33,34}, and KIF18A harbors a regulatory PP1 binding motif that has been proposed to serve as a kinetochore recruitment platform for the phosphatase^{35,36}. Using the drug-resistant KIF18A transgene system in HCC1806 cells, we generated a KIF18A variant lacking the PP1 binding motif³⁵. As anticipated, WT G289I KIF18A, but not a similar transgene lacking motor activity¹⁴, relieved drug toxicity. However, a G289I KIF18A mutant unable to bind PP1 largely rescued growth (FIG. 9h and FIG. 16b). We conclude that PP1 recruitment to the kinetochore by KIF18A does not play a major role in SAC silencing or KIF18Ai toxicity.

Since Cyclin B1 knockout cells only exhibited a minor SAC signaling defect compared to their potent rescue of KIF18A toxicity, we wondered whether Cyclin B1 might be rescuing viability through other means. Notably, Cyclin B1 is critical for the activation of the master mitotic kinase CDK1 whose activity defines the mitotic state^{37,38}. Accordingly, sub-saturating concentrations of the CDK1 inhibitor RO-3306 provided a robust rescue of viability in KIF18Ai-treated HCC1806 cells, suggesting high Cyclin B1/CDK1 activity is needed to maintain a mitotic arrest and promote KIF18Ai toxicity (FIG. 9j). Furthermore, Cyclin B1 serves a CDK1-independent scaffolding role for MAD1 at the kinetochore corona³⁹, a proteinaceous matrix that expands off of chronically unattached kinetochores to bolster microtubule capture and SAC signaling⁴⁰. However, ablation of the corona pool of MAD1 with targeted mutations in the protein had little impact on KIF18Ai resistance in HeLa cells (FIG. 19b)⁴¹. These findings agree with the comparatively weak rescue of KIF18Ai sensitivity observed with sgRNAs targeting Rod, a component required for corona integrity⁴² (FIG. 9c). This demonstrates that Cyclin B1-mediated activation of CDK1 but not MAD1 recruitment to the corona is required for KIF18Ai toxicity.

### Kif18Ai Toxicity is Relieved by Stabilizing Kinetochore-Microtubule Attachments

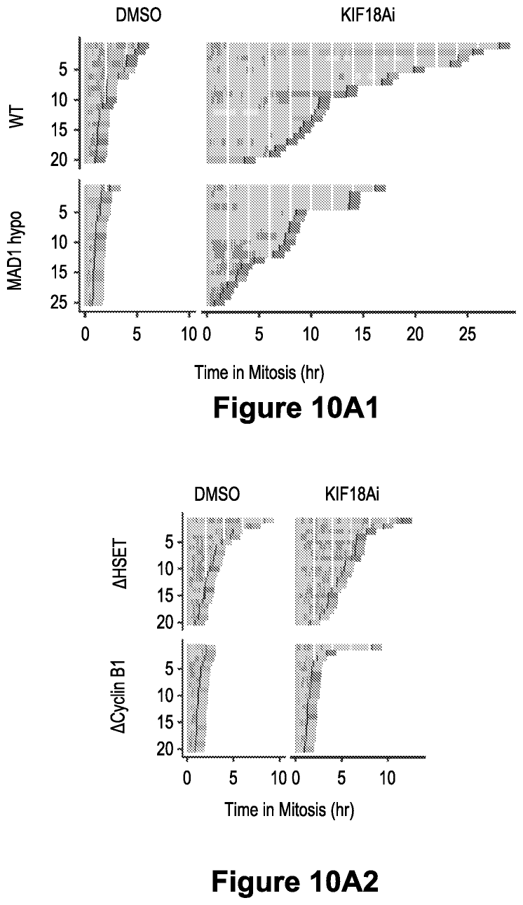

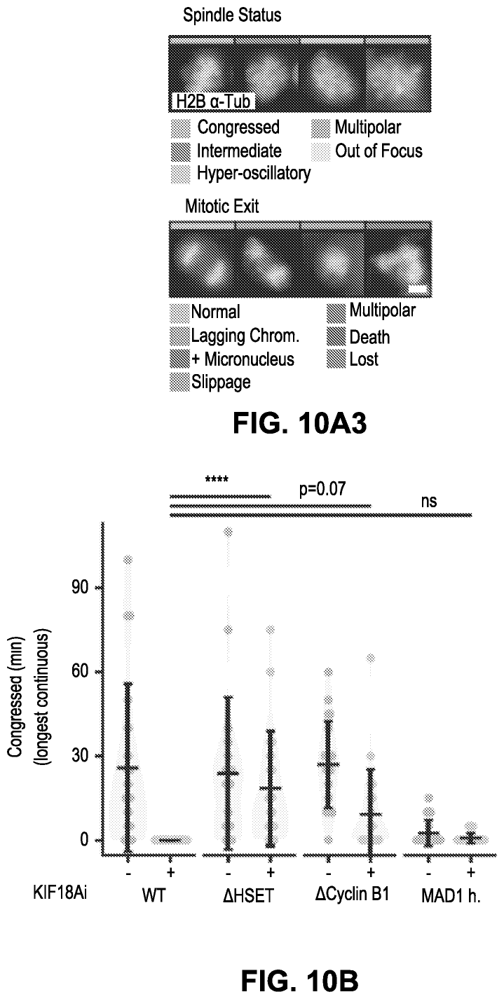

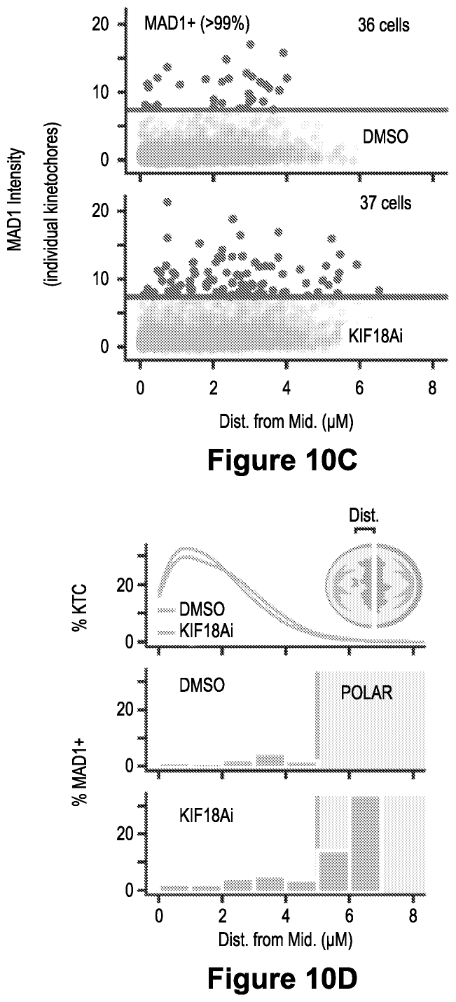

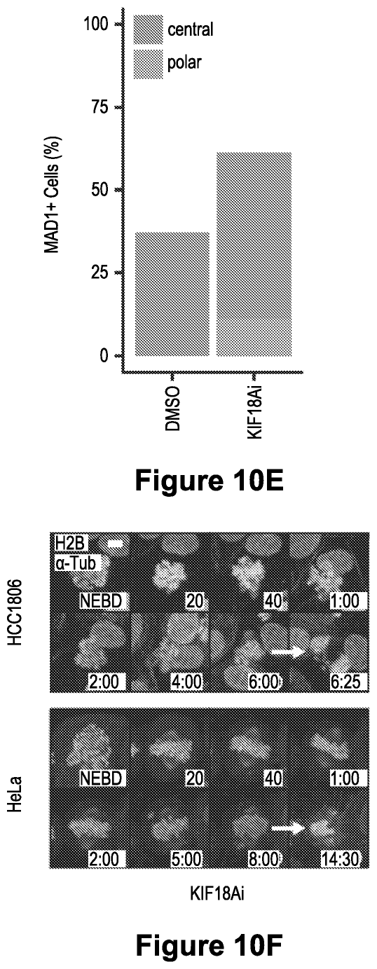

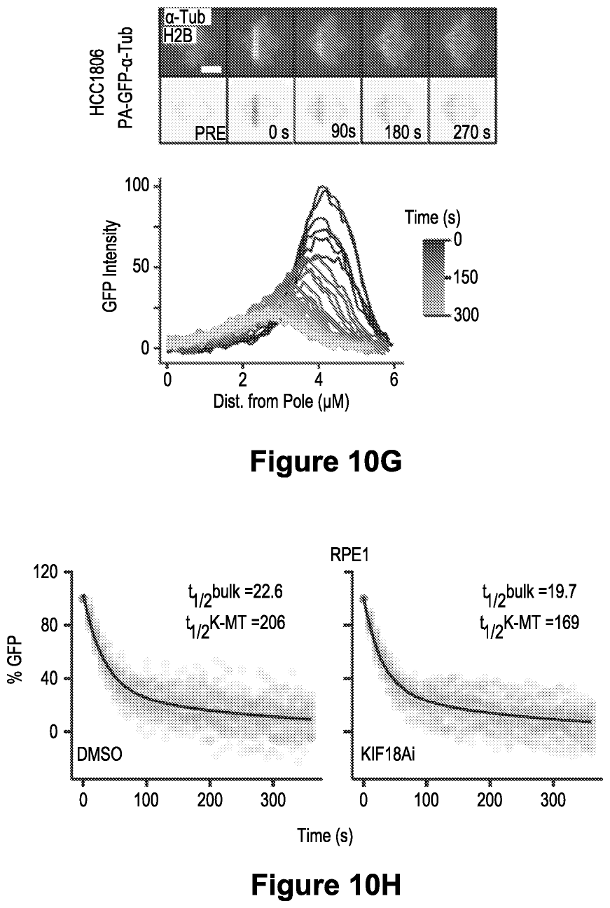

The identification of the minus-end directed kinesin HSET as a KIF18Ai resistance gene led us to speculate that loss of the protein may indirectly silence the SAC by rescuing mitotic spindle dynamics. HSET cross-links and laterally slides microtubule bundles to control mitotic spindle length^{43,44}, and while HSET loss leads to spindle shortening, KIF18A inhibition increases spindle length and causes metaphase plate congression defects. Live-cell timelapse microscopy revealed that KIF18Ai treatment led to a dramatic increase in hyper-oscillatory chromosomes in HCC1806 cells, resulting in a longer mitosis with more errors (FIG. 10a). Knockout of HSET and, to a lesser extent Cyclin B1, dramatically improved the ability to form and maintain a metaphase plate in KIF18Ai (FIG. 10b). By contrast, MAD1 hypomorphs did not rescue chromosome alignment in KIF18Ai. We conclude that loss of HSET and Cyclin B1 counteract the effect of KIF18Ai treatment by dampening chromosome alignment defects.

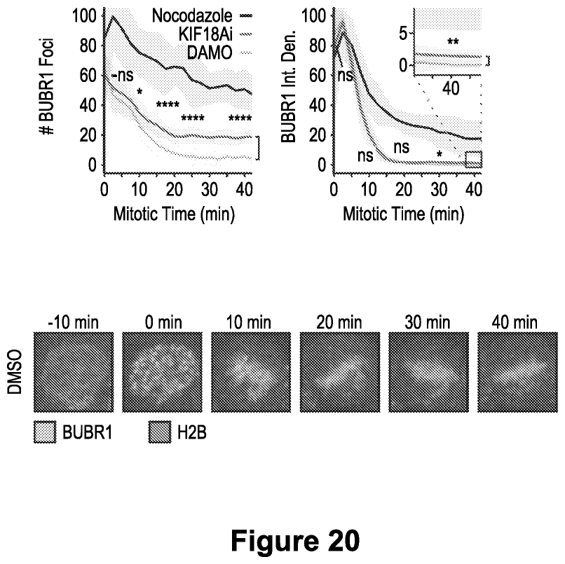

The dramatic chromosome misalignment phenotype in KIF18Ai led us to speculate that hyper-oscillatory chromosomes play a dominant role in SAC signaling and the resulting mitotic arrest. Fixed immunofluorescence analysis of KIF18Ai treated HCC1806 cells revealed that, while kinetochores near the spindle pole were more likely to be SAC active, the majority of SAC-signaling (MAD1+) kinetochores remained within the central chromosome mass (FIG. 10c,d). Furthermore, SAC signaling polar kinetochores were present in fewer than 15% of cells, demonstrating that they are not required for mitotic delays (FIG. 10e). Consistently, Hela cells treated with KIF18Ai experienced a protracted metaphase arrest without chromosome hyper-oscillation (FIG. 10f). Live-cell visualization of SAC signaling with a BUBR1-EGFP reporter also revealed that KIF18Ai treatment increased BUBR1 recruitment to congressed kinetochores in Hela cells (FIG. 20a). We conclude that SAC signaling following KIF18A inhibition does not require chromosome hyper-oscillation.

Changes in kinetochore-microtubule dynamics and attachment stability in response to KIF18Ai treatment could lie upstream of both congression defects and increased SAC signaling^{45,46}. To monitor the stability of kinetochore-microtubule attachments in cells, we used an established live cell timelapse imaging assay with cells expressing a photoactivatable-GFP-α-Tubulin (PA-GFP-a-Tub) transgene⁴⁷ (FIG. 10g). After photo-activation of a spindle region adjacent to the metaphase plate, a second-order decay curve was fit to the loss of PA-GFP-α-Tubulin signal over time. This decay curve has two half-lives: the faster half-life reports on general microtubule turnover (bulk), and the slower half-life reports on the turnover of comparatively stable K-fibers (K-MT)⁴⁸. Delays in the slower half-life represent more stable K-MT attachments.

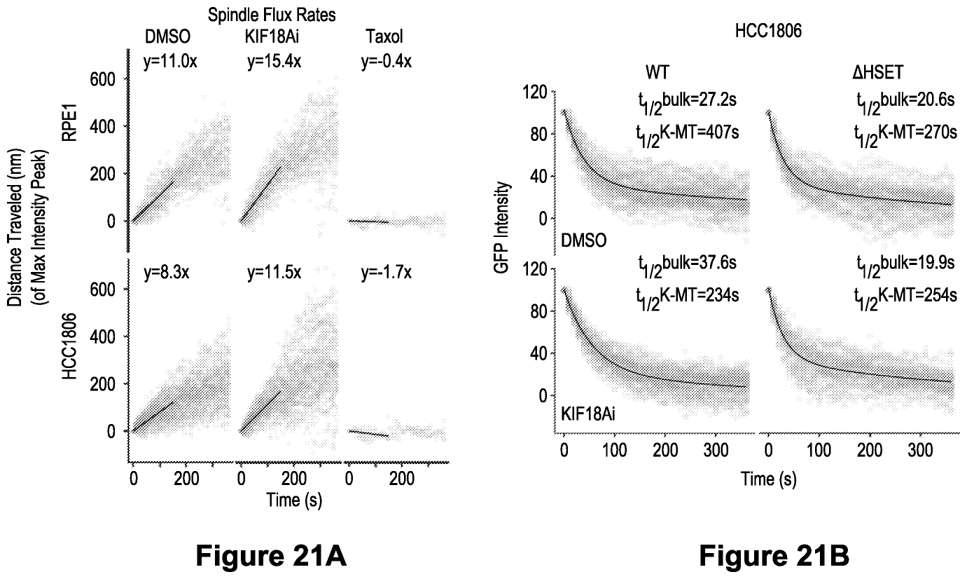

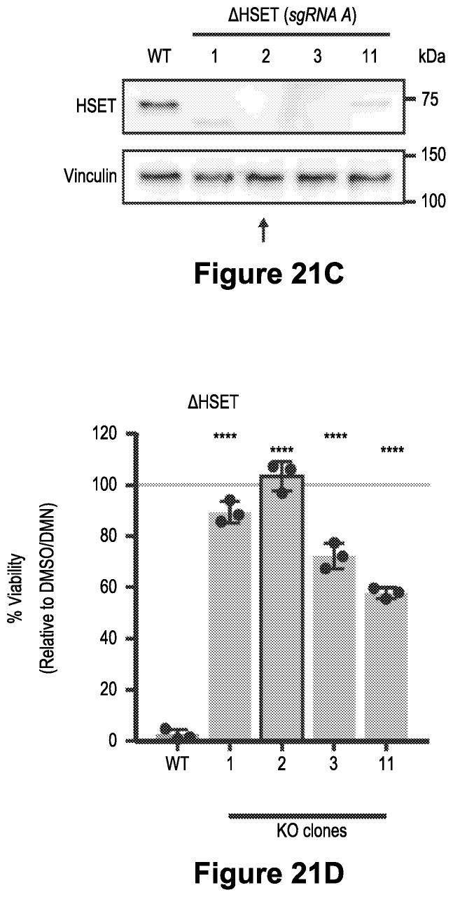

KIF18Ai treatment decreased the K-MT half-life by 18% in insensitive RPE1 cells, indicating a slight destabilization of attachments (FIG. 10h,i). This was accompanied by an increase in the rate of microtubule poleward flux, as previously observed in Drosophila melanogaster cells⁴⁹ (FIG. 21a). CIN cancers have been shown to exhibit more stable K-MT attachments⁵⁰, and consistently, a 3-fold increase K-MT stability was seen in untreated HCC1806 cells relative to RPE1 cells (FIG. 10i and FIG. 21a,b). Interestingly, KIF18Ai treatment caused a 33% reduction in K-MT attachment stability in these cells, suggesting that sensitive cells may be reliant on KIF18A for K-MT attachment hyperstability. Importantly, knockout of HSET partially corrected baseline stability and mitigated the destabilization of K-MT attachments caused by KIF18Ai treatment, likely explaining the alleviated KIF18Ai toxicity in these cells. We conclude that KIF18A inhibition strongly destabilizes hyper-stable K-MT attachments in sensitive HCC1806 cells and that HSET loss partially corrects this defect.

### Kif18Ai-Driven SAC Signaling Occurs in Both Sensitive and Insensitive Cell Lines

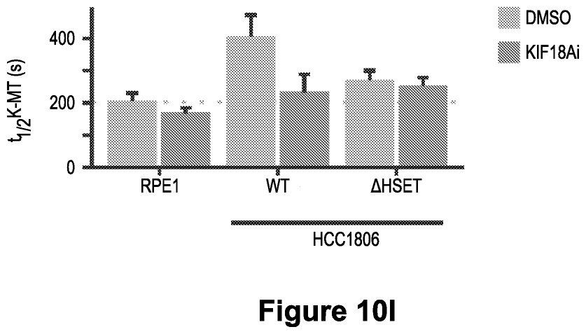

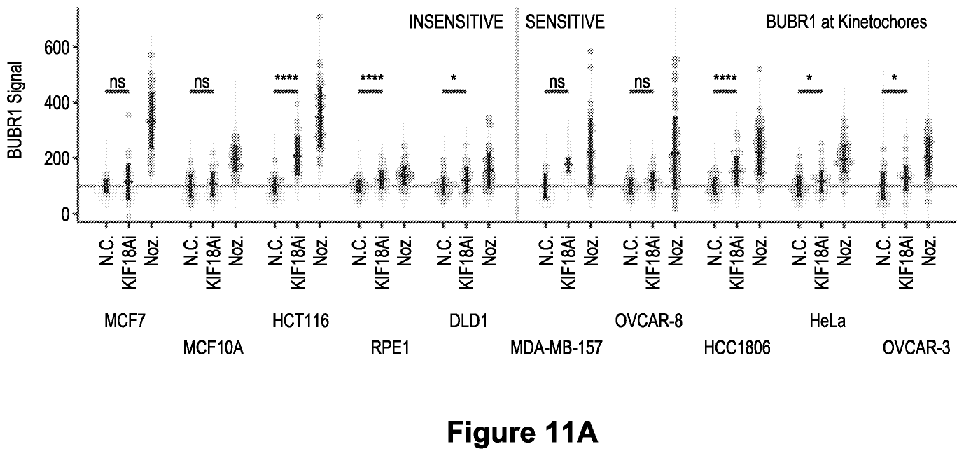

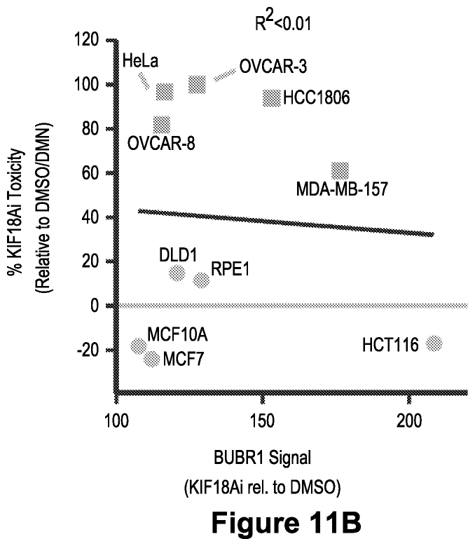

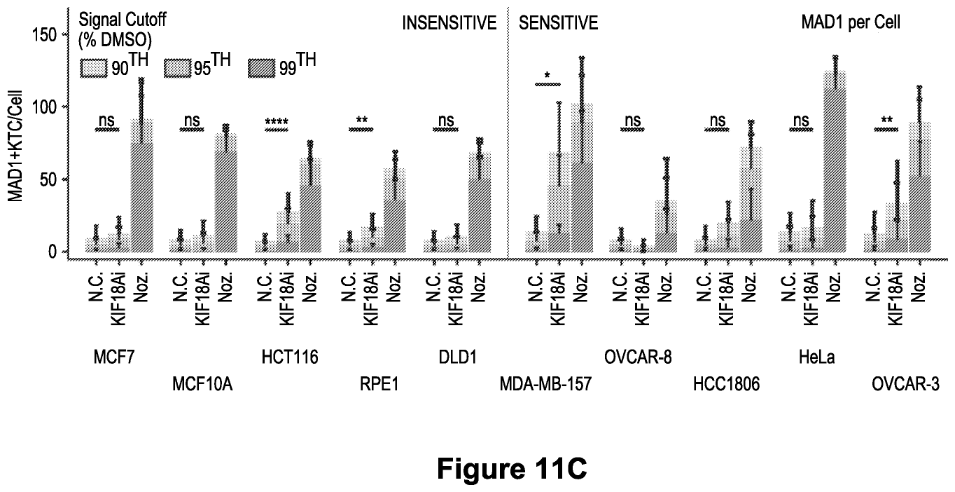

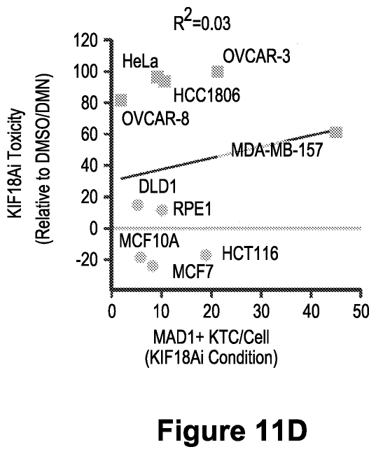

We hypothesized that increased reliance on KIF18A for K-MT attachment stability would translate into stronger SAC signaling after KIF18Ai treatment. We therefore extended our fixed immunofluorescence analysis of SAC signaling with KIF18Ai to the entire panel of sensitive and insensitive cell lines. In accordance with a greater disruption of K-MT attachments, KIF18Ai-treated HCC1806 cells recruited more BUBR1 to kinetochores of compared to RPE1 cells (FIG. 11a,b). However, this differential effect was not maintained in the number of MAD1+kinetochores per cell (FIG. 11c,d). Unexpectedly, we also found that both sensitive and insensitive cell lines experienced similar increases in the per-kinetochore BUBR1 signal and the number of MAD1+kinetochores per cell in response to KIF18Ai (FIG. 11a-d). Ultimately, this reveals that SAC signaling at kinetochores is a poor overall predictor of KIF18Ai sensitivity (FIG. 11b,d).

Taken together, our data show that treatment with KIF18Ai leads to a weakening of K-MT attachments and an increased probability of activating the SAC at all kinetochores, thereby delaying mitotic exit. Ultimately the mitotic delay, and by extension KIF18Ai toxicity, can be attenuated by reducing the ability to maintain the mitotic state through SAC disruption (MAD1 hypomorph, Cyclin B1 knockout), reduction of CDK1 activity (Cyclin B1 knockout), or stabilization of K-MT attachments and the metaphase plate (HSET knockout). Finally, we find that the degree of SAC signaling does not correlate well with sensitivity.

### Hyperploidy Increases KIF18Ai SAC Signaling to Drive Toxicity.

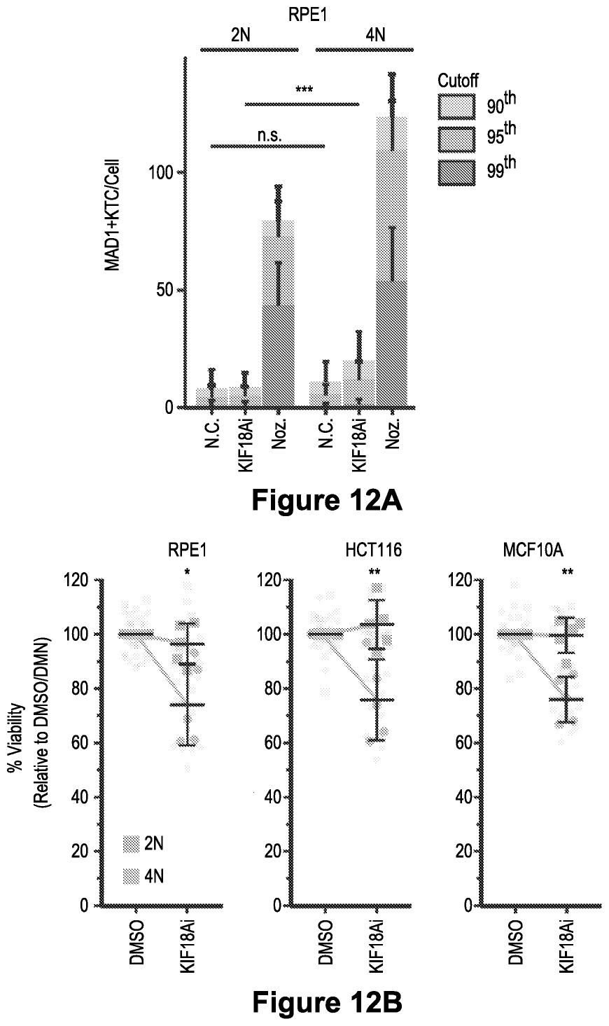

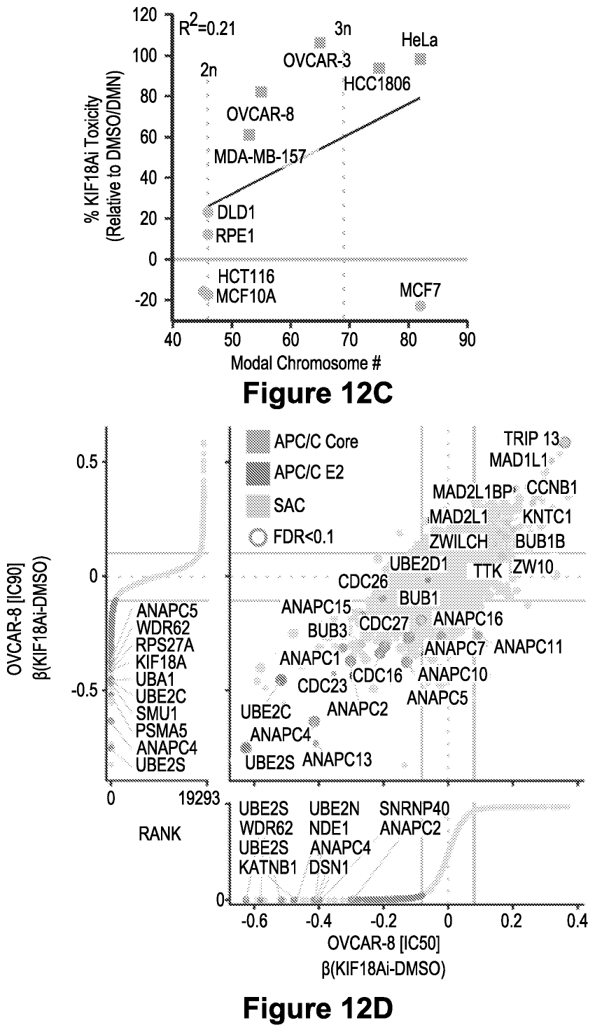

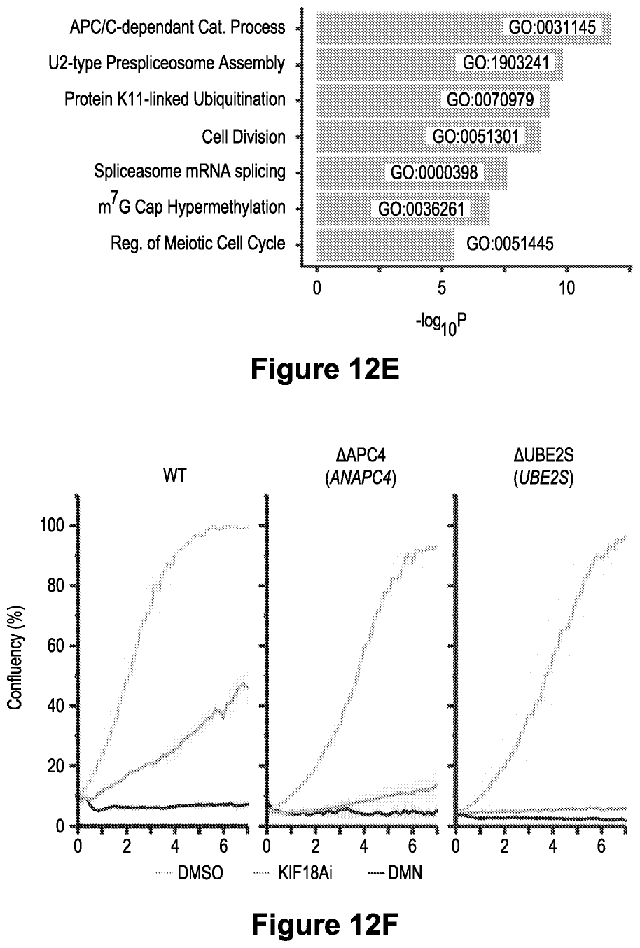

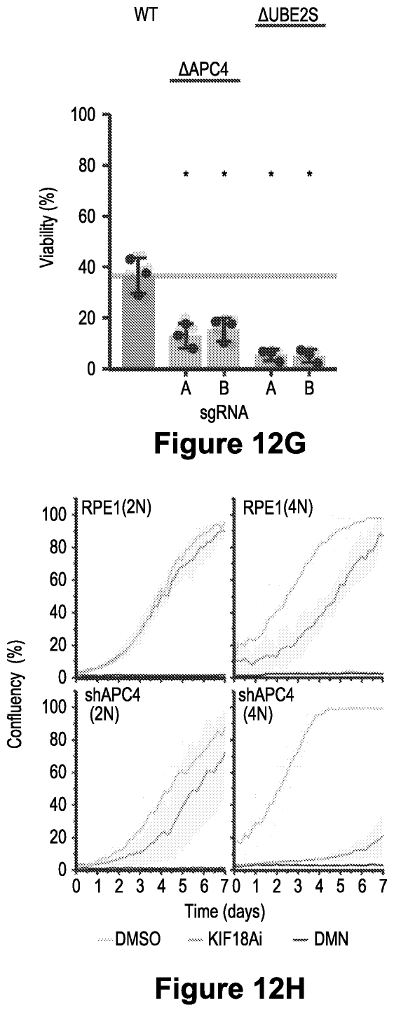

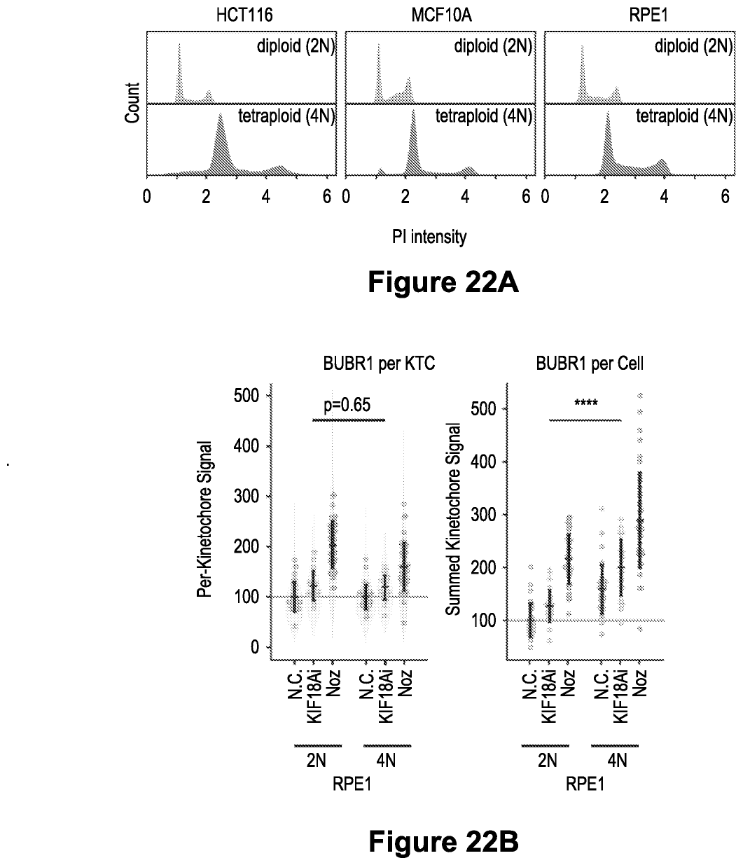

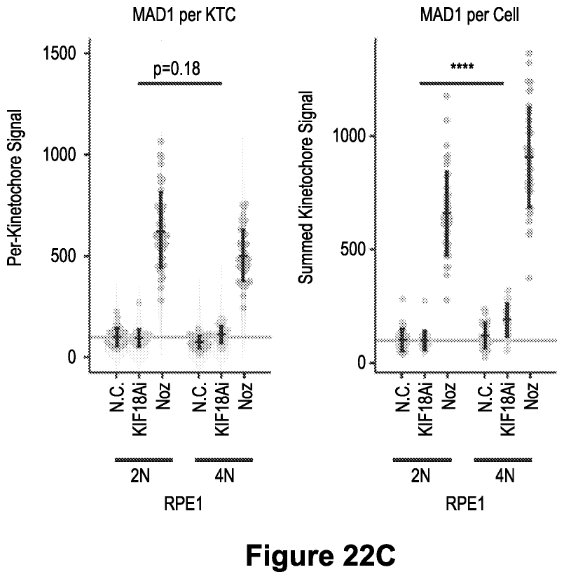

WGD has been shown to increase sensitivity to KIF18A loss¹⁰, and we speculated that higher ploidy might aggravate KIF18Ai toxicity by increasing the number of kinetochores taking part in low-level SAC signaling. To define the impact of chromosome number on KIF18A dependency, we treated matched pairs of diploid and tetraploid RPE1, HCT116 and MCF10A cells with KIF18Ai (FIG. 22a). Immunofluorescence imaging revealed a modest increase in the total number of MAD1+kinetochores in KIF18Ai-treated RPE1 tetraploid cells as compared to diploid controls (FIG. 12a). In addition, while the average per-kinetochore BUBR1 and MAD1 signal was similar or slightly increased between KIF18Ai-treated diploid and tetraploid cells, the cumulative kinetochore signal per cell increased by 56% for BUBR1 and 88% for MAD1 in tetraploid cells treated with KIF18Ai (FIG. 22b,c). Consistently, doubling chromosome number led to a ˜25% increase in toxicity to KIF18Ai in a 5-day viability assay across cell lines (FIG. 12b). However, the KIF18Ai toxicity seen in the tetraploid cells failed to reach the level observed in most sensitive cancer cell lines (FIG. 8c), and chromosome number weakly correlated with KIF18Ai toxicity across the cell line panel (FIG. 12c). We conclude that increases in chromosome number partially sensitize cells to KIF18Ai by increasing the total mitotic SAC burden. However, this determinant alone is insufficient to explain KIF18A dependency in sensitive cell lines.

### Hyperploidy and Low APC/C Activity Act Synergistically to Amplify KIF18Ai Toxicity.

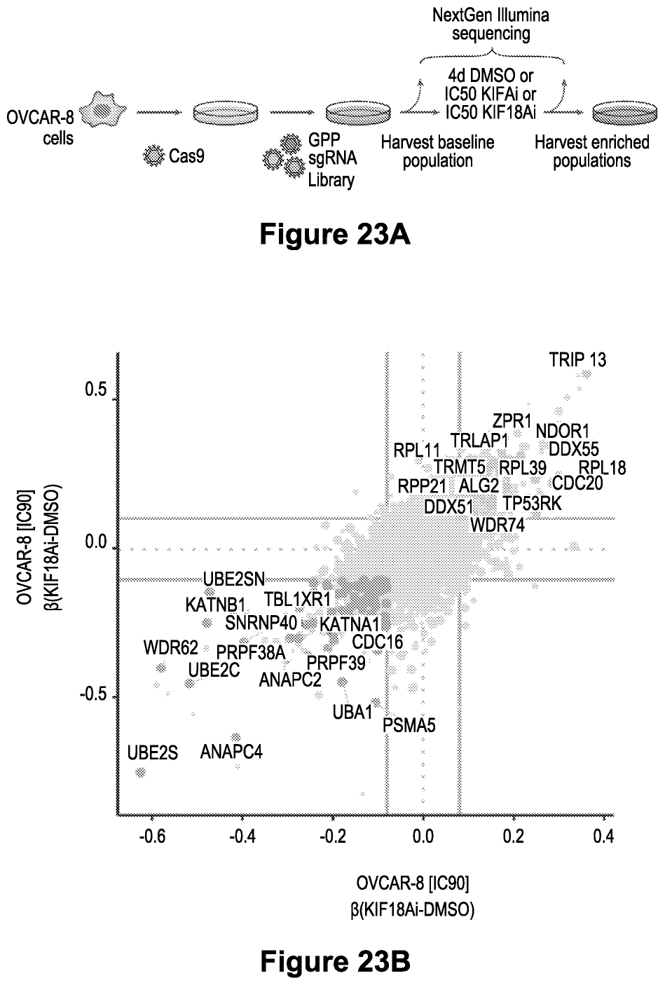

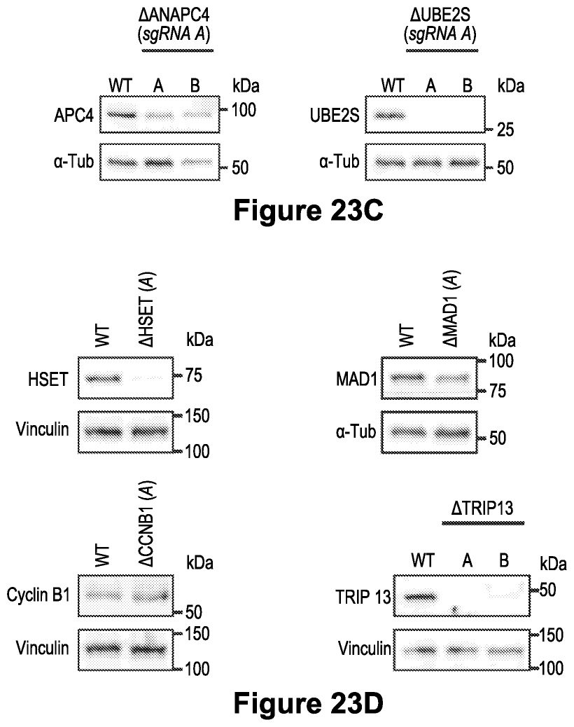

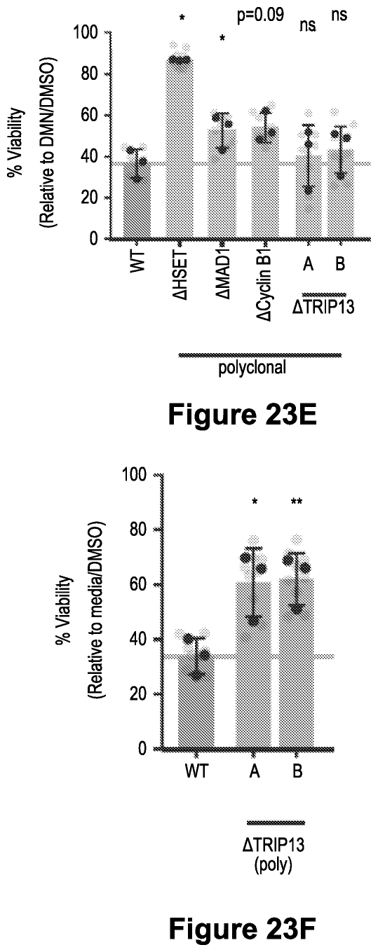

To uncover additional determinants of KIF18A dependency, we ran a genome-wide CRISPR knockout screen in partially sensitive OVCAR-8 cells (82% toxicity; FIG. 8c). We reasoned that the reduced sensitivity of OVCAR-8 cells to KIF18Ai would enable the identification of genes and pathways that increase sensitivity to the drug, in addition to genes promoting resistance. Cas9-expressing OVCAR-8 cells were transduced with a lentiviral knockout library of sgRNAs and selected for 6 days. Cells were then cultured for 4 days in either DMSO or KIF18Ai at IC50 and IC90 concentrations. Guides that altered growth potential in KIF18Ai conditions relative to control were identified by deep sequencing (FIG. 23a). We uncovered a similar set of resistance genes to our previous screens, including MAD1, Cyclin B1, HSET and TRIP13 (FIG. 12d, FIG. 23b-f). By contrast, targeting APC/C subunits or the APC/C E2 ubiquitin ligase UBE2S strongly increased sensitivity to KIF18Ai (FIG. 12d,e). To validate that diminished APC/C activity increased KIF18Ai sensitivity, we used two different sgRNAs to generate polyclonal knockouts of the APC/C subunit APC4 and UBE2S in OVCAR-8 cells (FIG. 23c). Validating the results of the CRISPR screen, KIF18Ai-treated APC4 and UBE2S edited cell lines experienced a 61% and 86% reduction in viability respectively compared to inhibitor-treated WT cells (FIG. 12f,g).

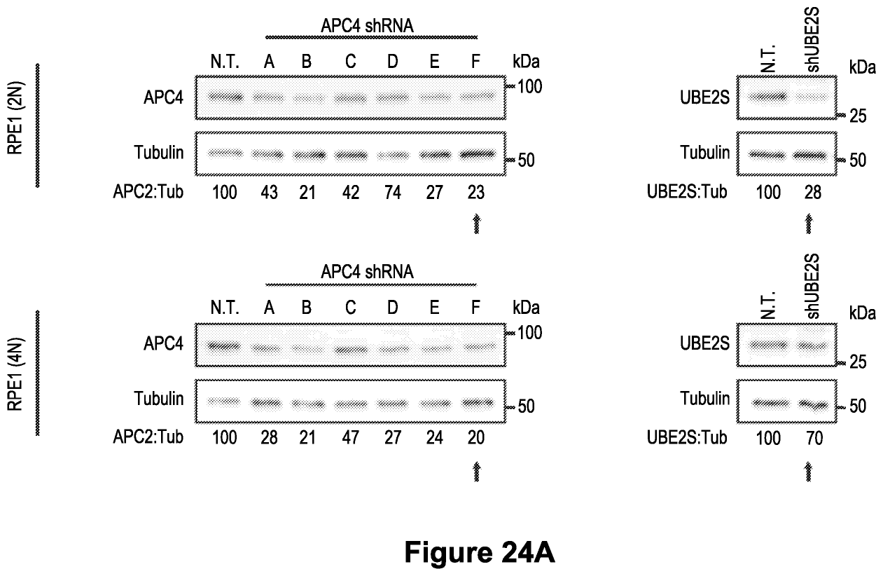

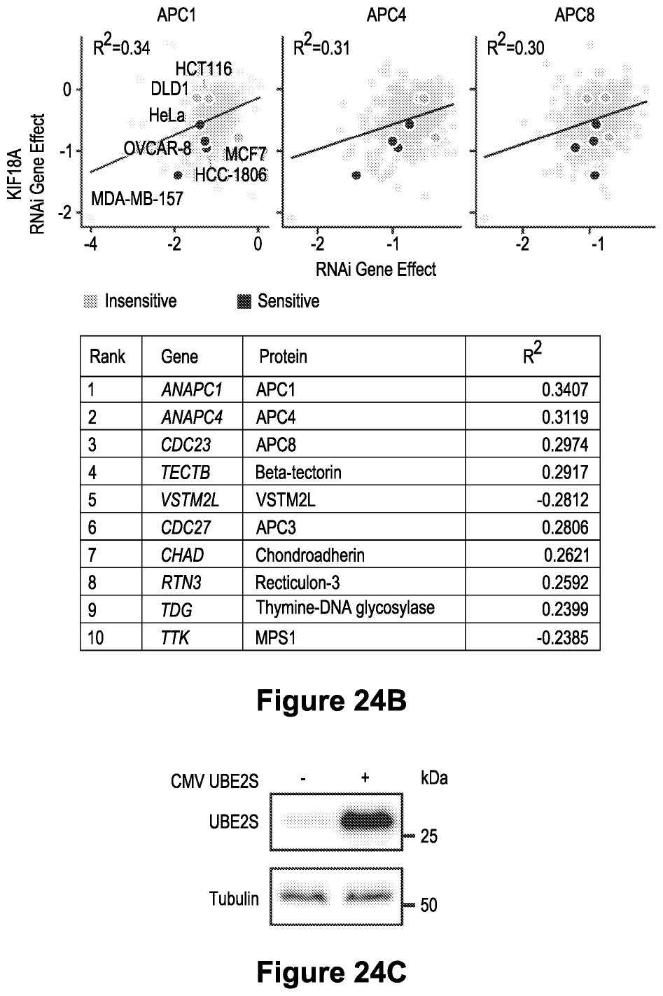

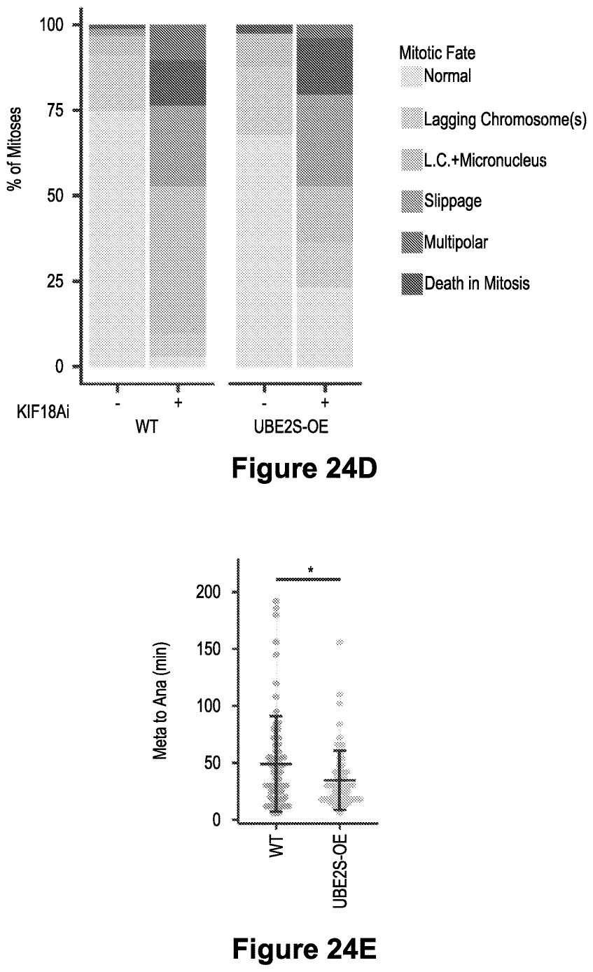

The APC/C is a megadalton-scale E3 ubiquitin ligase responsible for the degradation of Cyclin B1 and Securin to promote the onset of anaphase and timely mitotic exit^{51,52}. Unattached kinetochores generate a SAC signal by catalyzing the assembly of the mitotic checkpoint complex (MCC) that directly binds and inhibits the APC/C to prevent anaphase onset⁵³. By extension, the metaphase-to-anaphase transition is controlled by a sensitive balance between MCC production and basal APC/C activity⁵⁴. We therefore reasoned that KIF18Ai treatment might upset this balance by modestly increasing MCC formation at kinetochores with unstable microtubule attachments. We also predict that conditions that aggravate a SAC: APC/C imbalance in KIF18Ai, such as hyperploidy or APC/C depletion, would act together to increase KIF18Ai sensitivity. To test this hypothesis, we reduced APC/C activity with shRNAs targeting APC4 or the E2 ligase UBE2S in both diploid (2N) and tetraploid (4N) RPE1 cells (FIG. 24a). Doubling chromosome number or reduced APC/C activity modestly slowed the growth rate of these insensitive cells treated with KIF18Ai. By contrast, combining low APC/C activity with increased chromosome number almost entirely blocked proliferation in cells treated with KIF18Ai (FIG. 12h,i). We conclude that hyperploidy and low APC/C activity act synergistically to generate KIF18A dependency.

### KIF18A Dependency in Cancer Relies on a SAC:APC/C Imbalance in Mitosis

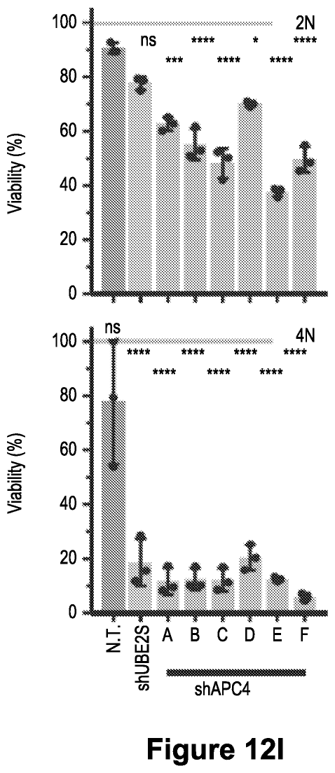

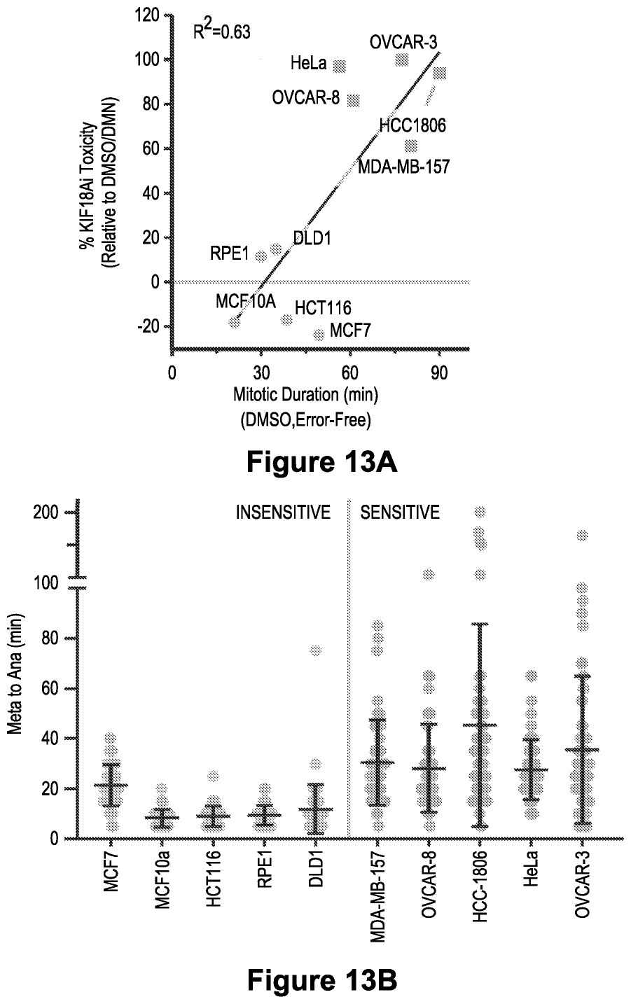

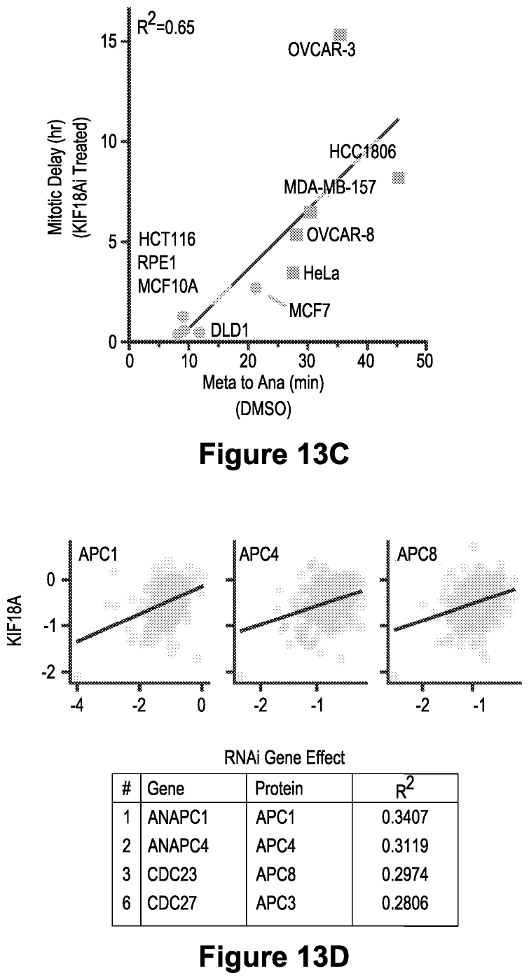

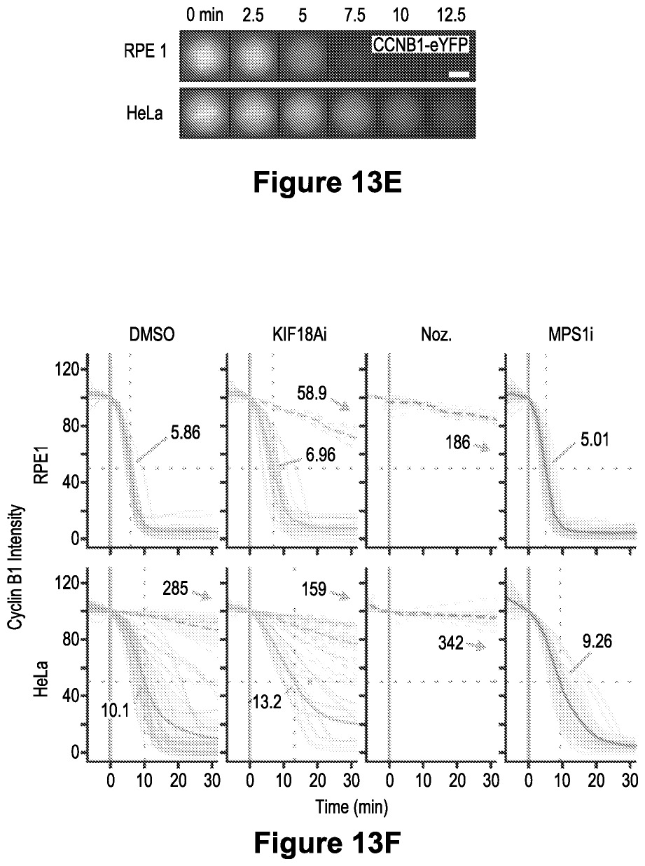

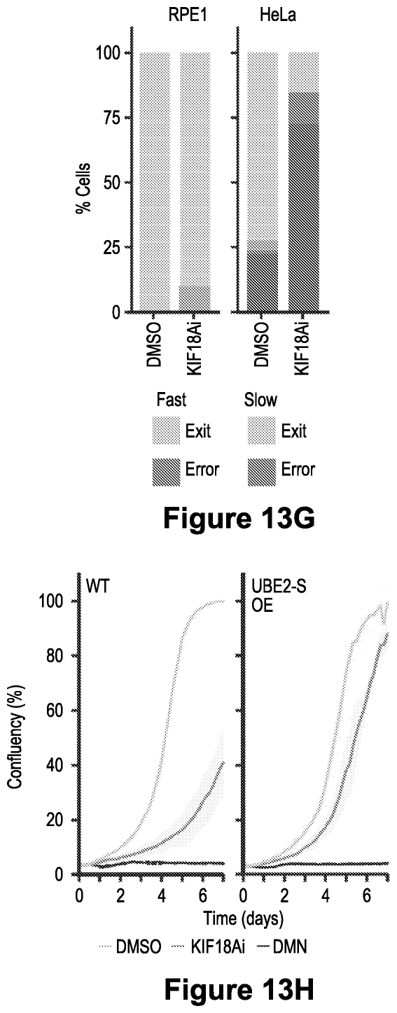

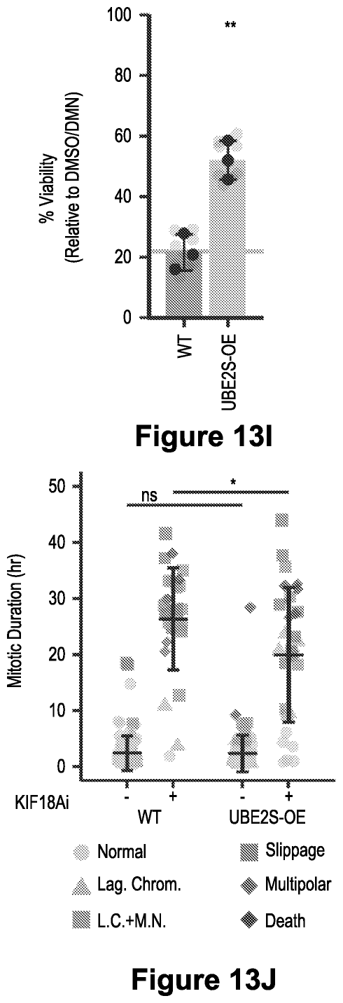

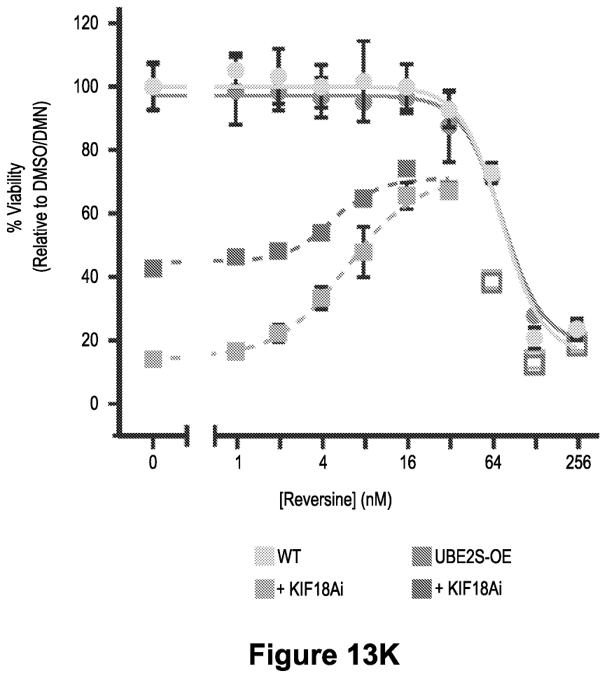

Our data show that disrupting the balance between SAC signaling and APC/C activity is sufficient to generate KIF18A dependency. We therefore determined whether such an imbalance exists in sensitive cell lines. The ratio between SAC signaling and basal APC/C activity is difficult to assay directly, but we reasoned that mitotic delays could serve as signatures for increased SAC signaling, while a slowed metaphase to anaphase transition would indicate low APC/C activity. Indeed, close inspection of earlier live cell timelapse experiments revealed KIF18Ai sensitive cells took more time to complete mitosis in unperturbed conditions (FIG. 13a). Furthermore, mitotic duration proved to be a much stronger correlate for KIF18Ai toxicity (R²=0.63) compared to chromosome number (R²=0.21). Finally, all sensitive cells and insensitive MCF7 cells displayed an extended metaphase-to-anaphase transition that correlated closely with the propensity of these cell lines to exhibit mitotic delays in KIF18Ai (R²=0.65) (FIG. 13b,c).

To examine if the relationship between reduced APC/C activity and KIF18Ai toxicity held true across a broad set of cancer cell lines, we interrogated the Broad Dependency Map (DepMap) database which aggregates CRISPR knockout and RNAi whole genome screening data from over 1000 and 600 cell lines respectively^{55,56}. The DepMap uses these data to generate gene dependency scores that reflect the fitness change after disruption of each gene in each cell line. Co-dependent genes exhibit similar patterns and magnitudes of fitness change across the cell line panel, and a linear regression between cell-specific gene dependency scores is used to assess this relationship. Importantly, using the RNAi dataset which has been shown to be more sensitive for interrogating the effect of depleting essential genes⁵⁷, the top three positively correlated co-dependencies for KIF18A's RNAi were the APC/C subunits APC1, APC4, and APC8 (FIG. 13d and FIG. 24b). This genetic relationship suggests that cell lines sensitive to KIF18A depletion are intolerant of reductions in APC/C activity, likely reflecting an underlying SAC: APC/C imbalance. We therefore conclude that KIF18A dependency and SAC: APC/C imbalance are broadly linked.

To directly assay APC/C activity in cells, we directly monitored degradation of the APC/C substrate Cyclin B1 at the metaphase to anaphase transition. Live-cell experiments showed that endogenously tagged Cyclin B1-YFP was degraded more slowly in KIF18Ai-sensitive Hela cells compared with insensitive RPE1 cells, reflecting reduced APC/C activity (FIG. 13e,f). While Cyclin B1 degradation rates were fast (t_{1/2}<30 min) in all the RPE1 cells examined, in Hela cells we observed two degradation modalities: fast Cyclin B1 degradation after SAC silencing (t_{1/2}<30 min) or slow degradation resulting from partial inhibition of APC/C activity (t_{1/2}>=30 min). This second population likely represents cells with persistent chromosome attachment defects that fail to fully silence the SAC. Treatment with KIF18Ai caused a dramatic shift from fast to slow degradation modalities in KIF18Ai-sensitive Hela cells, while a very modest shift was observed in insensitive RPE1 cells (FIG. 13g). Slow degradation rates of Cyclin B1 also coincided with a high frequency of mitotic failure. Finally, abrogation of the SAC with Reversine resulted in similar degradation rates to untreated cells, suggesting that the slower Cyclin B1 degradation rate in Hela cells truly reflects lower basal APC/C activity. Taken together, these data provide evidence for a SAC: APC/C imbalance in sensitive HeLa cells, which primes these cells for mitotic delay in KIF18Ai.

### Increasing APC/C Activity Rescues KIF18Ai Toxicity in Sensitive Cell Lines

Our data predict that increasing APC/C activity would correct the SAC: APC/C imbalance and reduce KIF18Ai toxicity in sensitive cell lines. To test this, we sought to increase APC/C activity by overexpression of its E2 ligase UBE2S in HCC1806 cells^{58,59} (FIG. 24c). Indeed, overexpression of UBE2S resulted in a ˜40% reduction in KIF18Ai toxicity (FIG. 13h,i), a reduction of KIF18Ai-driven mitotic delays and errors (FIG. 13j and FIG. 24d), and a decrease in metaphase to anaphase duration (FIG. 24e). To examine whether heightened APC/C activity increased the tolerance for SAC signaling in KIF18Ai, we titrated Reversine into WT and UBE2S overexpressing cells treated with KIF18Ai. As before, partial SAC inhibition with low doses of Reversine led to a potent rescue of KIF18Ai toxicity, while higher concentrations were toxic to cells (FIG. 13k). The maximal viability rescue was not additive between UBE2S overexpression and Reversine, suggesting that these rescues are acting via the same pathway. However, maximal rescue was reached at lower concentrations of Reversine in UBE2S overexpressing cells, indicating that increased APC/C activity directly translates into a higher tolerance for the low-level SAC signaling caused by KIF18Ai. In other words, high basal APC/C activity can directly mitigate low levels of SAC signaling to promote anaphase onset.

### DISCUSSION

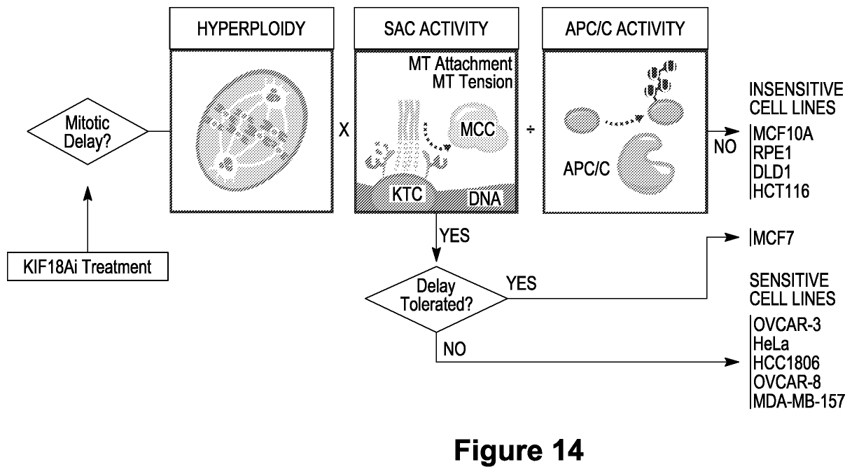

Identifying appropriate patient populations is vital to the clinical success of KIF18A inhibitors. Previous work has identified WGD and CIN as indicators of KIF18A dependency^{10,11}. However, these correlates were only weakly predictive of KIF18Ai sensitivity, suggesting that additional determinants remained to be uncovered. Furthermore, the Broad DepMap cancer database reports that KIF18A dependency is not readily predicted by expression, lineage, or mutational datasets⁶⁰, reinforcing the need for a mechanistic understanding of KIF18A dependency. Here we show that toxicity from KIF18A inhibition is caused by prolonged mitotic delays mediated by persistent SAC signaling. Treatment with KIF18Ai generates a minor increase in SAC signaling across all cell lines, due to the weakening of microtubule-kinetochores attachments. Whether cell lines tolerate this defect and enter anaphase depends on the magnitude of the SAC burden as well as the basal level of APC/C activity. Hyperploidy serves as a multiplicative factor that magnifies the total SAC signal produced at individual kinetochores. Conversely, high basal APC/C activity can mitigate the increase in SAC signal to allow mitotic exit. Taken together, we show that increased SAC signal and low basal APC/C activity play a central role in driving KIF18Ai sensitivity (FIG. 14a).

A unique feature of KIF18A inhibition compared with other antimitotic agents is its ability to apply a gentle pressure toward mitotic delay. Unlike the microtubule-stabilizing agent Taxol, which shows broad cytotoxicity by promoting persistent SAC signaling, KIF18A inhibition generates a small increase in SAC signal through unstable kinetochore-microtubule attachments. In doing so, KIF18Ai selectively targets cells that are already predisposed to mitotic delays while sparing cells that progress through mitosis quickly. Indeed, the best predictor of KIF18Ai sensitivity is the duration of unperturbed mitosis. Consistently, the intermediate increase in SAC signaling with KIF18Ai generates a graded severity of mitotic responses, as with the increased sensitivity observed with hyperploid cell lines that magnify the total SAC burden.

Several lines of evidence underscore a central role of SAC signaling and APC/C activity in mediating KIF18Ai-driven mitotic delays and toxicity. First, decreasing APC/C activity or increasing ploidy produces modest increases in KIF18A sensitivity that synergize strongly. Second, there is a strong correlation between the duration of the metaphase-to-anaphase transition and KIF18Ai toxicity. Third, we show that a reduction of SAC signaling, through genetic or pharmacological means, alleviates KIF18Ai toxicity and may present opportunities for resistance. In this vain, others have shown that knockout of the SAC component MAD2 increases the viability of KIF18A depleted cells¹⁸. Finally, increases in basal APC/C activity provide resistance to KIF18Ai. Thus, increased SAC signal or decreased APC/C activity predispose cells to metaphase delays and KIF18Ai sensitivity.

A better appreciation of the role of the APC/C in generating KIF18Ai sensitivity raises the question of why cancer cells would select for APC/C dysfunction. A recent report showed that lowered APC/C activity plays a protective role by tempering excessive CIN⁶¹. In these cells, the benefits of tumor heterogeneity are balanced against the fitness cost of genome instability to maximize tumor growth potential. This may help explain why CIN cancers have been shown to be, on average, more sensitive to KIF18A loss¹¹. Additionally, reduced APC/C activity is a known resistance mechanism to SAC-targeting antimitotic agents such as MPS1 inhibitors⁶². Typically, SAC ablation results in premature and catastrophic mitotic exit, however, lowered APC/C activity slows the rate of Cyclin B1 and Securin degradation, granting additional time for correct chromosome alignment before anaphase onset. We therefore speculate that KIF18A inhibitors might serve as a second-line treatment for tumors that have developed resistance to SAC inhibitors.

Although our data provide functional evidence for a SAC: APC/C imbalance in KIF18Ai sensitive cell lines, dissecting the underlying cause of this imbalance has proven challenging. For instance, SAC signaling strength can be altered by ploidy, K-MT attachment quality, the abundance and recruitment of SAC signaling proteins, the abundance and recruitment of SAC-silencing phosphatases, or the efficiency of MCC recycling factors to name a few. Similarly, APC/C function may be limited by the expression of any one of its 13 subunits, abundance and activity of E2 conjugating enzymes, or recruitment of its mitotic coactivator CDC20. The combination of alterations that generate the same phenotypic outcome could differ from one cancer to the next. Perhaps because of this, we have been unable to identify specific mutations or expression pattern changes that can predict KIF18A dependency with high confidence. On the contrary, we show that basal mitotic duration correlates well with KIF18Ai toxicity because it wholistically reports on the SAC: APC/C imbalance in a way that is agnostic of the underlying dysfunction. However, although mitotic duration can be established experimentally in cell lines, it cannot be directly assayed in patient tumors. This highlights an opportunity to identify better biomarkers that can accurately report on this imbalance, and by extension, KIF18A dependency.

Potent KIF18Ai toxicity is generated when cells suffer a protracted mitotic delay and lethal errors in cell division. However, a surprising feature of KIF18Ai toxicity was the variation in response to mitotic delay. For example, while Hela cells treated with KIF18Ai had highly stable metaphase plates that eventually underwent cohesion fatigue, HCC1806 cells struggled to form and maintain a congressed metaphase. By contrast, insensitive MCF7 cells had robust mitotic spindles that could delay in mitosis up to seven hours and ultimately divide normally. This suggests that spindle robustness plays an important role in determining sensitivity to KIF18Ai and possibly other drugs that induce mitotic delays. Understanding this cellular response to mitotic delays is an underappreciated feature of antimitotic therapies and an opportunity for further research.

### Example 9

This example describes the materials and methods used in Example 8.

No statistical methods were used to predetermine sample size. The experiments were not randomized and investigators were not blinded to allocation during experiments and outcome assessment.

### Cell Lines and Culture Conditions

HeLa, HCT116, and DLD1 cells were grown in DMEM medium (Corning Cellgro) containing 10% fetal bovine serum (Sigma), 100 U/mL penicillin, 100 U/mL streptomycin and 2 mM L-glutamine. hTERT RPE-1 cells were grown in DMEM: F12 medium (Corning Cellgro) containing 10% fetal bovine serum (Sigma), 0.348% sodium bicarbonate, 100 U/mL penicillin, 100 U/mL streptomycin and 2 mM L-glutamine. MDA-MB-157, OVCAR-8, HCC1806, and MCF7 cells were grown in RPMI 1640 medium (ThermoFisher Scientific) containing 10% fetal bovine serum (Sigma), 100 U/mL penicillin, 100 U/mL streptomycin and 2 mM L-glutamine. OVCAR-3 cells were grown in RPMI 1640 medium (ThermoFisher Scientific) containing 20% fetal bovine serum (Sigma), 100 U/mL penicillin, 100 U/mL streptomycin, 2 mM L-glutamine, and 10 μg/ml bovine insulin (Sigma). MCF10A cells were grown in DMEM: F12 medium (Corning Cellgro) containing 5% horse serum (Invitrogen), 100 U/ml penicillin, 100 U/ml streptomycin, 20 ng/ml hEGF, 0.5 μg/mL hydrocortisone (Sigma), 50 ng/ml cholera toxin (Sigma) and 10 μg/ml bovine insulin (Sigma). All cell lines were maintained at 37° C. in a 5% CO2 atmosphere with 21% oxygen and routinely checked for mycoplasma contamination.

HeLa MAD1 knockout FRT TetON VSV-MAD1 (WT), HeLa MAD1 knockout FRT TetON VSV-MAD1 (E52K, E53K, E56K), and RPE1 Cyclin B1-EYFP cells were a kind gift of A. Saurin (Uni. If Dundee). RPE1 PA-GFP-α-Tubulin cells were a kind gift of D. Compton (Dartmouth College). Paired genome doubled cell lines RPE1 (2N) and (4N), HCT116 (2N) and (4N), and MCF10A (2N) and (4N) cells were a kind gift of N. Ganem (Boston University).

### Gene Targeting and Stable Cell Lines

To generate fluorescent histone H2B and α-Tubulin-labelled cell lines, ORFs were cloned into FUGW lentiviral vectors. Fluorescent populations of cells were then generated by lentivirus-mediated transduction. DLD1 cells were transduced with H2B-mRFP and YFP-α-Tubulin. HCT116 and MCF10A cells were transduced with H₂B-iRFP. HCC1806, MCF7, MDA-MB-157, RPE1, and OVCAR-8 cells were transduced with H2B-iRFP and eGFP-α-Tubulin. Hela cells were transduced with H2B-mRFP and eGFP-α-Tubulin. OVCAR-3 cells were transduced with H2B-eGFP. Polyclonal populations of cells expressing the desired fluorescent markers were used directly or isolated using FACS.

To generate CRISPR-Cas9-mediated knockout lines, sgRNAs targeting APC4 (ANAPC4-A, 5′-aacatgtatgtgtgaagcat-3′ (SEQ ID NO: 35); ANAPC4-B, 5′-gtcacagaagtctctaccaa-3′ (SEQ ID NO: 36)), Cyclin B1 (CCNB1-A, 5′-gtcagaccaaaatacctact-3′ (SEQ ID NO: 37); CCNB1-B, 5′-gaggccaagaacagctcttg-3′ (SEQ ID NO: 38)), EB1 (MAPRE1-A, 5′-tggaaaagactatgaccctg-3′ (SEQ ID NO: 39); MAPRE1-B, 5′-ctcaacacagagaaccgctg-3′ (SEQ ID NO: 40)), HSET (KIFC1-A, 5′-actggaggggcatttagcca-3′ (SEQ ID NO: 41); KIFC1-B, 5′-gcatactggatagccatcca-3′ (SEQ ID NO: 42)), MAD1 (MAD1L1-A, 5′-gaagaagcgcgagacccacg-3′ (SEQ ID NO: 43); MAD1L1-B, 5′-gctggacctgcaacacaagt-3′ (SEQ ID NO: 44)), Rod (KNTC1-A, 5′-aagctaacgatgaaaatcgg-3′ (SEQ ID NO: 45); KNTC1-B, 5′-aaacattcggaacactatgg-3′ (SEQ ID NO: 46)), TRIP13 (TRIP13-A, 5′-cgagtcgccaacggtccacg-3′ (SEQ ID NO: 47); TRIP13-B, 5′-ttgtgtttggtgattacaca-3′ (SEQ ID NO: 48)), and UBE2S (UBE2S-A, 5′-aactcaccagcagtacgtgt-3′ (SEQ ID NO: 49); UBE2S-B, 5′-catcaaggtctttcccaacg-3′ (SEQ ID NO: 50)) were cloned into either LentiCRISPR v2 puro or LentiCRISPR v2 blast vectors (#52961, #83480; Addgene). Knockout cells were then generated by lentivirus-mediated transduction of these constructs. Positive selection of transduced cells was performed 2 days after transfection with 1 μg/mL puromycin or 5 μg/mL blasticidin, respectively. Monoclonal cell lines were isolated by limiting dilution. The ablation of protein production was assessed by immunoblotting.

To generate the HCC1806 H2B-iRFP photoactivatable (PA)-GFP-α-Tubulin cell line, H2B-iRFP was cloned into a FUGW lentiviral vector and introduced to cells by lentivirus-mediated transduction. Fluorescent cells were isolated using FACS. PA-GFP-α-Tubulin was then cloned into a modified FUGW lentiviral vector with a puromycin-resistance cassette and introduced to cells by lentivirus-mediated transduction. Monoclonal cell lines were isolated by limiting dilution and PA-GFP-α-Tubulin expression was evaluated using fluorescence microscopy.

To generate overexpression HCC1806 KIF18A mutant cell lines, the following ORFs were cloned into a modified FUGW lentiviral vector with a puromycin-resistance cassette: KIF18A-(3×) HA, WT; KIF18A (G289I )-(3×) HA, drug resistant; KIF18A (G289I , R308A, K311A)-(3×) HA, drug resistant motor dead; KIF18A (G289I , V614A, W617A)-(3×) HA, drug resistant PP1 binding mutant. Overexpression cells were then generated by lentivirus-mediated transduction of these constructs. Positive selection of transduced cells was performed 2 days after transfection with 1 μg/ml puromycin. Expression levels of the transgenes were assessed by immunoblotting.

To generate HeLa endogenously-tagged CCNB1-EYFP (Cyclin B1) cells, an sgRNA targeting the Cyclin B1 translational stop codon (5′-gtgtaacttgtaaacttgagt-3′) was cloned into a pX459 vector (#62988; Addgene). A plasmid vector containing >950 bp gene homology arms and an EYFP tag was used as a repair template (kind gift of A. Saurin). Hela cells were transiently transfected (X-tremeGENE HP, Roche) with the pX459 plasmid and repair vector. Fluorescent cells were isolated 6 days after transfection by FACS. Due to low efficiency, a second round of FACS was run to further isolate fluorescent cells 2 weeks later.

To generate HCC1806 H2B-iRFP, EGFP-α-Tubulin, UBE2S overexpression cell lines, the UBE2S ORF was cloned into a modified FUGW lentiviral vector with a puromycin-resistance cassette and introduced to H2B-and-α-Tubulin tagged cells by lentivirus-mediated transduction. Positive selection of transduced cells was performed 2 days after transfection with 1 μg/mL puromycin. UBE2S expression levels were assessed by immunoblotting.

### RNA Interference

Dharmacon pGIPZ lentiviral vectors containing shRNAs targeting UBE2S (5′-acaaatccaggtcccagtg-3′ (SEQ ID NO: 51)), or APC4 (ANAPC4-A, 5′-tatctctggagctaaagcg-3′ (SEQ ID NO: 52); ANAPC4-B, 5′-tatgagtaaactttctggc-3′ (SEQ ID NO: 53); ANAPC4-C, 5′-agtccatctcctatgtcct-3′ (SEQ ID NO: 54); ANAPC4-D, 5′-aactgattcatcaagagag-3′ (SEQ ID NO: 55); ANAPC4-E, 5′-tacaatggaatacagattg-3′ (SEQ ID NO: 56); ANAPC4-F, 5′-tttcctgcacaaacttggt-3′ (SEQ ID NO: 57)) were purchased (Horizon). Stable shRNA-mediated knockdown cell lines were generated by lentivirus-mediated transduction. Positive selection of transduced cells was performed 2 days after transfection with 1 μg/mL puromycin. Knockdown efficiency was assessed by immunoblotting.

### Lentiviral Production and Transduction

Lentiviral expression vectors were cotransfected into HEK 293 FT cells with the lentiviral packaging plasmids psPAX2 and pMD2.G (#12260 and #12259; Addgene). In brief, 3×106 HEK 293 FT cells were seeded into a poly-L-lysine-coated 10-cm culture dish the day before transfection. For each 10-cm dish, the following DNA was diluted in 0.6 ml of OptiMEM (Thermo Fisher Scientific): 4.5 μg of lentiviral vector, 6 μg of psPAX2 and 1.5 μg of pMD2.G. Separately, 35 μL of 1 μg/μl 25 kDa polyethylenimine (PEI; Sigma) was diluted into 600 μL of OptiMEM, briefly vortexed and incubated at room temperature for 5 min. After incubation, the DNA and PEI mixtures were combined, briefly vortexed and incubated at room temperature for 20 min. During this incubation, the culture medium was replaced with 8 ml of pre-warmed DMEM+1% FBS. The transfection mixture was then added drop-wise to the 10-cm dish. Viral particles were collected 48 h after the medium change and filtered through a 0.45-μm PVDF syringe filter. The filtered supernatant was used directly to infect cells. Aliquots were snap-frozen and stored at −80° C. For transduction, lentiviral particles were diluted in complete growth medium supplemented with 10 μg/ml polybrene (Sigma) and added to cells.

### Chemical Inhibitors

AM1882 (KIF18Ai; Amgen) was dissolved in DMSO and used at a final concentration of 250 nM or 500 nM, unless otherwise indicated. Reversine (MPS1i; Axon Med Chem) was dissolved in DMSO and used at a final concentration of 500 nM, unless otherwise indicated. RO-3306 (Sigma) was dissolved in DMSO and used at a final concentration of 10 μM, unless otherwise indicated. Taxol (Sigma) was dissolved in DMSO and used at a final concentration of 10 μM. MG132 (Sigma) was dissolved in DMSO and used at a final concentration of 10 μM. Nocodazole (Sigma) was dissolved in DMSO and used at a final concentration of 3.3 μM. Dimethylenastron (DMN; Sigma) was dissolved in DMSO and used at a final concentration of 2 μM.

### Antibody Techniques

For immunoblot analyses, protein samples were separated by SDS-PAGE, transferred onto nitrocellulose membranes with a Trans-Blot Turbo Transfer System (BioRad) and then probed with the following primary antibodies: α-Tubulin (rat; Thermo Fischer, YL1/2, #MA1-80017; 1:1000-1:3000), APC4 (rabbit, Bethyl Laboratories, #A301-176A; 1:1000), Cyclin B1 (mouse; SantaCruz, GNS1, #SC245; 1:750-1:1000), HA (rat; Roche, #ROAHAHA; 1:1000), HSET (rabbit; Cell Signaling, #12313; 1:1000), KIF18A (rabbit; Bethyl, #A301-080; 1:1000), MAD1 (mouse; Millipore Sigma, #MABE867; 1:1000), TRIP13 (mouse; SantaCruz, A-7, #SC514285; 1:1000), UBE2S (rabbit; Proteintech, #14115-1AP; 1:1000), Vinculin (mouse; Santa Cruz, 7F9, #SC73614; 1:1000). Proteins were then detected using HRP-conjugated anti-mouse (horse; Cell Signaling, #7076; 1:1000), HRP-conjugated anti-rat (goat; Cell Signaling, #7077; 1:1000), or HRP-conjugated anti-rabbit (goat; Cell Signaling, #7074; 1:1000) and SuperSignal West chemiluminescence substrate (Pico PLUS/Femto, ThermoFischer). Signals were visualized and acquired using a Genesys G: Box Chemi-XX6 system (Syngene).

For immunofluorescence, cells were grown on 12-mm glass coverslips. If mitotic cells were being analyzed, they were washed for 30 s in microtubule stabilizing buffer (MTSB; 1 mM EGTA, 1 mM MgSO4, 100 mM PIPES, 30% glycerol) before fixation in 4% formaldehyde at room temperature for 10 min. Otherwise cells were directly fixed in MeOH at −20° C. for 10 min. Cells were blocked in 2.5% FBS, 200 mM glycine, and 0.1% Triton X-100 in PBS for 1 hr. Antibody incubations were conducted in the blocking solution for 1 hr. DNA was stained with DAPI and cells were mounted in ProLong Gold Antifade (Invitrogen). Staining was performed with the following primary antibodies: α-Tubulin (rat; Thermo Fischer, YL1/2, #MA1-80017; 1:1000), BUBR1 (sheep; kind gift of S. Taylor (Univ, of Manchester) #SBR1.1; 1:1000), CENP-A (mouse; GeneTex, #GTX13939; 1:2000), CEP192-Cy5 (directly labelled goat; raised against CEP192 amino acids 1-211, this study; 1:1,000), KIF18A (rabbit; Bethyl, #A301-080; 1:1000), MAD1 (mouse; Millipore Sigma, #MABE867; 1:1000), Phospho-Histone H2A.X (Ser139) (rabbit; Cell Signaling, #2577; 1:800). Immunofluorescence images were collected using a Deltavision Elite system (GE Healthcare) controlling a Scientific CMOS camera (pco.edge 5.5). Acquisition parameters were controlled by SoftWoRx suite (GE Healthcare). Images were collected at room temperature (25° C.) using an Olympus 40×1.4 NA oil objective or an Olympus 100×1.4 NA oil objective at 0.2-μm z-sections. Images were acquired using Applied Precision immersion oil (N=1.516).

Quantification of location and signal intensity at individual kinetochores was achieved using a custom FIJI macro on deconvolved 3D image z-stacks. Briefly, individual kinetochore regions were defined using tools from the 3D ImageJ Suite plugin 63 on manually-thresholded BUBR1 image stacks. 3D segmented kinetochore regions were then used to measure BUBR1 and MAD1 intensity. Background signal was determined as the median signal of the non-kinetochore area throughout the entire z-stack and subtracted from BUBR1 and MAD1 measurements respectively. The location of spindle poles was identified using a similar methodology using the CEP192 channel. Downstream processing and quality control of kinetochore intensity data was managed in R. Non- or missegmented-kinetochores were excluded based on size to remove instances where multiple kinetochores segmented together (>1.5× interquartile range (IQR) of average volume) or by coincidence of CEP192 signal (spindle poles). Cells with aberrant kinetochore numbers (outside 1.5× IQR of average kinetochore number) were also excluded from the analysis. MAD1+kinetochores were determined as being above the 90th, 95th, or 99th percentile of MAD1 signal in the DMSO condition as indicated.

### Live Cell Microscopy

Fluorescent cell lines were seeded into either 4-chamber, 35-mm glass-bottom culture dishes (Greiner), 4-well chamber slides (Ibidi), or 8-well chamber slides (Ibidi). The day of the experiment, cells were transferred to CO2-independent base medium (ThermoFischer) with the appropriate additives for each cell line and maintained at 37° C. in an environmental control station. Long-term time-lapse imaging was performed using a Deltavision Elite system (GE Healthcare) controlling a Scientific CMOS camera (pco.edge 5.5.). Images were acquired with an Olympus 40×1.4 NA oil objective. For general mitotic phenotypes, cells were treated as indicated, then images were captured every 5 min in 9×3 μm z-sections in respective fluorescent channels and by differential inference contrast. For CyclinB1-EYFP experiments, cells were treated as indicated, then images were captured every 2.5 min in 9×3 μm z-sections in respective fluorescent channels and by differential inference contrast. Phenotypic evaluation and intensity measurements were taken from maximum intensity projected 2D time-lapse images. Mitotic duration was calculated as the time taken from nuclear envelope breakdown to the onset of anaphase. Metaphase to anaphase duration was calculated as the time from the appearance of the last, uninterrupted metaphase plate to the onset of anaphase. Analysis of Cyclin B1-EYFP mitotic intensity was measured from NEBD through 30 min past anaphase using FIJI. Intensity traces were manually aligned to the metaphase to anaphase inflection point, then normalized in R. If traces never inflected, they were aligned to 30 min past NEBD.

For high-resolution live-cell imaging of mitosis, cells were seeded into 4-chamber, 35-mm glass-bottom culture dishes (Greiner) and maintained at 37° C. and 5% CO2 in an environmental control station. Imaging was performed using a Lecia SP-8 confocal microscope, equipped with a resonance scanner, and 405-nm, 488-nm, 552-nm and 638-nm laser lines. Images were acquired with a Leica 40×1.3 NA oil objective. For general mitotic phenotypes, cells were treated as indicated, then images were captured every 5 min in 8×3 μm z-sections in respective fluorescent channels and by differential inference contrast. For HeLa EGFP-BUBR1 movies, cells were simultaneously treated with the indicated drugs and MG132 to prevent anaphase onset in all conditions, then images were captured every 2.5 min in 35×0.75 μm z-sections for 2 hr. All movies were deconvolved using the LIGHTNING adaptive approach and collapsed into 2D using a maximum intensity projection. BUBR1 foci number and intensity were determined using FIJI.

For live cell imaging of PA-GFP-α-Tubulin, cells were seeded into 4-chamber, 35-mm glass-bottom culture dishes (Greiner) and maintained at 37° C. and 5% CO2 in an environmental control station. Imaging was performed using a Lecia SP-8 confocal microscope, equipped with a resonance scanner, and 405 nm, 488 nm, 552 nm and 638 nm laser lines. Images were acquired with a 63×1.4 NA oil objective. Metaphase cells were identified using H2B-iRFP signal or differential inference contrast. The region immediately distal to the metaphase plate was marked for photo-activation. One pre-photoactivation frame (4×1 μm z-sections) was collected, immediately followed by PA-GFP-α-Tubulin photoactivation using 5 pulses of the 405 nm laser at 50% intensity. Frames were then collected as before every 10 s for 6 min. Cells were excluded from analysis if they entered anaphase during the time-lapse. Spindle pole position was manually annotated in maximum intensity projected 2D time-lapse images and measurements for GFP intensity were taken along the spindle pole axis in FIJI. Downstream analysis of tubulin dynamics was managed in R. Before all analyses, the non-photoactivated side of the spindle was used as background subtraction for the activated side. For flux measurements, the location of the maximum GFP intensity band along the spindle pole axis was determined in an automated fashion from smoothed intensity data.

### Cell Growth and Viability Techniques

To measure 5-day endpoint cell growth and viability, cells were plated in triplicate for each condition in 200 μL media in a 96-well plate (RPE1 at 600 cells/well; DLD1, HCT116, HCC1806, HeLa, MCF10A, and OVCAR-8 at 1200 cells/well; MCF7 at 5000 cells/well; OVCAR-3 at 8500 cells/well; and MDA-MB-157 at 15000 cells/well). The following day, drugs were added as indicated and cells were cultured for 5 days. To assay viability, 10 μl of 5 mg/ml Thiazolyl blue tetrazolium bromide (MTT; Abcam) solution in PBS was added to each well and incubated for 4.5 hr. 100 μL 10% SDS, 0.01M HCl solution was then added to each well to dissolve the formazan crystals. Absorbance was measured with a spectrophotometer at 570 nm. % Viability=(Signal[sample]−Signal[DMN])/(Signal [DMSO]−Signal [DMN])×100 unless specifically noted. % Toxicity=100%−Viability. % Viability rescue=(Signal [sample, KIF18Ai]−Signal[WT, KIF18Ai])/(Signal[WT, DMSO]−Signal [WT, KIF18Ai])×100. Drug titration curves were derived using the equation for “[Inhibitor] vs. response−Variable slope (four-parameter)” in GraphPad Prism 7 for Mac OS X (GraphPad Software, La Jolla, USA).

For longitudinal live cell confluence and growth measurements, cells were plated in triplicate for each condition in 200 μL media in a 96-well plate (RPE1 at 600 cells/well; DLD1, HCT116, HCC1806, HeLa, MCF10A, and OVCAR-8 at 1200 cells/well; MCF7 at 5000 cells/well; OVCAR-3 at 8500 cells/well; and MDA-MB-157 at 15000 cells/well). The following day, drugs were added, and confluency measurements were taken using the CellCyte X live cell incubator imaging system (Echo) every 4 hours for 7 days.

### Flow Cytometry

DNA ploidy analysis was assessed using propidium iodide (PI) staining. Cells were trypsinized and washed with 1% BSA in PBS (1,500 rpm, 5 min) before being fixed in 70% ethanol. Following 3 washes with 1% BSA in PBS, a 20 min incubation at 37° C. in a solution of 10 μg/mL PI and 0.1 mg/mL RNaseA in PBS was performed. Samples were analyzed using a FACSCalibur Flow Cytometer (BD Biosciences) and data processing was done using FlowJo software.

scDNA Sequencing

Single cell DNA sequencing was performed on RPE1, HCT116, and MCF10A cells grown for 7 days in DMSO or KIF18Ai for 7 days. Sequencing was performed on whole cells including micronuclei as described previously⁶⁴, and analyzed with the AneuFinder algorithm⁶⁵.

### CRISPR-Cas9 Genome-Wide Screens

Pooled genome-wide CRISPR-Cas9 knockout screens in HCC-1806 and OVCAR-3 cells were performed as described previously 66-68. HCC-1806 cells were infected with lentiCas9-Blast (Addgene; #52962). OVCAR-3 were infected with a lentiviral construct containing Cas9 and a Hygromycin resistance cassette. Positive selection of transduced cells was performed 2 days post-transfection with 400 μg/mL Hygromycin or 5 μg/mL blasticidin, respectively. Monoclonal cell lines were isolated by limiting dilution and Cas9 expression was validated by immunoblotting.

The human Brunello CRISPR knockout sgRNA library was purchased from Addgene (a gift of David Root and John Doench; #73178) and plasmid DNA amplified according to the manufacturer's instructions. To produce virus, the Brunello pooled plasmid library and the lentiviral packaging plasmids psPAX2 and pMD2.G were co-transfected into 40×15 cm culture dishes of HEK293FT cells. 6×106 HEK293FT cells were seeded into a poly-l-Lysine-coated 15 cm culture dish the day before transfection. For each 15 cm dish, the following DNA was diluted in 1.2 mL OptiMEM (Thermo Fisher Scientific): 9 μg lentiviral vector, 12 μg psPAX2, and 3 μg pMD2.G. Separately, 70 μl of 1 μg/μL 25-kD polyethylenimine (PEI) (Sigma-Aldrich) was diluted into 1.2 mL OptiMEM and incubated at room temperature for 5 min. After incubation, the DNA and PEI mixtures were combined and incubated at room temperature for 20 min. During this incubation, the culture media was replaced with 16 mL pre-warmed DMEM+1% FBS. The transfection mixture was then added dropwise to the 15 cm dish. Viral particles were harvested at 24, 48, and 72 hr after the media change. Media collected from 24, 48, and 72 hr were pooled and filtered through a 0.45 μm PVDF syringe filter. The media was then concentrated using Amicon Ultra-15 Centrifugal Filter Unit with Ultracel-50 membrane (EMD Millipore Corporation cat #UFC905024). The virus was then frozen and stored at −80° C.

Cells were transduced with the Brunello library via spinfection as previously described 68. To determine the optimal virus volumes for achieving an MOI ˜0.3, each new batch of virus was titered by spinfecting 3×106 cells with several different volumes of virus. Briefly, 3×106 cells per well were seeded into a 12 well plate in growth media supplemented with 10 μg/mL polybrene. Each well received a different titrated virus amount (between 5 and 50 μL) along with a no-transduction control. The plate was centrifuged at 1000 g for 2 hr at 35° C. After the spin, media was aspirated, and fresh growth media was added. The following day, cells were counted, and each well was split into duplicate wells. One well received 3 μg/mL puromycin (Sigma) for 3 days. Cells were counted and the percent transduction was calculated as the cell count from the replicate with puromycin divided by the cell count from the replicate without puromycin multiplied by 100. The virus volume yielding a MOI closest to 0.3 was chosen for large-scale transductions.

For the pooled screens, a theoretical library coverage of ≥250 cells per sgRNA was maintained at every step. Cells were infected at MOI ˜0.3 and selected with puromycin at 3 μg/ml for 3 days. MOI was calculated using a control well infected in parallel following the procedure outlined above. Infected cells were expanded under puromycin selection for 6 or 7 days and subsequently seeded into 15 cm dishes a day prior to treatment. KIF18Ai doses that corresponded to individual LD80 for HCC-1806 cells (25 nM) or OVCAR-3 (20 nM) cells were used while DMSO vehicle served as the negative control. Cell pallets were taken after 21 days for HCC-1806 cells, 40 days for OVCAR-3 cells, and 4 days for OVCAR-8 cells, at which point the screen was terminated.

Genomic DNA was isolated using the QIAamp Blood Maxi Kit (Qiagen) per manufacturer's instructions. Genome-integrated sgRNA sequences for each sample were amplified and prepared for Illumina sequencing using a two-step PCR procedure as previously described⁶⁸. For the first PCR, a region containing the sgRNA cassette was amplified using primers specific to the sgRNA-expression vector (lentiGuide-PCR1-F: 5′-aatggactatcatatgcttaccgtaacttgaaagtatttcg-3′ (SEQ ID NO: 58); lentiGuide-PCR1-R: 5′-ctttagtttgtatgtctgttgctattatgtctactattctttcc-3′ (SEQ ID NO: 59)). The thermocycling parameters for the first PCR were as follows: 98° C. for 1 min, 20 cycles of (98° C. for 30 s, 65° C. for 30 s, 72° C. for 30 s), and 72° C. for 1 min. The resulting amplicons for each sample were pooled and purified using AMPURE XP beads (Beckman Coulter) with a bead to sample ratio of 0.6× and 1.0× for double size selection to exclude primers and genomic DNA. Primers for the second PCR include Illumina adapter sequences, a variable length sequence to increase library complexity and a 8 bp barcodes for multiplexing of different biological samples (

F2: 5′-aatgatacggcgaccaccgagatctacactctttccctacacgacgctcttccgatct ((SEQ ID NO: 60)-[4-7 bp random nucleotides]-[8 bp barcode]-tcttgtggaaaggacgaaacaccg-3′ ((SEQ ID NO: 61); R2: 5′-caagcagaagacggcatacgagatgtgactggagttcagacgtgtgctcttccgatcttctactattctttcccctgcactgt-3′ (SEQ ID NO: 62)). 2.5 μL of the product from the first PCR reaction was used, and the thermocycling parameters for the second PCR were as follows: 98° C. for 30 s, 12 cycles of (98° C. for 1 s, 70° C. for 5 s, 72° C. for 35 s). Second PCR products were pooled, purified using AMPURE XP beads with a bead to sample ratio of 1.8× and quantified using the Qubit dsDNA BR Assay Kit (Thermo Fischer Scientific). Diluted libraries with 5% PhiX were sequenced with MiSeq (Illumina).

Sequencing data were processed for sgRNA representation using custom scripts. Briefly, sequencing reads were first demultiplexed using the barcodes in the forward primer and then trimmed to leave only the 20 bp sgRNA sequences. The spacer sequences were then mapped to the spacers of the designed sgRNA library using Bowtie 69. For mapping, a maximum of one mismatch was allowed in the 20 bp sgRNA sequence. Mapped sgRNA sequences were then quantified by counting the total number of reads. The total numbers of reads for all sgRNAs in each sample were normalized.

We used the MaGeCK scoring algorithm (model-based analysis of genome-wide CRISPR-Cas9 knockout) to analyze and the rank the genes from the screens 70. β-scores and FDR values were also derived for each gene between DMSO or KIF18Ai conditions. Genes with a β-score outside 1.5 standard deviations from the population mean and with an FDR cutoff of ≤0.3 were taken forward for further validation. Graphing and downstream analysis was performed in R.

CRISPR-Cas9 pooled, knockout screens in OVCAR-8 cells were performed by Cellecta, Inc. In brief, 1×108 OVCAR-8 Cas9 cells were infected with a proprietary lentiviral sgRNA library at MOI 0.4-0.5 and cultured for 4 days. Transduced cells were then selected with puromycin for 3 days. Two days later, samples were treated with either 11 nM KIF18Ai (IC50), 33 nM KIF18Ai (IC90) or DMSO. Cells were then cultured for 4 days before DNA isolation. sgRNA abundance in each population was determined using deep sequencing. β-scores and FDR values between DMSO and KIF18Ai conditions were used for downstream analysis. Genes with a β-score outside 1.5 standard deviations from the population mean and with an FDR cutoff of ≤0.1 were taken forward for further validation. Graphing and downstream analysis was performed in R

### REFERENCES

The following is a list of references cited in Examples 8 and 9.
1 Checchi, P. M., et al., Trends in Pharmacological Sciences 24, 361-365, doi: 10.1016/s0165-6147 (03) 00161-5 (2003).
2 Jordan et al., Nature Reviews Cancer 4, 253-265, doi: 10.1038/nrc1317 (2004).
3 Marupudi, N. I. et al. Expert Opinion on Drug Safety 6, 609-621, doi: 10.1517/14740338.6.5.609 (2007).
4 Shemesh, O. A. & Spira, M. E., Acta Neuropathologica 119, 235-248, doi: 10.1007/s00401-009-0586-0 (2009).
5 Gornstein, E. & Schwarz, T. L., Neuropharmacology 76, 175-183, doi: 10.1016/j.neuropharm.2013.08.016 (2014).
6 Klein, I. & Lehmann, H., Toxics 9, doi: 10.3390/toxics9100229 (2021).
7 Čermák, V. et al. European Journal of Cell Biology 99, doi: 10.1016/j.ejcb.2020.151075 (2020).
8 Chan, K. S., et al., Cell Death & Disease 3, e411-e411, doi: 10.1038/cddis.2012.148 (2012).
9 Serrano-del Valle, A. et al., Biochemical Pharmacology 190, doi: 10.1016/j.bcp.2021.114655 (2021).
10 Quinton, R. J. et al., Nature 590, 492-497, doi: 10.1038/s41586-020-03133-3 (2021).
11 Marquis, C. et al., Nature Communications 12, doi: 10.1038/s41467-021-21447-2 (2021).
12 Cohen-Sharir, Y. et al., Nature 590, 486-491, doi: 10.1038/s41586-020-03114-6 (2021).
13 Mayr, M. I. et al., Current Biology 17, 488-498, doi: 10.1016/j.cub.2007.02.036 (2007).
14 Stumpff, J., et al., Developmental Cell 14, 252-262, doi: 10.1016/j.devcel.2007.11.014 (2008).
15 Zhu, C. et al., Molecular Biology of the Cell 16, 3187-3199, doi: 10.1091/mbc.e05-02-0167 (2005).
16 Du, Y., et al., Current Biology 20, 374-380, doi: 10.1016/j.cub.2009.12.049 (2010).
17 Lin, Y., et al., Chromosoma 129, 99-110, doi: 10.1007/s00412-020-00736-7 (2020).
18 Janssen, L. M. E. et al., Current Biology 28, 2685-2696.e2684, doi: 10.1016/j.cub.2018.06.026 (2018).
19 Fonseca, C. L. et al., Journal of Cell Biology 218, 1148-1163, doi: 10.1083/jcb.201807228 (2019).
20 Sepaniac, L. A. et al., Journal of Cell Biology 220, doi: 10.1083/jcb.202101165 (2021).
21 Payton, M. et al., Small-Molecule Inhibition of Kinesin KIF18A Reveals a Mitotic Vulnerability Enriched in Chromosomally Unstable Cancers. in review (2023).
22 Prasad, K. et al., Cancer Research 82, 1736-1752, doi: 10.1158/0008-5472.Can-21-2065 (2022).
23 Tamayo, N. A. et al., Journal of Medicinal Chemistry 65, 4972-4990, doi: 10.1021/acs.jmedchem.1c02030 (2022).
24 Locke, J. et al., Proceedings of the National Academy of Sciences 114, doi: 10.1073/pnas.1712169114 (2017).
25 Waterhouse, A. M., Procter, J. B., Martin, D. M. A., Clamp, M. & Barton, G. J. Jalview Version 2-a multiple sequence alignment editor and analysis workbench. Bioinformatics 25, 1189-1191, doi: 10.1093/bioinformatics/btp033 (2009).
26 Murray, A. W. Recycling the Cell Cycle. Cell 116, 221-234, doi: 10.1016/s0092-8674 (03) 01080-8 (2004).
27 Brito, D. A. & Rieder, C. L. Mitotic Checkpoint Slippage in Humans Occurs via Cyclin B Destruction in the Presence of an Active Checkpoint. Current Biology 16, 1194-1200, doi: 10.1016/j.cub.2006.04.043 (2006).
28 Müller, C. et al. Inhibitors of kinesin Eg5: antiproliferative activity of monastrol analogues against human glioblastoma cells. Cancer Chemotherapy and Pharmacology 59, 157-164, doi: 10.1007/s00280-006-0254-1 (2006).
29 Collin, P., Nashchekina, O., Walker, R. & Pines, J. The spindle assembly checkpoint works like a rheostat rather than a toggle switch. Nature Cell Biology 15, 1378-1385, doi: 10.1038/ncb2855 (2013).
30 Chen, R.-H., Shevchenko, A., Mann, M. & Murray, A. W. Spindle Checkpoint Protein Xmad1 Recruits Xmad2 to Unattached Kinetochores. Journal of Cell Biology 143, 283-295, doi: 10.1083/jcb.143.2.283 (1998).
31 Taylor, S. S., Hussein, D., Wang, Y., Elderkin, S. & Morrow, C. J. Kinetochore localisation and phosphorylation of the mitotic checkpoint components Bub1 and BubR1 are differentially regulated by spindle events in human cells. Journal of Cell Science 114, 4385-4395, doi: 10.1242/jcs.114.24.4385 (2001).
32 Vigneron, S. et al. Kinetochore Localization of Spindle Checkpoint Proteins: Who Controls Whom? Molecular Biology of the Cell 15, 4584-4596, doi: 10.1091/mbc.e04-01-0051 (2004).
33 Nijenhuis, W., Vallardi, G., Teixeira, A., Kops, G. J. P. L. & Saurin, Adrian T. Negative feedback at kinetochores underlies a responsive spindle checkpoint signal. Nature Cell Biology 16, 1257-1264, doi: 10.1038/ncb3065 (2014).
34 Moura, M. et al. Protein Phosphatase 1 inactivates Mps1 to ensure efficient Spindle Assembly Checkpoint silencing. eLife 6, doi: 10.7554/eLife.25366 (2017).
35 De Wever, V. et al. The human mitotic kinesin KIF18A binds protein phosphatase 1 (PP1) through a highly conserved docking motif. Biochemical and Biophysical Research Communications 453, 432-437, doi: 10.1016/j.bbrc.2014.09.105 (2014).
36 Häfner, J., Mayr, M. I., Möckel, M. M. & Mayer, T. U. Pre-anaphase chromosome oscillations are regulated by the antagonistic activities of Cdk1 and PP1 on Kif18A. Nature Communications 5, doi: 10.1038/ncomms5397 (2014).
37 Hayward, D., Alfonso-Perez, T. & Gruneberg, U. Orchestration of the spindle assembly checkpoint by CDK1-cyclin B1. FEBS Letters 593, 2889-2907, doi: 10.1002/1873-3468.13591 (2019).
38 Yu, H. & Yao, X. Cyclin B1: conductor of mitotic symphony orchestra. Cell Research 18, 218-220, doi: 10.1038/cr.2008.20 (2008).
39 Allan, L. A. et al. Cyclin B1 scaffolds MAD1 at the kinetochore corona to activate the mitotic checkpoint. The EMBO Journal 39, doi: 10.15252/embj.2019103180 (2020).
40 Kops, G. J. P. L. & Gassmann, R. Crowning the Kinetochore: The Fibrous Corona in Chromosome Segregation. Trends in Cell Biology 30, 653-667, doi: 10.1016/j.tcb.2020.04.006 (2020).
41 Allan, L. A. et al. Cyclin B1 scaffolds MAD1 at the kinetochore corona to activate the mitotic checkpoint. Embo J 39, e103180, doi: 10.15252/embj.2019103180 (2020).
42 Raisch, T. et al. Structure of the RZZ complex and molecular basis of Spindly-driven corona assembly at human kinetochores. The EMBO Journal 41, doi: 10.15252/embj.2021110411 (2022).
43 Steblyanko, Y. et al. Microtubule poleward flux in human cells is driven by the coordinated action of four kinesins. The EMBO Journal 39, doi: 10.15252/embj.2020105432 (2020).
44 Cai, S., Weaver, L. N., Ems-McClung, S. C., Walczak, C. E. & Bloom, K. S. Kinesin-14 Family Proteins HSET/XCTK2 Control Spindle Length by Cross-Linking and Sliding Microtubules. Molecular Biology of the Cell 20, 1348-1359, doi: 10.1091/mbc.e08-09-0971 (2009).
45 Jia, L., Kim, S. & Yu, H. Tracking spindle checkpoint signals from kinetochores to APC/C.
Trends in Biochemical Sciences 38, 302-311, doi: 10.1016/j.tibs.2013.03.004 (2013).
46 Barisic, M., Rajendraprasad, G. & Steblyanko, Y. The metaphase spindle at steady state-Mechanism and functions of microtubule poleward flux. Seminars in Cell & Developmental Biology 117, 99-117, doi: 10.1016/j.semcdb.2021.05.016 (2021).
47 Samora, C. P. & McAinsh, A. D. in Microtubule Dynamics Methods in Molecular Biology Ch.
Chapter 16, 223-233 (2011).
48 Zhai, Y., Kronebusch, P. J. & Borisy, G. G. Kinetochore microtubule dynamics and the metaphase-anaphase transition. Journal of Cell Biology 131, 721-734, doi: 10.1083/jcb.131.3.721 (1995).
49 Buster, D. W., Zhang, D., Sharp, D. J. & Zheng, Y. Poleward Tubulin Flux in Spindles: Regulation and Function in Mitotic Cells. Molecular Biology of the Cell 18, 3094-3104, doi: 10.1091/mbc.e06-11-0994 (2007).
50 Bakhoum, S. F., Genovese, G. & Compton, D. A. Deviant Kinetochore Microtubule Dynamics Underlie Chromosomal Instability. Current Biology 19, 1937-1942, doi: 10.1016/j.cub.2009.09.055 (2009).
51 Sivakumar, S. & Gorbsky, G. J. Spatiotemporal regulation of the anaphase-promoting complex in mitosis. Nature Reviews Molecular Cell Biology 16, 82-94, doi: 10.1038/nrm3934 (2015).
52 Mocciaro, A. & Rape, M. Emerging regulatory mechanisms in ubiquitin-dependent cell cycle control. Journal of Cell Science 125, 255-263, doi: 10.1242/jcs.091199 (2012).
53 Lischetti, T. & Nilsson, J. Regulation of mitotic progression by the spindle assembly checkpoint. Molecular & Cellular Oncology 2, doi: 10.4161/23723548.2014.970484 (2015).
54 Rieder, C. L., Cole, R. W., Khodjakov, A. & Sluder, G. The checkpoint delaying anaphase in response to chromosome monoorientation is mediated by an inhibitory signal produced by unattached kinetochores. Journal of Cell Biology 130, 941-948, doi: 10.1083/jcb.130.4.941 (1995).
55 Tsherniak, A. et al. Defining a Cancer Dependency Map. Cell 170, 564-576.e516, doi: 10.1016/j.cell.2017.06.010 (2017).
56 Meyers, R. M. et al. Computational correction of copy number effect improves specificity of CRISPR-Cas9 essentiality screens in cancer cells. Nature Genetics 49, 1779-1784, doi: 10.1038/ng.3984 (2017).
57 Krill-Burger, J. M. et al. Partial gene suppression improves identification of cancer vulnerabilities when CRISPR-Cas9 knockout is pan-lethal. bioRxiv, doi: 10.1101/2022.03.02.482624 (2022).
58 Garnett, M. J. et al. UBE2S elongates ubiquitin chains on APC/C substrates to promote mitotic exit. Nature Cell Biology 11, 1363-1369, doi: 10.1038/ncb1983 (2009).
59 Craney, A. et al. Control of APC/C-dependent ubiquitin chain elongation by reversible phosphorylation. Proceedings of the National Academy of Sciences 113, 1540-1545, doi: 10.1073/pnas.1522423113 (2016).
60 Dempster, J. M. et al. Gene expression has more power for predicting in vitro cancer cell vulnerabilities than genomics. bioRxiv, doi: 10.1101/2020.02.21.959627 (2020).
61 Sansregret, L. et al. APC/C Dysfunction Limits Excessive Cancer Chromosomal Instability. Cancer Discovery 7, 218-233, doi: 10.1158/2159-8290.Cd-16-0645 (2017).
62 Thu, K. L. et al. Disruption of the anaphase-promoting complex confers resistance to TTK inhibitors in triple-negative breast cancer. Proceedings of the National Academy of Sciences 115, doi: 10.1073/pnas.1719577115 (2018).
63 Ollion, J., Cochennec, J., Loll, F., Escudé, C. & Boudier, T. TANGO: a generic tool for high-throughput 3D image analysis for studying nuclear organization. Bioinformatics 29, 1840-1841, doi: 10.1093/bioinformatics/btt276 (2013).
64 van den Bos, H. et al. in Cellular Senescence Methods in Molecular Biology Ch. Chapter 15, 159-190 (2019).
65 Bakker, B. et al. Single-cell sequencing reveals karyotype heterogeneity in murine and human malignancies. Genome Biology 17, doi: 10.1186/s13059-016-0971-7 (2016).
66 Chen, S. et al. Genome-wide CRISPR Screen in a Mouse Model of Tumor Growth and Metastasis. Cell 160, 1246-1260, doi: 10.1016/j.cell.2015.02.038 (2015).
67 Shalem, O. et al. Genome-Scale CRISPR-Cas9 Knockout Screening in Human Cells. Science 343, 84-87, doi: 10.1126/science.1247005 (2014).
68 Lambrus, B. G. et al. A USP28-53BP1-p53-p21 signaling axis arrests growth after centrosome loss or prolonged mitosis. Journal of Cell Biology 214, 143-153, doi: 10.1083/jcb.201604054 (2016).
69 Langmead, B., Trapnell, C., Pop, M. & Salzberg, S. L. Ultrafast and memory-efficient alignment of short DNA sequences to the human genome. Genome Biology 10, doi: 10.1186/gb-2009 Oct. 3-r25 (2009).
70 Li, W. et al. MAGeCK enables robust identification of essential genes from genome-scale CRISPR/Cas9 knockout screens. Genome Biology 15, doi: 10.1186/s13059-014-0554-4 (2014).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method of determining a treatment for a subject having a neoplastic disease or identifying a subject having a neoplastic disease as sensitive to treatment with a KIF18A inhibitor, said method comprising assaying a sample obtained from the subject for
a. Spindle Assembly Checkpoint (SAC) activity,
b. ploidy,
c. whole genome doubling (WGD),
d. Anaphase Promoting Complex (APC/C) activity, or
e. a combination thereof
wherein the treatment determined for the subject comprises, consists essentially of, or consists of a KIF18A inhibitor, when the sample is positive for (a) increased SAC activity, (b) high ploidy, (c) WGD, (d) low APC/C activity, or (e) a combination thereof.

2. A method of treating a subject having a neoplastic disease, said method comprising
a. assaying a sample obtained from the subject for
i. SAC activity,
ii. ploidy,
iii. WGD,
iv. APC/C activity, or
v. a combination thereof, and
b. administering a KIF18A inhibitor to the subject when the sample is positive for (a) increased SAC activity, (b) high ploidy, (c) WGD, (d) low APC/C activity, or (e) a combination thereof as assayed in (a),
optionally, wherein the method further comprises obtaining the sample from the subject.

3. A method of treating a subject having a neoplastic disease, wherein the subject comprises cells that are positive for (a) increased SAC activity, (b) high ploidy, (c) WGD, (d) low APC/C activity, or (e) a combination thereof, said method comprising administering a KIF18A inhibitor to the subject.

4. (canceled)

5. A method of treating a subject with a cancer comprising one or more whole genome duplication or whole genome doubling (WGD) events, said method comprising:
a. assaying APC/C activity in a tumor cell obtained from the subject or lowering APC/C activity in the subject, optionally, by inhibiting expression of UBE2S;
b. administering to the subject a KIF18A inhibitor when the APC/C activity measured in (a) is low, and optionally further administering to the subject an agent that lowers APC/C activity in the subject.

6-7. (canceled)

8. The method of claim 2, wherein the assaying step comprises assaying the sample for expression levels of RNA or protein encoded by one or more of the following genes: ANAPC1, ANAPC2, ANAPC4, ANAPC5, ANAPC7, ANAPC10, ANAPC11, ANAPC13, ANAPC15, ANAPC16, CDC16, CDC23, CDC26, CDC27, UBE2C, UBE2D1, and UBE2S.

9. The method of claim 2, wherein the assaying step comprises assaying the sample for assaying expression levels of RNA or protein encoded by one or more of the following genes: BUB1, BUB1B, BUB3, AURKB, CCNB1, MAD1L1, MAD2L1, MAD2L1GP, PPP1CA, PPP1CB, PPP1CC, TRIP13, TPR, USP44, ZNF207, ZW10, and ZWILCH.

10. The method of claim 2, wherein the assaying step comprises measuring ploidy and/or WGD via chromosome counting (via e.g., karyotyping, parallel sequencing, comparative genomic hybridization (CGH), microarrays) high throughput sequencing (HTS), or flow cytometry.

11. The method of claim 2, wherein the sample comprises cancer cells, tumor cells, non-tumor cells, blood, blood cells, or plasma, optionally, wherein the sample comprises germline cancer cells or somatic cancer cells.

12. The method of claim 2, wherein the neoplastic disease is a cancer, optionally, breast cancer, ovarian cancer, endometrial cancer, lung cancer, or prostate cancer.

13. The method of claim 12, wherein the neoplastic disease is triple-negative breast cancer (TNBC), non-luminal breast cancer, high-grade serous ovarian cancer (HGSOC), endometrial cancer, optionally, serous endometrial cancer, or non-small-cell lung cancer.

14. The method of claim 2, wherein the sample is positive for one or more whole genome duplication or whole genome doubling (WGD) events.

15. The method of claim 2, wherein treatment with or administration of the KIF18A inhibitor induces at least 50% tumor regression, compared to a control.

16. The method of claim 2, wherein treatment with or administration of the KIF18A inhibitor induces at least 75% tumor regression, compared to a control.

17. The method of claim 2, wherein treatment with or administration of the KIF18A inhibitor induces at least 80% or 85% tumor regression, compared to a control.

18. The method of claim 2, wherein treatment with or administration of the KIF18A inhibitor induces at least 90% or 95% tumor regression, compared to a control.

19. The method of claim 2, wherein the KIF18A inhibitor is Compound C9, which is 4-(N-(tert-butyl) sulfamoyl)-N-(3-(N-(tert-butyl) sulfamoyl)phenyl)-2-(6-azaspiro[2.5]octan-6-yl)benzamide and/or has the following structure:

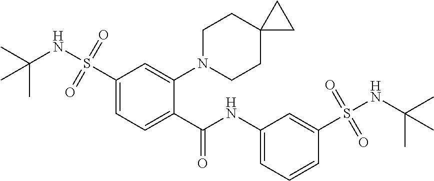 [SMILES: CC(C)(C)NS(=O)(=O)c1cccc(NC(=O)c2ccc(S(=O)(=O)NC(C)(C)C)cc2N2CCC3(CC2)CC3)c1]

20. The method of claim 2, wherein the KIF18A inhibitor is N-(2-(4,4-Difluoropiperidin-1-yl)-6-methylpyrimidin-4-yl)-4-((2-hydroxyethyl) sulfonamido)-2-(6-azaspiro[2.5]octan-6-yl)benzamide and/or has the following structure:

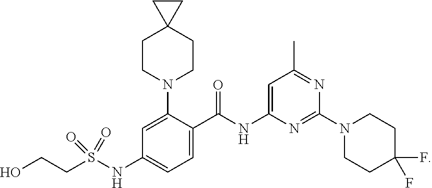 [SMILES: Cc1cc(NC(=O)c2ccc(NS(=O)(=O)CCO)cc2N2CCC3(CC2)CC3)nc(N2CCC(F)(F)CC2)n1]

21. The method of claim 2, wherein the KIF18A inhibitor is administered for oral administration, optionally once a day.
